(19) United States
(12) Patent Application Publication
(10) Pub. No.: US 2013/0005806 A1
(43) Pub. Date: Jan. 3, 2013

(54) ANALYSIS OF TMLHE AND CARNITINE BIOSYNTHESIS FOR AUTISM DIAGNOSIS

(76) Inventors: Arthur L. Beaudet, Houston, TX (US); Frederic M. Vaz, Amsterdam (NL)

(21) Appl. No.: 13/525,677

(22) Filed: Jun. 18, 2012

Related U.S. Application Data
(60) Provisional application No. 61/498,067, filed on Jun. 17, 2011.

(51) Int. Cl. A61K 31/205; A61P 25/00; A61K 31/221

(52) U.S. Cl. 514/546; 514/556

(57) ABSTRACT

Embodiments of the invention include determining whether an individual has autism spectrum disorder or is at risk for developing autism spectrum disorder or at risk for regression of or into autism spectrum disorder. Specific embodiments include the determination of indicative levels of carnitine or other metabolites in carnitine biosynthesis and may include assaying for mutations in TMLHE, including in exon 2, for example. In some cases one can assay for mutations in TMLHE in the absence of biochemical analysis of carnitine biosynthesis metabolites. An individual with deficiency in TMLHE and/or carnitine levels may be administered carnitine, acetylcarnitine, butyrobetaine, or a combination thereof, for example.

This application claims priority to U.S. Provisional Patent Application Ser. No. 61/498,067, filed on Jun. 17, 2011, which is incorporated by reference herein in its entirety.

### STATEMENT REGARDING FEDERALLY SPONSORED RESEARCH OR DEVELOPMENT

This application was made with government support under HD-37283 awarded by NIH and P30HD0240640 awarded by NICHD. The government has certain rights in the invention.

### TECHNICAL FIELD

The field of the present invention concerns medicine, cell biology, molecular biology, and public health, for example. In specific cases, the field of the invention generally concerns diagnosis of autism and/or prevention of the onset of autism for individuals at risk. In particular cases, the field of the invention concerns assaying carnitine biosynthesis and/or genetic detection of defects in a carnitine biosynthesis gene or gene product, such as TMLHE.

### BACKGROUND OF THE INVENTION

The etiology of severe, dysmorphic autism with a male:female ratio of 3.2:1 (Miles et al., 2008) has become increasingly well defined as due to de novo mutations or recent mutations transmitted for a few generations. These mutations include large copy number variants (CNVs), which are detectable by chromosomal microarray analysis in up to 25% of the most severe cases with phenotypes including severe intellectual disability that restricts reproduction (Jacquemont et al., 2006). Disease-causing CNVs are found in ˜10% of patients with intermediate phenotypes with less severe intellectual disability (Sebat et al., 2007; Marshall et al., 2008), and it is common to identify other family members with the pathological CNV. In these cases, penetrance may be incomplete, and the phenotype can be highly variable with diagnoses of intellectual disability, autism, schizophrenia and idiopathic epilepsy seen with the same CNV (van Bon et al., 2009; Sebat et al., 2009; Shinawi et al., 2009). The preceding examples typically represent single locus conditions perhaps with genetic and nongenetic modifier effects. These phenotypes then blend into even more complex genotype-phenotype relationships with evidence for two-hit or two-loci pathogenesis (Girirajan et al., 2010). At the much milder end of the autism spectrum are patients who have speech, have a normal or near-normal IQ, and are non-dysmorphic. This milder population can have as much as an 8:1 male:female ratio (Scott et al., 2002; Kalra et al., 2005), and will be referred to here as non-dysmorphic autism (NDA). This includes the milder portion of the autism spectrum, but NDA patients can have severe cognitive and behavioral phenotypes. The etiology of NDA is much less understood if not completely enigmatic, but the extreme sex ratio may be a strong clue as to the etiology.

There has been discussion of mitochondrial abnormalities in autism as reviewed recently (Rossignol and Frye, 2011), and there are reports of low plasma carnitine values in autism (Gargus and Imtiaz, 2008; Mostafa et al., 2005; Lombard, 1998; Filipek et al., 2004). Carnitine is essential for mitochondrial function where its role is to allow fatty acids to enter mitochondria (Vaz et al., 2002). Although humans can synthesize carnitine, systemic carnitine deficiency can occur under unusual circumstances as when infants were fed soy formulas deficient in carnitine (Slonim et al., 1981). Thus carnitine has been described as a conditionally essential nutrient (Flanagan et al., 2010) and even termed vitamin B_{T}. A genetic form of systemic carnitine deficiency (OMIM 212140) is caused by homozygous loss of function mutations in the SLC22A5 gene which encodes the OCTN2 carnitine transporter. Systemic carnitine deficiency is characterized by cardiac and skeletal myopathy, fatty liver, and life-threatening metabolic aberrations including hypoglycemia, but not typically by autism.

As part of a study examining exon copy number for most of the genes in the genome in simplex autism families, the inventors identified a male with deletion of exon 2 of the TMLHE gene, which is on the long arm of the X chromosome near the boundary of the pseudoautosomal region and encodes 6-N-trimethyllysine dioxygenase. TMLHE encodes the first enzyme in the pathway for carnitine biosynthesis (Vaz and Wanders, 2002), and the enzyme is localized to the mitochondria (Vaz et al., 2002). There are no reports of human loss-of-function mutations (Strijbis et al., 2010), although one instance of deletion of exon 2 in a healthy CEPH male (NA12003) is found in publicly available data (McCarroll et al., 2006). Deletion of exon 2 removes the first methionine codon and the mitochondrial targeting signal, and as shown in this report, results in loss of enzyme activity.

### SUMMARY OF THE INVENTION

In certain embodiments, the present invention is directed to a system, methods, and/or compositons that concern diagnosis of autism, autism regression, low carnitine levels associated with autism, autism regression, or risk of developing autism, and/or identification of individuals at risk for developing at least one symptom of autism. An individual suspected of having autism may have no detectable symptoms, but there may be a family history or general parental concern, for example. Individuals with detectable symptoms of autism include symptoms such as deficiencies in social interactions and relationships; verbal and nonverbal communication; and/or limited interests in activities or play, and specific symptoms are well known in the art. In embodiments of the invention, there is treatment of an individual that is already symptomatic as well as prevention of the onset of autism.

In some embodiments, the present invention concerns a system, methods and/or compositions that concern treatment and/or prevention for an individual with autism, autism regression, low carnitine levels associated with autism, and low carnitine levels that present a risk for autism or regression of autism, such that the treatment may follow determining a level of carnitine or one or more of a carnitine metabolism metabolite and/or that may follow determining whether or not the individual has one or more mutations in TMLHE and/or SLC22A5 and/or SLC6A14 and/or SLC25A45. Exemplary prevention embodiments are useful to prevent the onset of autism, for example in an individual with genetic and/or environmental characteristics that put him or her at risk for developing autism. Some exemplary prevention embodiments are useful to prevent autism regression in an individual that has autism, including that is known to have autism. Some exemplary prevention embodiments are useful to prevent low carnitine levels in an individual at risk for developing autism, such as an individual with one or more certain genetic mutations, an individual with low carnitine levels, and/or an individual with a low intake of meat, particularly beef.

In some embodiments of the invention, an individual is subjected to methods of the invention wherein the individual has certain physical characteristics that renders him or her in need of treatment and/or prevention methods. The individual may have one or more mutations in TMLHE and/or SLC22A5 and/or SLC6A14 and/or SLC25A45. In some aspects of the method it further comprises the step of assaying for a mutation in a carnitine-related gene, such as TMLHE, SCL22A5, SatLC25A45, SHMT1, SHMT2, and/or SLC6A14, or a combination thereof. Others might include at least SLC6A13, the gene encoding HTML aldolase, (HTMLA/ALDH9A1), the gene encoding TMABA-dehydroxygenase (TMABA-DH) and the BBOX1 gene encoding γ-butyrobetaine dioxygenase (γBBD). Also for transport in and out of mitochondria, the CPT1 genes, especially the CPT1C encoding brain-specific CPT1-C, the CPT2 gene, and the CACT gene (SLC25A20).

The individual may have below normal levels of carnitine in the cerebrospinal fluid, blood, and/or urine, for example. The individual may have deficient ratios of particular carnitine metabolites in the cerebrospinal fluid, blood, and/or urine, for example. The individual may be an infant, toddler, child, or adult. The individual may be male or female. The individual may be subjected to a diet low in meat, such as, for example, a vegetarian diet, or the individual may be an infant that has yet to begin solid meats in their diet, for example, to any extent. The individual may have autism or autism regression and may have a particular subtype of autism, such as non-dysmorphic autism, for example. The individual may have a history of regression of autism. The individual may be considered by standard measures to have higher functioning autism, although in other cases the individual may be considered by standard measures to have lower functioning autism. The individual may be considered to have failure to thrive. The individual may have a neuronal carnitine deficiency, and in some embodiments the determination of the neuronal carnitine deficiency is part of the inventive method. In specific embodiments, the carnitine deficiency is in neuronal mitochondria, such as at the synapse. In some embodiments, TMLHE is mutated, which results in autism through the dysfunction of neurons because of toxic accumulation or deficiency of one or more carnitine metabolites.

The individual may be one of a family having two or more boys with autism and the individual may have a mutation in TMLHE and/or SLC22A5 and/or SLC6A14 and/or SLC25A45. In specific embodiments of the invention, an individual is diagnosed with autism yet the autism is not syndromic autism.

In embodiments of the invention, the increased risk of autism is modified by dietary intake of carnitine from birth through the first few years of life.

In some cases, the levels of particular carnitine metabolites are increased in urine and/or plasma, wherein as certain cases the levels of particular carnitine metabolites is decreased in urine and/or plasma, and in particular embodiments these levels are predictive of the dysfunction of the TMLHE gene and are useful to identify individuals with autism. In some embodiments, urine or plasma or CSF is employed for detecting TMLHE and/or SLC22A5 and/or SLC6A14 and/or SLC25A45 deficiencies. In certain embodiments, urine or plasma or CSF is employed for detecting one or more carnitine metabolite levels.

Although in some cases determination of a mutation in TMLHE and/or SLC22A5 and/or SLC6A14 and/or SLC25A45 and/or of a level of one or more carnitine metabolies and/or of obtaining a sample from the individual in question may be performed by the same individual or organization, in other cases the determination(s) is performed by another individual or organization. Samples from the individual suspected of having low carnitine levels, suspected of having a deficiency in carnitine metabolism (including neuronal), and/or suspected of having a mutation in TMLHE and/or SLC22A5 and/or SLC6A14 and/or SLC25A45 may be transported to another party for analysis and/or may be stored under appropriate conditions for later analysis.

In some embodiments of the invention, there is carnitine supplementation in infancy to prevent or reverse autism or any symptoms of autism, including that associated with TMLHE deficiency, for example. In specific embodiments of the invention, heterozygous or other hypomorphic genotypes for systemic carnitine deficiency through mutations in SLC22A5 and/or SLC6A14 and/or SLC25A45 are a risk factor for autism, and that this risk is modified by carnitine supplementation in infancy, in certain embodiments. In aspects to the invention, high male to female ratio in some populations is attributable to sex-specific differences in carnitine metabolism, and at least some autism could be prevented by dietary supplementation of infants' diets with carnitine and/or a carnitine precursor, resulting in abnormalities that are reversible in the early weeks or months after onset of symptoms. In certain aspects to the invention, episodic carnitine deficiency results in regression in autism.

In some embodiments of the invention, at risk infants have low dietary intake of carnitine, impaired synthesis of carnitine or a carnitine metabolite, and/or impaired transport of carnitine or a carnitine metabolite in the GI tract or kidney, including across the BBB; in some cases, such infants have low or normal free plasma carnitine. In some embodiments, infants at risk have low CSF carnitine and/or a high plasma/CSF ratio for free carnitine. In specific embodiments of the invention, males are more susceptible than females because of expression of the SLC6A14 carnitine transporter, which is estrogen-inducible and is on the X chromosome and likely is not be subject to X-inactivation.

In some embodiments of the invention, minor illnesses increase the risk of autism symptoms and precipitate regression, and in particular embodiments the period of greatest danger is associated with an interval of low carnitine intake from the start of solid foods until intake of meats is added. Infants in vegetarian families are at increased risk, in specific embodiments. A secular trend of decreasing intake of beef in the USA over the last three decades and increased incidence of minor illnesses related to group daycare explain an increasing incidence of autism, in certain aspects of the invention.

In certain embodiments of the invention, a single metabolite is measured, although in some cases two or more metabolites are measured. In specific embodiments, a ratio of two metabolites are employed in diagnosis and/or treatment and/or prevention embodiments of the invention. In specific aspects, free carnitine is measured alone or in conjunction with another metabolite. When one or more metabolites are measured, the one or more metabolites may come from the same type of sample (CSF, blood, urine, for example), or it or they may come from another type of sample (CSF, blood, urine, for example) from the same individual. In certain embodiments, the ratio of a plasma metabolite to a CSF metabolite is determined, for example, such as of plasma free carnitine to CSF free carnitine. In some embodiments, the ratio of any metabolites, including plasma free carnitine to CSF free carnitine, is indicative of ability to transport across the blood-brain barrier. CSF levels of one or more metabolites may be measured as described in Mandal et al. (2012), for example, which is incorporated by reference herein in its entirety.

In embodiments of the invention, carnitine levels are important in young non-dysmorphic males. In some embodiments the individual is from about 4 months to 3 years old (or from birth onwards and in certain embodiments prenatally). In specific embodiments a history of regression increases the probability of carnitine level being a factor associated with autism.

In some embodiments of the invention, there are diagnostic methods or treatment methods that include steps of determining level of CSF carnitine metabolites and/or of the plasma to CSF ratio of carnitine metabolites to identify those that will respond to carnitine supplementation and also identifies asymptomatic young infants at risk of autism compared to those that are not at risk. In certain aspects these methods also include analysis of DNA or RNA or carnitine-related genes, such as TMLHE and/or SCL22A5 and/or SLC6A14 and/or SLC25A45 and/or SLC25A45.

In some cases, the plasma levels are normal to slightly lowered depending on carnitine in the diet. As the ratio Plasma:CSF is about 40:1 in certain embodiments, there may be very little active and passive transport of carnitine over the BBB. This indicates that the brain carnitine is primarily derived from biosynthesis. In TMLHE deficiency one would expect significantly lowered carnitine levels and therefore a higher ratio plasma:CSF. In some embodiments, TML is elevated and the HTML/TML ratio is lowered.

In other embodiments, the present invention is directed to a system and method that concern detection of carnitine deficiency in an individual suspected of having the deficiency or known to have the deficiency, for example to determine the severity of the deficiency. In specific embodiments the individual is a child or an infant, although in some cases the individual is an adolescent or adult. In specific cases, the individual is male, although the invention may also be employed for a female. Individuals suspected of having carnitine deficiency may have symptoms such as brain dysfunction, a weakened and enlarged heart (cardiomyopathy), confusion, muscle weakness, and low blood sugar, for example. In certain aspects of the invention, there is testing of individuals with signs of autism, but in other aspects of the invention there is testing if all normal infants, for example as in newborn screening. In some embodiments, children with autism might not have the symptoms associated with severe systemic carnitine deficiency. In specific embodiments, children with autism may or may not have neuronal carnitine deficiency, and at least in certain embodiments this may only be detected by testing spinal fluid.

In specific embodiments, biochemical and/or genetic assays are employed to determine whether an individual has autism or is at risk for developing autism. In particular cases, the individual is at risk for autism regression. In certain cases, the biochemical and/or genetic assays of the invention generally concern carnitine biosynthesis and determination of levels and/or the ability to function of one or more gene products in the carnitine biosynthesis pathway. In some embodiments, newborns or young healthy infants or children are subject to methods of the invention.

Biochemical analysis of carnitine biosynthesis may occur by any suitable means in the art. The analysis may include determination of the levels and/or activity of one or more metabolites of carnitine biosynthesis or enzymes involved therein. In specific embodiments, the biochemical analysis includes carnitine and/or precursors of carnitine in the pathway. Exemplary carnitine biosynthesis metabolites, including 6-N-trimethyl-lysine (TML), 3-hydroxy-TML (HTML), 4-trimethylaminobutyraldehyde (TMABA), 4-N-trimethylaminobutyrate (γ-butyrobetaine, γ-BB), and carnitine. After release of TML by lysosomal protein degradation, this compound is hydroxylated by TML dioxygenase (TMLD, which is encoded by the TMLHE gene), after which the resulting compound is 3-hydroxy-TML (HTML). HTML is cleaved by HTML aldolase, (HTMLA) which uses pyridoxal 5′-phosphate (PLP) as a cofactor, into 4-trimethylaminobutyraldehyde (TMABA) and glycine. Subsequently, TMABA is oxidized by TMABA-dehydroxygenase (TMABA-DH) to form 4-N-trimethylaminobutyrate (γBB). Finally, γBB is hydroxylated by γ-butyrobetaine dioxygenase (γBBD), yielding L-carnitine. Because TMLD is located in mitochondria, TML needs to be transported across the inner mitochondrial membrane into the mitochondrial matrix by means of a transporter. Depending on the subcellular localization of the HTLMA, certain carnitine metabolites (such as HTML or γBB, for example) may need to be transported back to the cytosol (for example, where γBBD is located). In cells that do not contain γBBD, γBB is exported from the cell and imported into tissues (for humans, in liver, kidney and brain) that do express γBBD by means of at least one specific transporter (for example, SLC6A13, but also OCTN2 is capable of γBB transport). These transporters are useful for carnitine biosynthesis and in specific embodiments are included in the context of carnitine biosynthesis, as used herein. Embodiments of the biochemical analysis of carnitine biosynthesis may occur by any one of these components and/or combinations thereof. In specific cases, the assay includes enzyme assays.

Exemplary biochemical means to assay carnitine biosynthesis include (H/UPLC) mass spectrometry, or enzymatic or H/UPLC. In some cases, a labeled standard is employed as an internal control, such as a stable isotope-labeled standard. In some cases, levels of multiple components of carnitine biosynthesis are assayed in a single analytical run. In specific embodiments the ratio of levels of particular components of carnitine biosynthesis are determined. For example, the ratio of HTML/TML, γBB/TML, or (HTML+γBB)/TML is determined. As TML is high, and HTML and γBB are low, in TMLHE deficiency the HTML/TML, γBB/TML and (HTML+γBB)/TML ratios are high in controls and approximate zero in TMLHE-deficient patients. In urine, the HTML/TML and (HTML+γBB)/TML ratio, but not the γBB/TML are useful (the γBB concentration is dependent on carnitine intake and therefore in some cases may be too variable, for example in case of carnitine supplementation). In plasma, the HTML/TML, γBB/TML and (HTML+γBB)/TML ratios are all good markers of TMLHE deficiency, but as the HTML concentration in plasma is very low and γBB concentration is relatively high and constant, γBB/TML and (HTML+γBB)/TML are useful ratios in this matrix, in at least certain embodiments ((patients had a (HTML+γBB)/TML ratio of 0.04/0.05 and the lowest control was 1.4). The equally discriminative γBB/TML ratio in plasma makes it likely that it is useful, for example for bloodspot analysis. In some embodiments, ratios that are not indicative of or sensitive for TMLD deficiency are TML/carnitine, γBB/carnitine, HTML/γBB, and HTML/carnitine, thus in embodiments a ratio carnitine metabolites other than one of these ratios is determined. In specific cases, a certain ratio of carnitine biosynthesis components is indicative of carnitine biosynthesis deficiency and indicative of the need to further investigate if there are deletions in TMLHE gene, for example.

In certain embodiments of the invention, other carnitine biosythesis gene defects are employed in the invention. In a case of deficiency of the mitochondrial transporters involved in the shuttling of TML and HTML (and/or TMABA) the same ratios described for TMLD deficiency are applicable.

In a case of HTMLA deficiency, however, the γBB/HTML ratio is useful as HTML accumulates and γBB is deficient (i.e. a low ratio in HTMLA deficiency).

In particular embodiments, the γBB/HTML ratio is useful to evaluate TMABA-DH deficiency (as TMABA is unlikely to accumulate, but if it does the γBB/TMABA ratio is better, in at least certain cases), again low ratio (high HTML, low γBB) indicates deficiency.

In a case of γBBD deficiency the carnitine/γBB ratio is useful (at least in plasma) as γBB will be high and carnitine will be lower or the same as in control individuals.

In certain embodiments, in a case of deficiency of SLC6A13 or any other plasma membrane γBB transporter the carnitine/γBB ratio will, as with γBBD deficiency, also indicate deficiency in both urine and plasma (or in CSF, as SLC6A13 provides the brain with butyrobetaine, in at least certain aspects).

In some cases, a certain ratio of carnitine biosynthesis components is indicative of the presence of autism or a risk for development of autism or a risk for regression with autism.

In some embodiments of the invention, derivatives of TML (for example, 2-N-acetyl-6-N-trimethyllysine, 2-Oxo-6-trimethylammoniohexanoate and 5-trimethylammoniopentanoate) may be utilized as indicators of TMLD deficiency, for example, and, in certain cases, of the risk or presence of autism or regression thereof. In some cases, the metabolic effects of γBB deficiency are associated with diagnosis or risk of autism, as this metabolite has strong structural resemblance to known neurotransmitters (for example, acetylcholine and gamma-aminobutyric acid (GABA)). In certain cases, deficiency of γBB contributes to the autism phenotype and, thereby, butyrobetaine is diagnostic or prognostic in autism.

Genetic analysis of TMLHE may be utilized in certain embodiments of the invention. The analysis may include determination of one or more mutations in the gene. The mutation(s) may be present anywhere in the gene including coding or non-coding sequence, for example, promoter, intron, exon, 5′UTR, 3′ UTR, and so forth. In specific embodiments the mutation results in a defective translated gene product compared to wild-type. In particular aspects, the mutation is a point mutation, frameshift mutation, insertion, deletion, inversion, and so forth. In specific embodiments, the mutation is a deletion of at least one base pair. The mutation may be in any exon, in specific cases, but in particular embodiments it is in exon 2. In alternative embodiments, the one or more mutations are located in exons 1, 2, 3, 4, 5, 6, 7, 8, or 9. In some cases, part of an exon is deleted, although in other cases an entire exon is deleted, such as exon 2, for example.

In one embodiment of the invention, one or more mutations are assayed for that removes or inactivates the mitochondrial targeting signal.

Any suitable method in the art may be employed for genetic analysis of TMLHE. The gene or its gene product, including its mRNA, may be assayed, in certain embodiments. In specific embodiments, RT-PCR is employed to avoid the interference of pseudogenes within the genomic copy of TMLHE.

An exemplary TMLHE sequence is provided at GenBank® Accession No. NG_{—}021318, which is incorporated by reference herein in its entirety. Such an exemplary sequence is useful to identify regions of the TMLHE sequence suitable for primers for amplification of certain regions of the sequence, such as for determination of deletions of part or all of the sequence. The mutation(s) may be identified by size differences compared to a wild-type control, by sequence of an amplified region, or both, for example. The primers may target sequences within an exon or outside an exon, or one of each kind may be employed. The invention includes a set of primers that can detect the presence of a deletion in exon 2.

Mutations in other genes may be identified by routine methods in the art, including for SLC25A45 (GenBank® Accession Nos. NM_{—}182556 or NM_{—}001077241, which are different transcript variants); SCL22A5 (GenBank® Accession No NM_{—}003060), SLC6A14 (GenBank® Accession No. NM_{—}007231), SHMT1 (GenBank® Accession No. BC007979), SHMT2 (BC01191), SLC6A13 (GenBank® Accession No. NM_{—}016615, NM_{—}001190997 or NM_{—}001243392, which are different transcript variants), HTMLA, ALDH9A1 (GenBank® Accession No. NM_{—}000696), TMABA-DH, BBOX1 (GenBank® Accession No. BC011034), CPT1-C (NM_{—}001136052 NM_{—}001199752; NM_{—}001199753, and NM_{—}152359 that are transcript variants), CPT2 (GenBank® Accession No. NM_{—}000098), and SLC25A20 (GenBank® Accession No. NM_{—}000387). All GenBank® sequences are incorporated by reference herein in their entirety.

In certain aspects of the invention, a sample from an individual is assayed for analysis of carnitine biosynthesis and/or mutation in TMLHE. The individual may be an individual with no obvious symptoms of autism spectrum disorder. Such individuals may present one or more symptoms of autism spectrum disorder following environmental factors, such as low levels of carnitine in the diet and/or response to an infection or inflammation. The response to an infection or inflammation may include fever, in some cases. In specific embodiments, the individual has mitochondrial dysfunction, which may have symptoms such as poor growth, loss of muscle coordination, muscle weakness, visual problems, hearing problems, learning disabilities, mental retardation, heart disease, liver disease, kidney disease, gastrointestinal disorders, respiratory disorders, neurological problems, autonomic dysfunction, and dementia. In some cases, the infection or inflammation is an expected event, such as from exposure to a vaccination.

It is useful in at least certain embodiments of the invention to biochemically assay carnitine levels, given that in about 10% of individuals may lack a detectable mutation in TMLHE.

In certain embodiments of the invention, instead of or in addition to a carnitine biosynthesis precursor or enzyme being assayed, another carnitine-related compound is assayed, such as transporters of carnitine and/or its precursors, including SLC22A5, SLC6A13, SLC6A14 and SLC25A45.

Samples from the individual are assayed for biochemical and/or genetic information useful to determine if the individual has autism or is at risk of developing autism or developing regression in autism. The sample may be of any suitable kind, although in specific embodiments it includes blood, plasma, urine, cerebrospinal fluid, biopsy, saliva, cheek scrapings, and so forth. The sample may include venous blood or dried blood spots, in specific embodiments. In some cases the sample includes cells, such as white blood cells or cultured lymphoblasts, for example 1n an embodiment both biochemical and genetic analysis is performed on material from a single sample (or aliquots from a single sample), e.g., single blood sample, from the individual.

In certain embodiments of the invention, a metabolic assay of carnitine biosynthesis includes diagnosis of carnitine biosynthesis deficiency. Such a determination may be followed by analysis of TMLHE defects, including deletion in exon 2, for example. In specific cases, a sample from the individual may be subjected to enzymatic assay of one or more enzymes in carnitine biosynthesis.

Individuals may be supplemented with carnitine, acetyl-carnitine, propionylcarnitine and/or γBB following analytical methods of the invention, in particular embodiments of the invention. The individuals may be known to have carnitine deficiency and/or autism, the individual may be subject to environments with frequent transmission of pathogens, such as a daycare, the individual may have low carnitine levels in the diet, the individual may be known or suspected to have mitochondrial dysfunction, the individual may be subject to a vaccine, and/or the individual may be known to have a defect in TMLHE. The supplemental carnitine, acetyl-carnitine, propionylcarnitine and/or γBB may occur at any time, although in specific embodiments the supplemental carnitine, acetyl-carnitine, propionylcarnitine and/or γBB may be consumed by the individual the same day of exposure to pathogen or exposure to a vaccine, for example, or the consumption may begin within less than 1, 1, 2, 3, 4, 5, 6, 7, 8, 9, 10, 11, 12, 13, or 14 days (for example) following exposure to pathogen or exposure to a vaccine. Carnitine, acetyl-carnitine, and/or butyrobetaine may be consumed daily following exposure to pathogen or exposure to a vaccine for less than 1, 1, 2, 3, 4, 5, 6, 7, 8, 9, 10, 11, 12, 13, or 14 days, for example. The vaccine may be a single vaccine or a combination vaccine. The vaccine may include live viruses or killed viruses, for example. In specific embodiments of the invention, 100 mg/kg/day or less is an effective amount provided to the individual of carnitine, acetyl-carnitine, propionylcarnitine and/or γBB.

In aspects of the invention, food and/or drink is supplemented with carnitine, acetyl-carnitine, propionylcarnitine and/or γBB. The supplementation may be increased in baby formula over that presently available. In some cases, the supplementation occurs in solid baby food, including baby fruits, cereals, and/or vegetables, for example.

In some embodiments, there is a method of preventing regression or development of autism in an individual that has or is at risk for mitochondrial dysfunction, comprising the step of administering carnitine, acetylcarnitine, propionylcarnitine γBB, or a combination thereof to the individual prior to, during, and/or after exposure of the individual to an event that results in infection or inflammation, wherein the regression or development of autism is prevented in the individual. In specific embodiments, the event is the onset of fever. In certain cases, the event comprises the administration of a vaccine, such as a vaccine for measles/mumps/rubella, chickenpox, diphtheria, hepatitis A, hepatitis B, anthrax, Haemophilus influenzae type b, Human Papillomavirus, influenza, Japanese encephalitis, Lyme disease, meningococcal vaccine, pertussis, polio, pneumococcus, rabies, rotavirus, Herpes Zoster, smallpox, tetanus, turberculosis, typhoid, yellow fever, or a combination thereof, for example. The individual may be a child or adolescent. The carnitine, acetylcarnitine, propionylcarnitine γBB, or a combination thereof may be delivered orally. In some cases, the individual has neuronal carnitine deficiency.

In certain embodiments of the invention, methods comprise obtaining a sample, isolating genomic DNA from the sample, and analyzing TMLHE nucleic acid in the sample.

In embodiments of the invention, there is a method of identifying an individual with autism spectrum disorder or at risk for developing autism spectrum disorder or regression thereof, comprising the step of assaying for impaired carnitine biosynthesis in a sample from the individual, wherein when there is impaired carnitine biosynthesis, a sample from the individual is assayed for a mutation in a trimethyllysine dioxygenase (TMLHE) polynucleotide. In a specific embodiment, analysis for level of a carnitine metabolite and an analysis for a mutation in the TMLHE polynucleotide are performed on the same sample (or on aliquots of a single sample) from the individual.

In certain embodiments of the invention, impaired carnitine biosynthesis comprises reduced carnitine levels compared to a reference, e.g., a reference that is a function of levels from an unaffected individual. In at least specific cases, the assay for impaired carnitine biosynthesis comprises measuring the levels of more than one metabolite of carnitine biosynthesis.

In some embodiments of the invention, a ratio of two metabolites is determined, including two carnitine metabolites, for example. In specific embodiments, the ratio of 3-hydroxy-TML (HTML) to a second metabolite, e.g., carnitine metabolite, is determined, and in certain embodiments, the ratio of 6-N-trimethyl-lysine (TML) to a second metabolite, e.g., carnitine metabolite, is determined. In particular cases, the ratio of 3-hydroxy-TML (HTML) to 6-N-trimethyl-lysine (TML) is determined. In particular aspects, the ratio of γ-butyrobetaine dioxygenase (γBB) to a second metabolite, e.g., carnitine metabolite, is determined, such as the ratio of γBB to TML, for example. In some cases, the particular value of more than one ratio in combination is indicative of the presence of autism or the risk for developing autism or regression thereof and, optionally, also the presence of a mutation in TMLHE or another carnitine biosynthesis gene or transporter. For example, the particular value of both TML/HTML and TML/γBB are considered indicative of the presence of autism or the risk for developing autism or regression thereof.

In some embodiments of the methods, the mutation in TMLHE is in exon 2, such as a deletion including wherein the entire exon 2 is deleted. In some aspects, the assay for a mutation in TMLHE comprises analysis for the mutation in the TMLHE mRNA, such as by RT-PCR, for example.

In certain embodiments of methods of the invention, the method occurs prior to exposure of the individual to infection or inflammation, and in certain aspects the method occurs prior to vaccination of the individual. Exemplary vaccines include the vaccine for measles/mumps/rubella, chickenpox, diphtheria, hepatitis A, hepatitis B, anthrax, Haemophilus influenzae type b, Human Papillomavirus, influenza, Japanese encephalitis, Lyme disease, meningococcal vaccine, pertussis, polio, pneumococcus, rabies, rotavirus, Herpes Zoster, smallpox, tetanus, turberculosis, typhoid, yellow fever, or a combination thereof, for example.

In certain embodiments, methods of the invention occur prior to the onset of fever.

In some aspects of the invention, when the individual is determined to have deficient carnitine levels, the individual is provided with an effective amount of carnitine, acetylcarnitine, propionylcarnitine, butyrobetaine, or a combination thereof, for example.

Individuals exposed to methods and/or compositions of the invention may be an infant, child or adolescent, although in some cases the individual is an adult. In at least some cases, the individual has neuronal carnitine deficiency.

In certain aspects of the invention, the individual is provided an effective amount of carnitine, acetylcarnitine, propionylcarnitine, butyrobetaine, or a combination thereof, for example delivered orally.

In some cases, the sample is obtained from the individual, and in particular aspects the sample is blood, urine, or cerebrospinal fluid, for example. The sample may comprise cells.

In some aspects of the methods of the invention, the sequence of at least one nucleotide position in a second carnitine metabolism gene, e.g., a gene disclosed herein, is determined.

In one embodiment of the invention, there is a method of evaluating an individual, e.g., identifying an individual with autism spectrum disorder or at risk for developing autism spectrum disorder or regression thereof, comprising the step of measuring a ratio of metabolites of carnitine biosynthesis, wherein when there is determination of a particular ratio of the metabolites, the individual has autism spectrum disorder or is at risk for developing autism spectrum disorder or regression thereof. In some cases, the ratio is further defined as the ratio of HTML to TML or γBB to TML or both. In specific embodiments, the sequence of at least one nucleotide position in a carnitine metabolism gene, e.g., a gene disclosed herein, is determined.

Some embodiments of the invention further comprise the step of assaying for a mutation in TMLHE. The mutation may be a deletion in one or more of the exons of TMLHE, such as exon 2 of TMLHE.

In some embodiments of the invention, the sequence of at least one nucleotide position in a second carnitine metabolism gene, e.g., a gene disclosed herein, is determined.

In some embodiments of the invention, there is a method of evaluating an individual, e.g., identifying an individual with autism spectrum disorder or at risk for developing autism spectrum disorder or regression thereof, comprising the step of assaying for a mutation in exon 2 of a TMLHE polynucleotide. In some embodiments of the invention, the methods further comprises the step of obtaining a sample from the individual.

Certain embodiments of the invention include, for example, optionally acquiring nucleic acid from said subject; determining if there is a mutation in exon 2 of the TMLHE gene (which in some cases is a TMLHE polynucleotide, including an mRNA expressed from the gene), thereby evaluating said individual. In some cases, the mutation in exon 2 of the TMLHE gene is positively correlated with autism spectrum disorder or at risk for developing autism spectrum disorder or regression thereof. In some aspects of the invention, responsive to said determination, there is a) assigning a risk classification to said individual; b) selecting said individual for further testing, e.g., sequence analysis of a second gene associated with carnitine metabolism, e.g., a carnitine metabolism gene described herein; c) selecting said individual for further testing, e.g., analysis of carnitine, or a carnitine metabolite, e.g., a carnitine metabolite described herein; d) selecting said individual for administration of a dietary carnitine supplement, e.g., an individual having or at risk for developing fever. In at least some aspects, the method further comprises performing one of steps a-d. In some aspects of the invention, acquiring comprises removing tissue from the individual.

In some aspects of the invention, determining comprises a method that imparts a physical change to said nucleic acid or another component in a sample comprising said nucleic acid, the making or breaking of at least one chemical bond, performing a chemical reaction, e.g., a sequencing reaction, or separating or purifying a first component from a second component in the a sample comprising said nucleic acid. In a specific embodiment, determination comprises determining the sequence (wherein sequence can mean the identity, or presence or absence) of at least one nucleotide position in an exon 2 lesion, e.g., an exon 2 mutation described herein, e.g., an exemplary mutation described in Tables S1, S3 or S4 (for example) herein.

In some embodiments of the invention, there is a method of evaluating an individual, e.g., selecting an individual for further evaluation or for treatment, comprising, optionally, acquiring a value for a parameter correlated with impaired carnitine metabolism; responsive to said value, e.g., if said value is at or below a predetermined reference value, determining if there is a mutation in a carnitine metabolism gene, e.g., a carnitive metabolism gene described herein, e.g., in the TMLHE gene, e.g., in exon 2 of the TMLHE gene, thereby evaluating said individual. In some embodiments, the mutation in exon 2 of the TMLHE gene is positively correlated with autism spectrum disorder or at risk for developing autism spectrum disorder or regression thereof. In at least some cases, an analysis for level of a carnitine metabolite and an analysis for a mutation in exon 2 of the TMLHE gene are performed on the same sample (or on aliquots of a single sample) from the individual. In certain embodiments, the parameter is a function of the level of more than one metabolite of carnitine biosynthesis. In certain aspects, a parameter is a function of the ratio of two metabolites, such as a ratio of 3-hydroxy-TML (HTML) to a second metabolite, e.g., carnitine metabolite; in certain cases, there is a ratio of 6-N-trimethyl-lysine (TML) to a second metabolite, e.g., carnitine metabolite.; in specific embodiments, the ratio is the ratio of γBB to a second metabolite, e.g., carnitine metabolite. In some cases, a parameter is a function of the ratio of 3-hydroxy-TML (HTML) to 6-N-trimethyl-lysine (TML). In at least specific cases, a parameter is a function of the ratio of γBB to TML.

In one embodiment of the invention, there is a method of generating a report for an individual, comprising, acquiring, e.g., by a method that comprises measuring a carnitine metabolite in a sample, a value for a parameter correlated with impaired carnitine metabolism; acquiring, e.g., by a method that comprises sequencing nucleic acid in a sample, the sequence of at least one nucleotide position in in exon 2 of the TMLHE gene (such as a polynucleotide); generating a report, e.g., comprising a computer readable, or a written report, by inserting a value for impaired carnitine metabolism and inserting a value for the presence of a mutation in exon 2 of the TMLHE gene (such as a polynucleotide) into a record, thereby generating a report for an individual. In some aspects, the method further comprises transmitting a report to a health care provider, third-party payor (e.g., an insurance company, employer, or governmental entity), or relative, e.g., parent of said individual, for example.

In particular embodiments of the method, there is a method of assigning a risk class to an individual, comprising, acquiring, e.g., by a method that comprises measuring a carnitine metabolite in a sample from said individual, a first value (e.g., a numerical or non numerical value) that is correlated with impaired carnitine metabolism; acquiring, e.g., by a method that comprises sequencing nucleic acid in a sample from said individual, a second value (e.g., a numerical or non numerical value) which is correlated to sequence of at least one nucleotide position in in exon 2 of the TMLHE gene; responsive to said first and second value, classifying the individual for risk, thereby assigning a risk class to said individual. In a specific embodiment, the risk comprises risk of an unwanted response to fever, although in some cases the risk comprises risk for developing autism spectrum disorder or regression thereof. In certain embodiments, the risk comprises risk for developing autism spectrum disorder or regression thereof as a result of fever, e.g. fever from vaccination. In a specific embodiment, a first value is a function of more than one metabolite of carnitine biosynthesis, and in some cases, a first value is a function of the ratio of two metabolites. In at least some cases, there is a ratio of 6-N-trimethyl-lysine (TML) to a second metabolite, e.g., carnitine metabolite; in specific embodiments, the ratio is the ratio of γBB to a second metabolite, e.g., carnitine metabolite. In some cases, a parameter is a function of the ratio of 3-hydroxy-TML (HTML) to 6-N-trimethyl-lysine (TML). In at least specific cases, a parameter is a function of the ratio of γBB to TML.

In some embodiments, there is a method of treating an individual having autism or at risk of developing autism or autism regression and in need of determination of need of carnitine supplementation, comprising the step of determining the level of one or more carnitine metabolites in a sample from the individual and when the level of the one or more carnitine metabolites is abnormal the individual is provided with an effective amount of carnitine. In a specific embodiment, the individual is an asymptomatic infant at risk of developing autism, although in some embodiments the individual has non-dysmorphic autism.

In some aspects of the method it further comprises the step of assaying for a mutation in a carnitine-related gene, such as TMLHE, SCL22A5, and/or SLC6A14 and/or SLC25A45, or a combination thereof.

Samples from the individual may be obtained by routine methods, and in certain aspects the sample is cerebrospinal fluid, plasma, or urine. In some embodiments, the ratio of one carnitine metabolite to another carnitine metabolite is determined. In certain methods, they are further defined as determining the plasma to CSF ratio of one or more carnitine metabolites. In particular cases, the individual has at least one other sibling that has autism or has abnormal carnitine levels in the CSF, plasma, or urine, and in certain embodiments the sibling(s) is a male.

In embodiments of the mention, autism is caused by simple dietary deficiency, genetic impairment of carnitine transport particularly across the blood-brain barrier, and defects of endogenous synthesis of carnitine as in TMLHE deficiency.

In some embodiments, there is a method of achieving a desired level of carnitine in an individual in need thereof, said method comprising the steps of determining a level of at least one carnitine metabolite from a sample from an individual that has autism or is at risk of developing autism and that has a defect in a carnitine-related gene; and providing an effective amount of a carnitine metabolite to the individual. In specific embodiments, the individual has non-dysmorphic autism. In some cases, the carnitine-related gene is TMLHE, SCL22A5, and/or SLC6A14 and/or SLC25A45, or a combination thereof. In particular embodiments, the sample is cerebrospinal fluid, plasma, or urine. In certain aspects, the ratio of one carnitine metabolite to another carnitine metabolite is determined. In some aspects, the method is further defined as determining the plasma to CSF ratio of one or more carnitine metabolites. In specific cases, the individual has at least one sibling that has autism or has abnormal carnitine levels in the CSF, plasma, or urine or has a defect in a carnitine-related gene.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1. Carnitine Biosynthesis Pathway and Structure and Gene Variations Found in TMLHE (A) Carnitine homeostasis in man. Schematic overview of carnitine homeostasis: carnitine is synthesized in four enzymatic steps. After release of TML by lysosomal protein degradation, this compound is hydroxylated by TMLD producing HTML. HTML is cleaved by HTML aldolase (HTMLA) into 4-trimethylaminobutyraldehyde (TMABA) and glycine. Subsequently, TMABA is oxidized by TMABA-dehydroxygenase (TMABA-DH) to form 4-N-trimethylaminobutyrate (γBB). Finally, γBB is hydroxylated by γ-butyrobetaine dioxygenase (γBBD), yielding L-carnitine. Because TMLD is located in mitochondria TML needs to be transported across the inner mitochondrial membrane into the mitochondrial matrix by means of a transporter. Depending on the subcellular localization of the HTLMA (perhaps in the cytosol), HTML or γBB need to be transported back to the cytosol (where γBBD is located). In cells that do not contain γBBD, γBB is exported from the cell and imported into tissues (in humans, liver, kidney and brain) that do express γBBD by means of at least one specific transporter, SLC6A13, in certain aspects. Carnitine is transported by OCTN2 and other lower affinity transporters.

(B) Array CGH showing eight exonic deletions in TMLHE with the relative location of TMLHE exons 2-6 aligned above the array CGH plots (GRCh37/hg19 assembly; see World Wide Web site). Horizontal axis, chromosome position; vertical axis, log 2 ratio of array signal. Semi transparent filled boxes on CGH plots highlight the regions of aberration; all samples have deletions involving exon 2, and most have a separate deletion in intron 1 (red circle). All samples are autism probands with identifiers found in Tables 51 and S6.

(C) Twenty-four unrelated individuals with deletions involving exon 2 of TMLHE are mapped. Deletion coordinates were determined by array CGH unless otherwise specified. White arrowheads indicate the continuation of the deletion for SSC 13928.p1. NA 12003 is an unaffected individual from (McCarroll et al., 2006), whose deletion was better characterized in this study. BPR is an unaffected individual.

(D) Diagram of gene structure.

(E) Mother (HI0689) and her unaffected son (HI0692) from AGRE family AU 0177 have an R241Q (G to A nucleotide) missense mutation. The two affected males (HI0690 and HI0691) and the father (HI0688) do not have the mutation.

Fa, father; P1, proband; SSC, Simons Simplex Collection; HI, AGRE individuals; #, individual first described in Celestino-Soper et al. unpublished data; NT Ref., nucleotide sequence of reference; AA Ref, amino acid sequence of reference. See also Tables 51 and S2.

FIG. 2. TMLHE Exon 2 Deletions Produce Stable mRNA.

(A) RT-PCR from lymphoblast cell lines of AGRE family AU 0177 showed the presence of a strongly expressed transcript containing exon 2 in all cases except for HI0690 (affected), who has deletion of exon 2. HI0689 (mother) is heterozygous for the deletion. Low level of exon 2 skipping is seen in the unaffected individuals (HI0688 and HI0692). Arrows represent primers.

(B) Quantitative RT-PCR from lymphoblast cell lines of AGRE family AU 0177 and from BPR individuals normalized to β-actin (endogenous control) and unaffected father HI0688. All samples show detection of the exon 5-6 product. Individuals with hemizygous exon 2 deletion (HI0691 and BPR664) show undetectable TMLHE exon 1-2 product, compared to HI0688. Vertical axis, relative quantification or ΔΔCt (see methods), horizontal axis, TMLHE primer assay and samples tested.

nl, normal; dl, deletion, hGAPDH, human GAPDH control.

FIG. 3. Genetic and Enzymatic Characterization of Hemizygous Deletion of Exon 2.

(A) Upper panel: PCR results for the AU 0177 family showing the deletion in the two affected brothers (1 and 2) and in the mother (3) but not in the father (4), unaffected maternal half brother (5), or unaffected controls (C1 and C2). There is bias of amplification in the mother so that the normal band is faint.

(B) TMLD activity measured in lymphoblast homogenates and western blot analysis of 3 families with exon 2 deletion.

(C) Upper panel: TMLD activity and western blot analysis of two individuals with exon 2 deletion (P1, HI0690; P2, BPR664) and 3 controls (C1-C3). Purified TMLD (pTMLD) is used as positive control. Lower panel: Western blot analysis of two individuals (P1 and P2) with (+) or without (−) addition of purified TMLD, showing the complete absence of protein in cases of exon 2 deletion and confirmation of the identity of the immunoreactive material as TMLD.

(D) Left: TMLD activity measured in lymphoblast homogenates from several autism males. Right: TMLD activity measured in lymphoblast homogenates from male controls. BPR, local unaffected controls; NA 12003 is an unaffected individual. Asterisk: TMLHE exon 2 deletion. Dollar sign: E287K.

(E) TMLD activity measured in cerebellum homogenates from controls and autism probands. See also Tables S4 and S5.; Nl, normal; dl, deletion; fa, father; mo, mother; p1, proband.

FIG. 4. Increased TML and Decreased HTML and γBB in patients with TMLHE Exon 2 Deletions.

Concentrations of carnitine biosynthesis intermediates in urine (top panel) and plasma (middle panel), of two patients (1: HI0690; 2:HI0691) with exon 2 deletion compared to controls. Lower panel depicts a box and whiskers plot showing the diagnostic potential of the (HTML+γBB)/HTML ratio as an indicator of TMLHE deficiency. Patients have a very low ratio. Black square represents the mean of the controls, whiskers show minimum and maximum value of the control group.

TML, 6-N-trimethyllysine; HTML, 3-hydroxy-6-N-trimethyllysine; γBB, γ-butyrobetaine. Mean±SD.

FIG. 5. Analysis of DNA Methylation in Autism and Control Males and Females

Data are shown for three genes as indicated. AVG-Beta values for individual CpGs are shown on the vertical axis (% methylation). AVG-Beta value of 0-0.2 indicates unmethylated state whereas a value from 0.8-1.0 indicates fully methylated state. Values between 0.4-0.6 indicate composite methylation as seen in case of X-linked genes in females. Map information for the probes studied to determine the level of methylation is given on the horizontal axis. Unaffected males (n=36); unaffected females (n=9); autism males (n=24); autism females (n=4).

FIG. 6. Increased TML and decreased HTML and γBB in patients with TMLHE exon 2 deletions. (A) Box and whisker plot of carnitine biosynthesis intermediates in plasma of seven patients with exon 2 deletion (HI0690, HI0691, SSC 13928.p1, SSC 13489.p1, SSC 11000.p1, SSC 11229.p1, and SSC 11680.p1) compared to controls. (B) Box and whisker plot showing the diagnostic potential of the (HTML+γBB)/HTML ratio as an indicator of TMLHE deficiency. All seven patients have a very low ratio. Black square represents the mean of the controls, whiskers show minimum and maximum value of the control group.

### DETAILED DESCRIPTION OF THE INVENTION

### I. Exemplary Definitions

As used herein the specification, “a” or “an” may mean one or more. As used herein in the claim(s), when used in conjunction with the word “comprising”, the words “a” or “an” may mean one or more than one. As used herein “another” may mean at least a second or more. Furthermore, as used herein, the terms “including”, “containing”, and “having” are open-ended in interpretation and interchangeable with the term “comprising”.

The term “acquiring”, as used herein, can comprise a method that imparts a physical change to a sample. This is sometimes referred to herein as directly acquiring and includes, e.g., the making or breaking of chemical bond, e.g., a covalent bond, performing a chemical reaction, e.g., a sequencing reaction or an enzymatic analysis, separating or purifying a first component from a second component in the sample, or contacting the sample with a reagent, e.g., a detection reagent, e.g., an antibody. In other embodiments, the term is sometimes referred to herein as indirectly acquiring, the acquired entity, e.g., a value or a sample, is obtained without the performance of a physical change to the sample, e.g., a value is obtained from another who has performed the direct acquisition.

The term “autism spectrum disorder” as used herein refers to a variety of psychological conditions characterized by widespread abnormalities of social interactions and communication, including repetitive behavior and limited interests.

The term “carnitine metabolite” as used herein refers to any metabolite in the carnitine biosynthesis pathway, including carnitine.

“Effective amount,” “Therapeutically effective amount” or “pharmaceutically effective amount” means that amount which, when administered to a subject or patient for treating a medical condition, is sufficient to effect such treatment for the disease. In specific embodiments for carnitine deficiency or deficiency of one of its biosynthetic precursors, the amount of the compound is the amount that when administered to the individual is sufficient to detectably raise levels of carnitine or one of its biosynthetic precursors.

In general embodiments of the invention, there are novel genes mutated in autism. In certain embodiments of the invention, there is a novel and common X-linked inborn error of carnitine biosynthesis associated with autism, for example associated with neuronal carnitine deficiency in autism. In particular, the invention discloses utilization of array CGH with genome-wide, exon by exon coverage, in a specific case to screen 100 simplex families from the Simons Simplex Collection. One male in the first 100 trios and then numerous other males with autism were found to have inherited deletions of exon 2 of the TMLHE gene on the X-chromosome leading to the delineation of a novel inborn error of metabolism. Deletions of exon 2 are null mutations, in this particular examples, and were recurrent with slightly different boundaries in each family. TMLHE encodes the first enzyme in the pathway for biosynthesis of carnitine, and deficiency is characterized by increased TML and decreased HTML and γBB in plasma, urine, and brain. The Examples show herein that TMLHE deficiency is at least 20 times more common than PKU in males (1 in ˜430), and deletion of exon 2 was 3.7 times more common in probands of male-male affected sib pairs with autism compared to control males (P=0.012). TMLHE deficiency is present in 0.5-1% of autism, and the penetrance of 20-40% in US males may be dependent on carnitine intake during infancy.

In particular aspects of the invention, TMLHE deficiency is extremely common in “normal” males, although subtle phenotypes may occur in these individuals. Because TMLHE is highly expressed in Purkinje cells and hippocampal neurons, and carnitine is essential for mitochondrial function at the synapse, in specific aspects of the invention a neuronal carnitine deficiency is a risk factor for autism. In specific aspects, the fraction of non-dysmorphic autism (NDA) associated with neuronal carnitine deficiency is limited to TMLHE deficiency or could be much greater in certain embodiments wherein nutritional carnitine deficiency was a common associated risk factor for NDA. If males are somehow more susceptible than females to impairment through neuronal carnitine deficiency, this could explain the up to 8:1 male:female ratio in NDA and/or the putative increasing in the frequency of autism and be a common risk factor for autism. Autism associated with TMLHE deficiency or with nutritional carnitine deficiency or deficiency of one of its biosynthetic precursors (such as γBB) may be preventable or partially reversible using carnitine, acetylcarnitine, or γBB dietary supplementation. Carnitine supplementation in young, non-dysmorphic males with autism with and without TMLHE deficiency has clinical benefit, in particular aspects. In certain embodiments, autism associated with TMLHE deficiency is preventable or partially reversible using carnitine, acetylcarnitine, propionylcarnitine or γBB dietary supplementation, for example.

### II. Biochemical Analysis of Carnitine Deficiency

In embodiments of the invention, an individual known to have autism or carnitine deficiency or suspected of having carnitine deficiency or suspected of having autism has a sample measured for carnitine metabolism deficiency. In practice, in specific embodiments, the individual is tested for levels of carnitine that are lower than normal levels, although in particular aspects the individual is instead tested for levels of a component of the carnitine biosynthesis pathway, which may be referred to as a carnitine metabolite. A carnitine metabolite is defined as a substance that is produced during carnitine metabolism or that takes part in carnitine metabolism. In particular aspects of the invention, increased TML and decreased HTML and γBB in plasma, urine, and/or brain is associated with carnitine deficiency and/or autism and is a direct or indirect measurement of carnitine deficiency.

In certain aspects of the invention, more than one carnitine metabolite is assayed for its level, and the levels of the more than one carnitine metabolite are configured for a diagnostic output. In specific cases, the ratio of one carnitine metabolite to another is compared to a wild type control or known value for a normal ratio of the two carnitine metabolites. A difference in value is considered indicative of the individual as having carnitine deficiency and/or autism. In some cases if the ratio is greater than normal, the individual has carnitine deficiency and/or autism. In other cases if the ratio is lower than normal, the individual has carnitine deficiency and/or autism. In case of TMLHE deficiency, for the ratios HTML/TML, γBB/TML and (HTML+γBB)/TML, lower than normal levels are indicative of carnitine deficiency and/or autism

The levels of carnitine biosynthesis metabolites are known in the art. For example, Vaz et al. (2002) refers to the concentration of TML in plasma as being relatively constant in human, ranging from 0.2 to 1.3 μM. In humans, the level of γBB in urine is low (approx. 0.3 μmol·mmol creatinine⁻¹). In humans, the concentration of plasma carnitine is age- and sex-dependent, increasing during the first year of life (from approx. 15 μM to approx. 40 μM), and remains the same for both sexes until puberty. From puberty to adulthood, plasma carnitine concentrations in males increase to a level that is considerably higher than those in females (50 μM compared with 40 μM) (see Vaz et al., 2001).

In certain aspects, any suitable method may be employed to determine levels of carnitine metabolites. In specific cases, samples may be deproteinized, such as by using a Amicon Ultra 30-kDa filter, for example. Urine samples may be directly derivatized, such as with methyl chloroformate. Carnitine metabolites, such as TML, HTML, carnitine and γ-BB, may be quantified using ion-pair UPLC-tandem mass spectrometry as previously described (Vaz et al., 2002), for example. In certain cases, the analytes are separated by ion-pair, reversed-phase HPLC and detected online by electrospray tandem mass spectrometry. Stable-isotope-labeled reference compounds may be used as internal standards.

Carnitine metabolites may be measured enzymatically, by HPLC after derivatization, for example using 4′-bromophenacyl trifluoromethanesulfonate but generally mass spectrometry with or without prior H/UPLC separation and/or extraction/purification procedure is used.

In alternative embodiments, carnitine deficiency is assayed by measuring the enzyme activity of one or more enzymes involved in carnitine metabolism. For example, activity of TML dioxygenase (TMLD) may be measured by providing a suitable reaction mixture and suitable homogenate (See Example 7). Following an appropriate reaction time, the reaction is terminated with a ZnCl₂ solution containing particular internal standards ([²H₉]HTML, 140 pmol [²H₉]TML), followed by centrifugation to separate the metabolites from the enzymes. The HTML produced following derivatization of the filtrate with methylchloroformate was quantified using ion-pair UPLC-tandem mass spectrometry.

For HTMLA, for example, which catalyses the aldolytic cleavage of HTML into TMABA and glycine, one can measure its activity by using radiolabelled hydroxyl-TML. Like many aldolases, in some embodiments HTMLA uses pyridoxal 5′-phosphate, a derivative of pyridoxine (vitamin B6), as a cofactor. For TMABA-DH, this enzyme has an absolute requirement for NAD+, and its activity is easily measured spectrophotometrically or fluorimetrically by following the appearance of NADH using 4-N-aminotrimethylaminobutyraldehyde as substrate, for example.

For measurement of γBBD, activity is usually measured radiochemically, for example using labelled γBB. The enzyme activity can also be determined by measuring the γBB-dependent release of [¹⁴C]CO₂ that is produced from the decarboxylation of 2-oxo-[1-¹⁴C]glutarate to succinate. This method, however, requires the measurement of γBB-independent activity, since the mitochondrial 2-oxoglutarate dehydrogenase complex also produces CO₂ from 2-oxoglutarate. Alternatively, γBBD activity can be measured using a two-step procedure in which carnitine produced from unlabelled γBB is measured in a radioisotopic assay. In at least certain embodiments for this assay, when tissue homogenates are used, the endogenous carnitine content also needs to be determined. A useful way to measure γBBD is to use stable-isotope labeled γBB and quantify the produced amount of stable-isotope labeled carnitine (van Vlies et al., 2006).

### III. Nucleic Acid Detection

In some embodiments of the invention, at least part of a carnitine metabolic gene, e.g., a TMLHE polynucleotide is detected. The detection may be quantitative, as in providing information on the size of a particular region, and/or the detection may be qualitative, as in provide specific sequence information of a particular region. In certain embodiments, TMLHE probes or primers are utilized for embodiments involving nucleic acid hybridization for detection of mutation(s).

A. Hybridization

In embodiments herein, the invention encompasses one or more probes that hybridizes to nucleotides adjacent to a mutation (including a deletion) of TMLHE, including a specific mutation disclosed herein. The invention also encompasses probes that hybridize wholly outside of a mutation (including a deletion) of TMLHE, including a specific mutation disclosed herein. The invention further encompasses one or more probes that straddle a mutation (including a deletion) site of TMLHE, including a specific mutation discussed herein. Uses of the probes are included in particular aspects of the invention, including in methods to identify one or more particular mutations. Uses of the probes include assaying for a mutation in an individual suspected of having a mutation. The location of a particular mutation in the individual may be unknown, and methods are included for determining or confirming the location of the mutation.

The use of a probe or primer of between 10 and 100 nucleotides, such as between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length may be utilized, for example to increase stability and/or selectivity of the hybrid molecules obtained. One can design nucleic acid molecules for hybridization having one or more complementary sequences of 15 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, it is appreciated that lower stringency conditions may be utilized. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgC12, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR, for detection of TMLHE, including expression therefrom, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

B. Amplification of Nucleic Acids

Nucleic acids used as a template for amplification may be isolated from cells, tissues or other samples according to standard methodologies (Sambrook et al., 1989). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

In embodiments herein, the invention encompasses one or more primers that hybridizes to nucleotides adjacent to a mutation (including a deletion) of TMLHE, including a specific mutation disclosed herein. The invention also encompasses primers that hybridize wholly outside of a mutation (including a deletion) of TMLHE, including a specific mutation disclosed herein. The invention further encompasses one or more primers that straddle a mutation (including a deletion) site of TMLHE, including a specific mutation discussed herein. Uses of the primers include in polymerase chain reaction, for example. Uses of the primers are included in particular aspects of the invention, including in methods to identify one or more particular mutations. Uses of the primers include assaying for a mutation in an individual suspected of having a mutation. The location of a particular mutation in the individual may be unknown, and methods are included for determining or confirming the location of the mutation.

Pairs of primers designed to selectively hybridize to TMLHE nucleic acids are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Affymax technology; Bellus, 1994).

A number of template dependent processes are available to amplify the oligonucleotide sequences present in a given template sample. One of the best known amplification methods is PCR, which is described in detail in U.S. Pat. Nos. 4,683,195, 4,683,202 and 4,800,159, and in Innis et al., 1988, each of which is incorporated herein by reference in their entirety.

A reverse transcriptase PCR amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864, for example.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR and oligonucleotide ligase assy (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

C. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Other Assays

Other methods for genetic screening may be used within the scope of the present invention, for example, to detect mutations in genomic DNA, cDNA and/or RNA samples. Methods used to detect point mutations include denaturing gradient gel electrophoresis (“DGGE”), restriction fragment length polymorphism analysis (“RFLP”), chemical or enzymatic cleavage methods, direct sequencing of target regions amplified by PCR (see above), single-strand conformation polymorphism analysis (“SSCP”) and other methods well known in the art.

One method of screening for point mutations is based on RNase cleavage of base pair mismatches in RNA/DNA or RNA/RNA heteroduplexes. As used herein, the term “mismatch” is defined as a region of one or more unpaired or mispaired nucleotides in a double-stranded RNA/RNA, RNA/DNA or DNA/DNA molecule. This definition thus includes mismatches due to insertion/deletion mutations, as well as single or multiple base point mutations.

U.S. Pat. No. 4,946,773 describes an RNase A mismatch cleavage assay that involves annealing single-stranded DNA or RNA test samples to an RNA probe, and subsequent treatment of the nucleic acid duplexes with RNase A. For the detection of mismatches, the single-stranded products of the RNase A treatment, electrophoretically separated according to size, are compared to similarly treated control duplexes. Samples containing smaller fragments (cleavage products) not seen in the control duplex are scored as positive.

Other investigators have described the use of RNase I in mismatch assays. The use of RNase I for mismatch detection is described in literature from Promega Biotech. Promega markets a kit containing RNase I that is reported to cleave three out of four known mismatches. Others have described using the MutS protein or other DNA-repair enzymes for detection of single-base mismatches.

Alternative methods for detection of deletion, insertion or substitution mutations that may be used in the practice of the present invention are disclosed in U.S. Pat. Nos. 5,849,483, 5,851,770, 5,866,337, 5,925,525 and 5,928,870, each of which is incorporated herein by reference in its entirety.

### IV. Pharmaceutical Preparations

In some embodiments of the invention, an individual having carnitine deficiency and/or one of its precursors and/or defects in TMLHE are provided one or more of carnitine, acetylcarnitine, propionylcarnitine, γBB, or a combination thereof. Such one or more compounds may be formulated in a pharmaceutical composition Pharmaceutical compositions of the present invention comprise an effective amount of one or more of carnitine, acetylcarnitine, propionylcarnitine, γBB or combination thereof dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one of carnitine, acetylcarnitine, or butyrobetaine will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The composition may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The composition may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include carnitine, acetylcarnitine, propionylcarnitine, γBB, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the composition may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to a patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In some embodiments of the invention, a dose of carnitine, acetylcarnitine, propionylcarnitine, γBB, or a combination thereof is delivered to the individual, for example orally. The dosage may be determined by routine methods in the art. In specific cases, however, the dosage is between 1 mg/kg/day and 1000/mg/kg/day, for example, although the dosage may be between 10 mg/kg/day and 500 mg/kg/day, or between 50 mg/kg/day and 250 mg/kg/day, or between 75 mg/kg/day and 200 mg/kg/day, or between 75 mg/kg/day and 150 mg/kg/day, or between 80 mg/kg/day and 125 mg/kg/day, or between 90 mg/kg/day and 115 mg/kg/day. In certain aspects, the dosage is at least 1 mg/kg/day, at least 10 mg/kg/day, at least 25 mg/kg/day, t least 50 mg/kg/day, at least 60 mg/kg/day, at least 70 mg/kg/day, at least 75 mg/kg/day, at least 80 mg/kg/day, at least 90 mg/kg/day, or at least 100 mg/kg/day.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, pharmaceutical compositions may comprise, for example, at least about 0.1% of an active compound. In other embodiments, an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

E. Alimentary Compositions and Formulations

In preferred embodiments of the present invention, the compositions are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

F. Parenteral Compositions and Formulations

In further embodiments, compositions may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

G. Miscellaneous Pharmaceutical Compositions and Formulations

In other preferred embodiments of the invention, the active compound may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### V. Kits of the Invention

Any of the compositions described herein may be comprised in a kit. In a non-limiting example, one or more compounds for determining the level of carnitine or a metabolite of carnitine biosynthesis is provided in a kit in a suitable container means. Non-limiting examples of such compounds include labeled standards of carnitine biosynthesis metabolites, including one or more labeled standards of carnitine, TML, HTML, TMABA, or γBB. The label may comprise an isotope, in certain embodiments.

In another example, one or more compounds for determining whether or not there is a mutation are provided in a kit in a suitable container means. In specific embodiments, the compounds include oligonucleotides, buffers, salts, and so forth. In particular cases, the oligonucleotides are PCR primers for amplifying a region of TMLHE for determination of the presence of one or more mutations in TMLHE. In specific embodiments the PCR primers are capable of amplifying TMLHE sequence to determine if there is a deletion in exon 2, including deletion of the entire exon, for example. In specific cases, the PCR primers target sites outside of exon 2. The PCR primers may target the chromosomal DNA copy of TMLHE or an RT-PCR product of a TMLHE mRNA. Reagents suitable for PCR may be included in the kit, including one or more buffers, salts, deoxynucletoides, and so forth.

In certain embodiments of the invention, there is a substrate having disposed thereon a probe that can specifically detect and distinguish an exon 2-deleted genomic or cDNA molecule, optionally provided with an exon 2 deletion and, optionally, a wildtype control nucleic acid.

All the essential materials and/or reagents required for detecting TMLHE in a sample may be assembled together in a kit. This generally will comprise a probe or primers designed to hybridize specifically to individual nucleic acids of interest in the practice of the present invention. Also included may be enzymes suitable for amplifying nucleic acids, including various polymerases (reverse transcriptase, Taq, etc.), deoxynucleotides and buffers to provide the necessary reaction mixture for amplification. Such kits may also include enzymes and other reagents suitable for detection of specific nucleic acids or amplification products. Such kits generally may comprise, in suitable means, distinct containers for each individual reagent or enzyme as well as for each probe or primer pair.

In some cases, a kit is provided that comprises one or more compounds for determining the level of carnitine or another metabolite in carnitine biosynthesis and the same kit also provides one or more reagents for detecting a mutation in TMLHE.

In some aspects, a compound suitable to raise carnitine levels to an effective amount in an individual are provided in suitable container means in the kit. Exemplary compounds for effectively raising carnitine levels in an individual include carnitine, acetylcarnitine, γBB, or a combination thereof. In some aspects the formulation in the kit is provided for suitable delivery to the individual by oral means.

The components of the kits may be packaged either in aqueous media or in lyophilized form, for example. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the respective compounds and any other reagent containers in close confinement for commercial sale. Such containers may include injection or blow molded plastic containers into which the desired vials are retained.

When the components of the kit are provided in one and/or more liquid solutions, the liquid solution may be an aqueous solution, such as a sterile aqueous solution. The composition may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. In some embodiments, the components of the kit may be provided as dried powder(s). When reagents and/or components are provided as a dry powder, the powder can be reconstituted by the addition of a suitable solvent. It is envisioned that the solvent may also be provided in another container means.

### VI. Array

Embodiments of the invention include a 1 million (1M) Agilent CGH whole-genome exon-focused array with probes selected to give six probes per exon for the majority of exons in the genome. For each exon, three oligonucleotides were selected and both strands were utilized with identical coordinates. Certain genes were omitted on the assumption that they were unlikely to be mutated as a cause of neurobehavioral abnormalities. From the 197,332 unique exons targeted, 98% had <1 probes per exon (PPE) for the exon array. The mean number of probes per exon (PPE) was 5.91 for the custom exon array. Array design Array targets included 273,832 exons from 18,579 genes in the RefSeq database (June 2008, hg18).

In particular, in order to conserve space on the array, 642 genes were removed on the basis that the genes were unlikely to be implicated in a neurodevelopmental disease; these included HLA genes, immunoglobulin genes, T-cell receptor genes, olfactory receptors, and collagen genes. After intentionally removing these genes, 197,332 exons that were unique and mapped to annotated regions remained. All 3′- and 5′-UTRs were included in the list of exons. Six probes were selected to locate partially or completely within the exon. If less than three pairs of probes were not available, the target area was extended into the adjacent intron; three of these probes were on the positive strand and three on the negative strand resulting in three pairs of identical coordinates. The three probe pairs were selected to ensure they were distributed as evenly as possible across each target.

Probes were selected from the Agilent Technologies e-array high density (HD) CGH database. To avoid cross-hybridization each probe was aligned to the hg18 genome using BLAST; any probe that did not map uniquely was removed except for those in the pseudoautosomal regions on chromosomes X and Y for which two locations were tolerated.

Although no probes were used if there was a perfect match elsewhere in the genome, some probes were near-perfect matches and would not distinguish pseudogenes and the parent genes, particularly for unprocessed pseudogenes. Where multiple suitable probes were available, the probes with the highest Agilent HD CGH database probe score where chosen. If the exon length differed between isoforms, the longest possible exon from all isoforms was used to design the six probes.

Sixteen percent of probes were entirely within exons, 56% crossed an intron-exon boundary, 26% were intronic, and 2% were intergenic; 3.5% of probes overlapped a second probe on the same strand often in the case of alternative splicing and overlapping exons. An excess of candidate probes were printed on five Agilent 244K arrays for empirical selection based on performance (i.e., noisy probes were eliminated), and 960,000 probes were chosen to print an Agilent 1M array that was used to generate the data.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Deletion of TMLHE Exon 2 is Recurrent and is Common in Autistic and Healthy Males

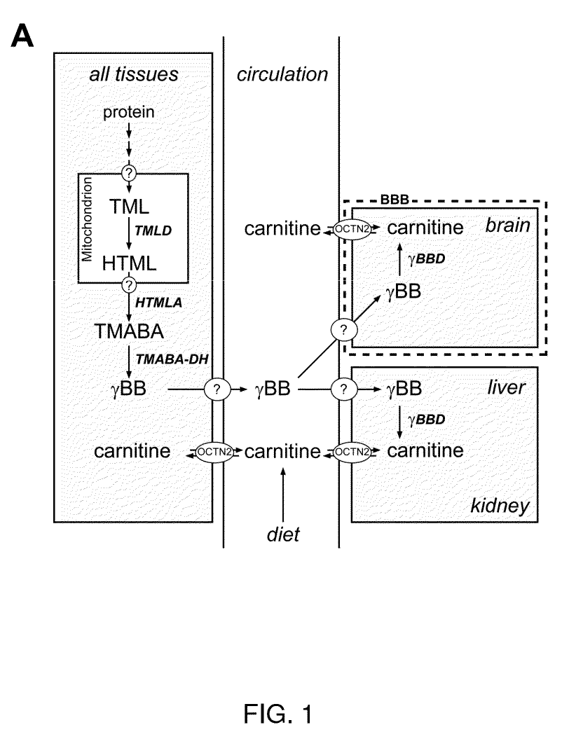

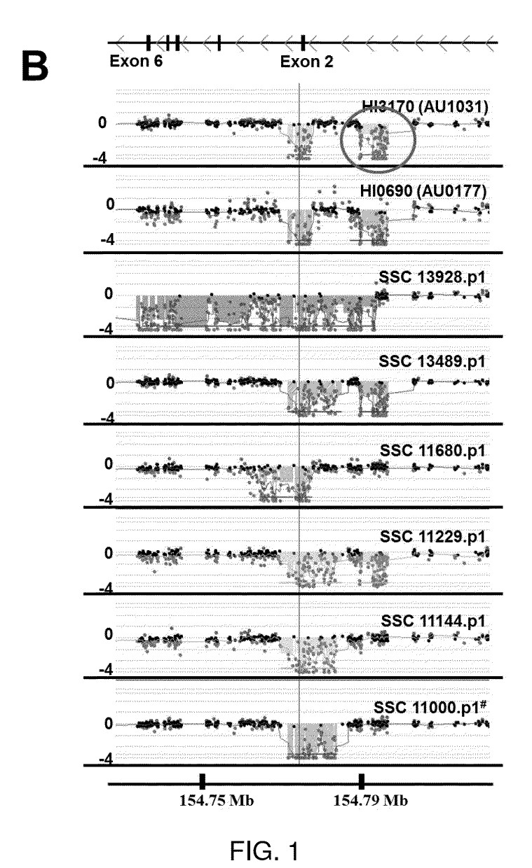

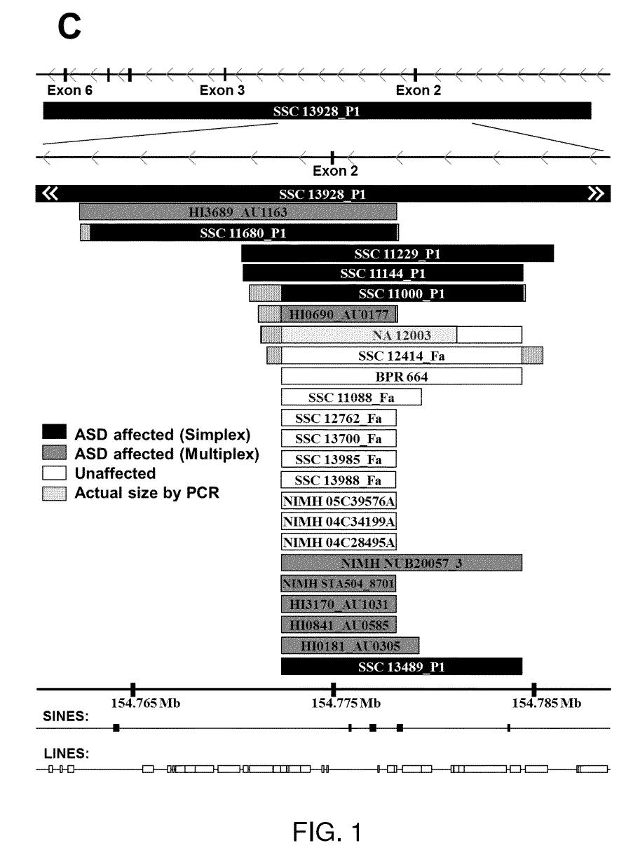

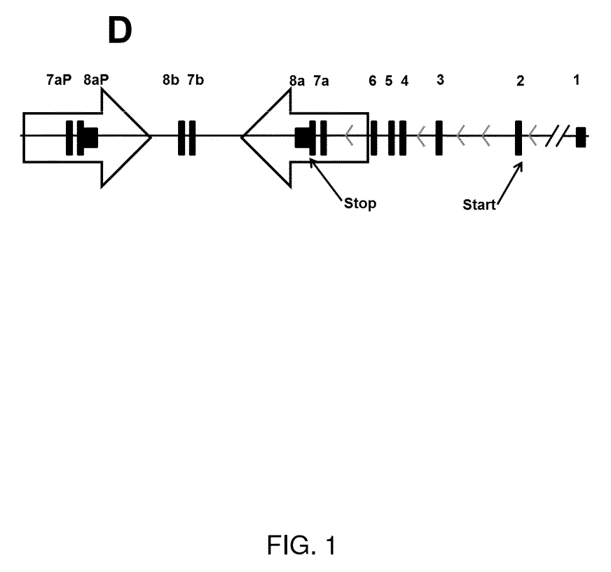

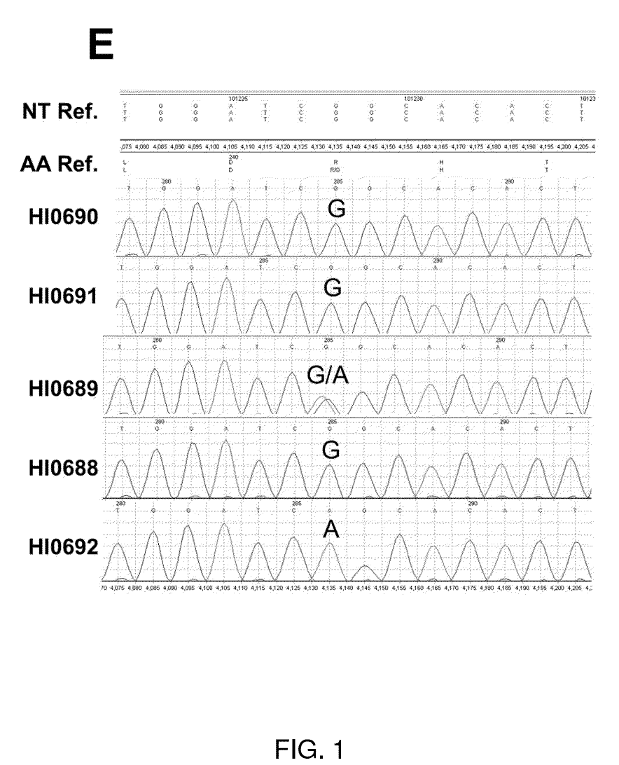

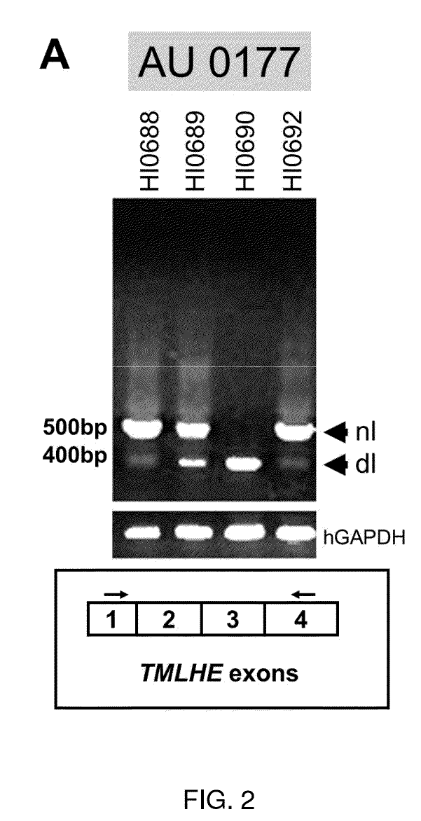

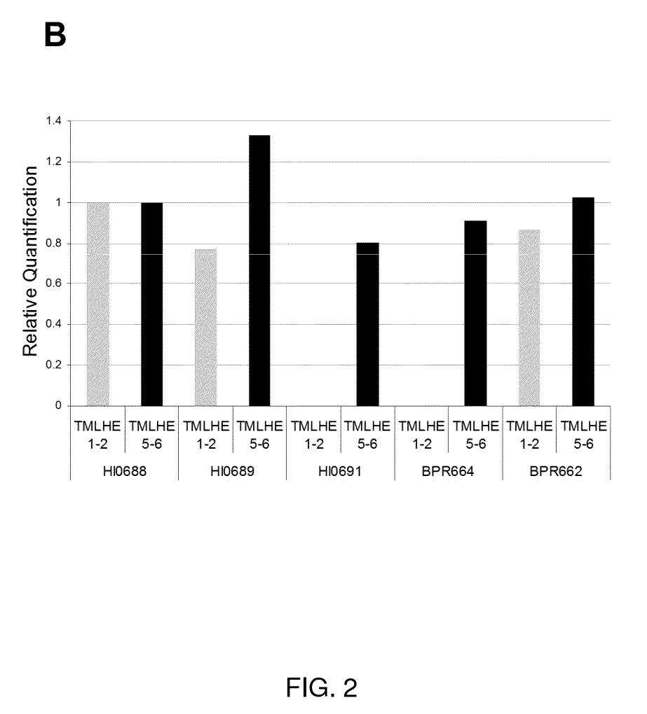

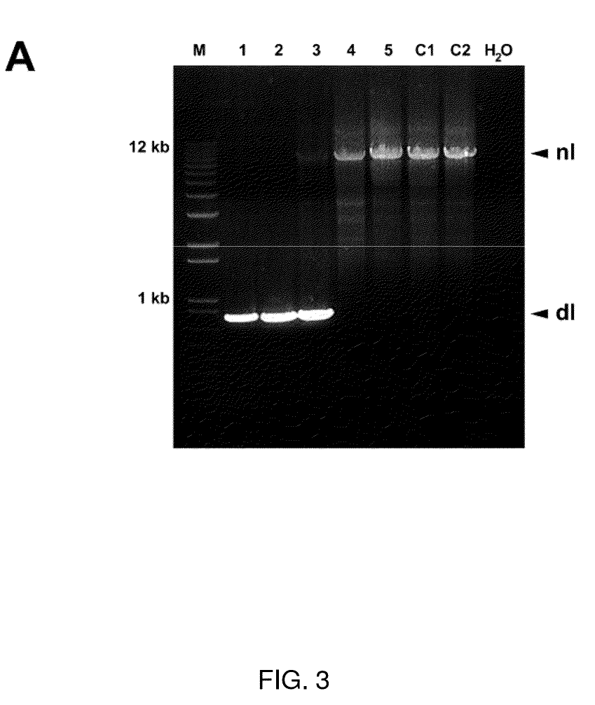

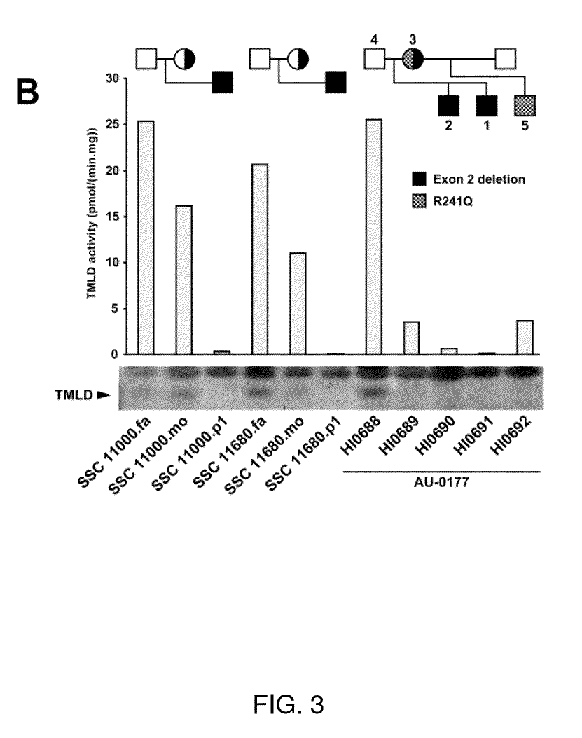

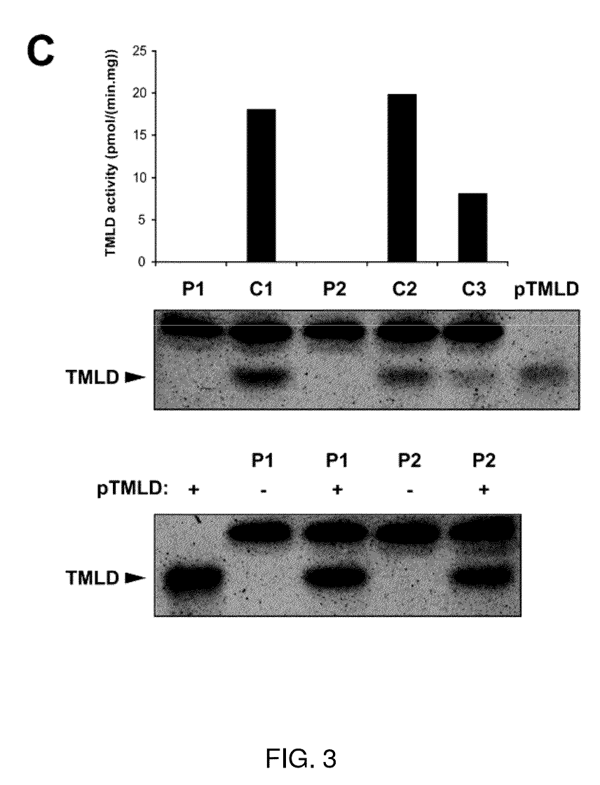

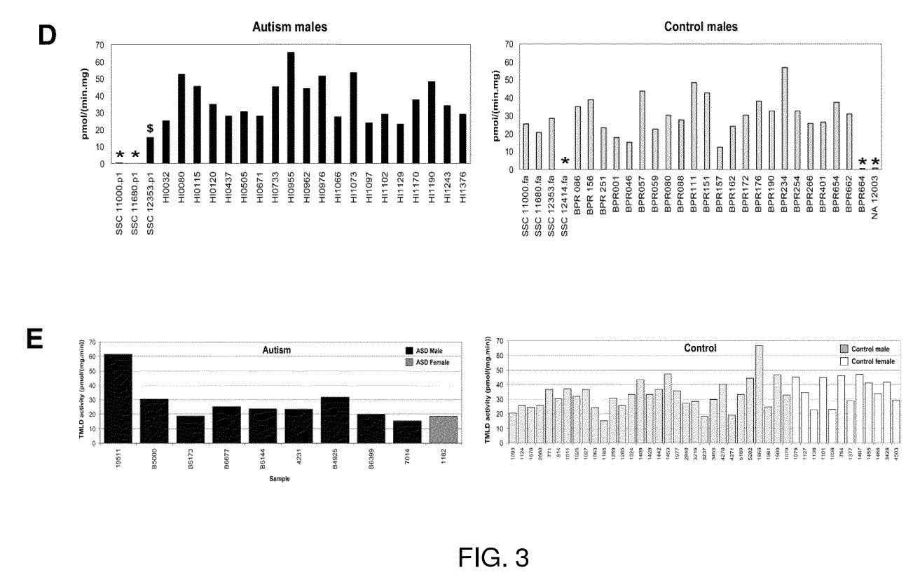

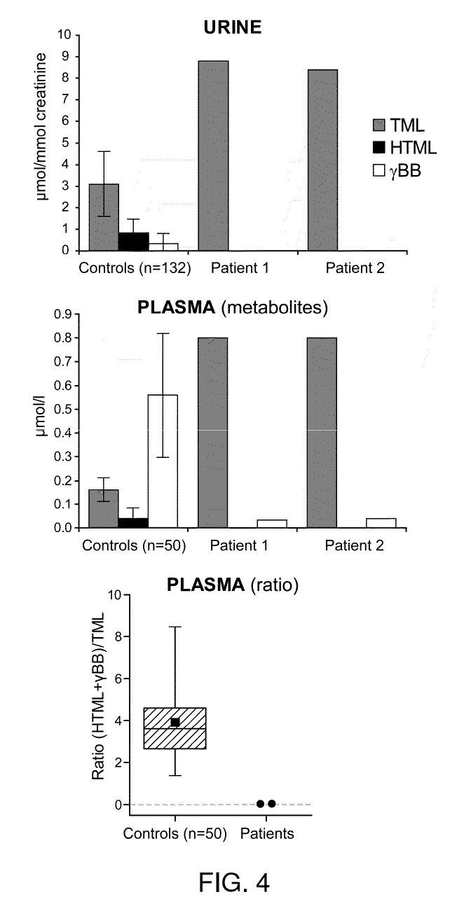

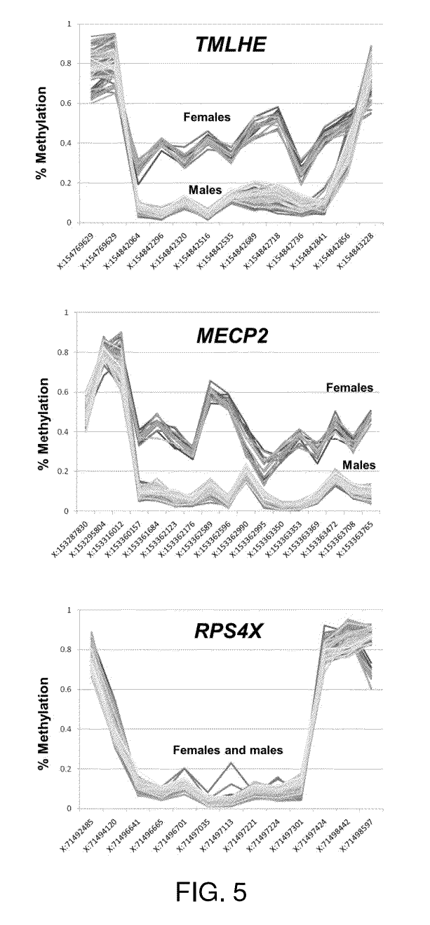

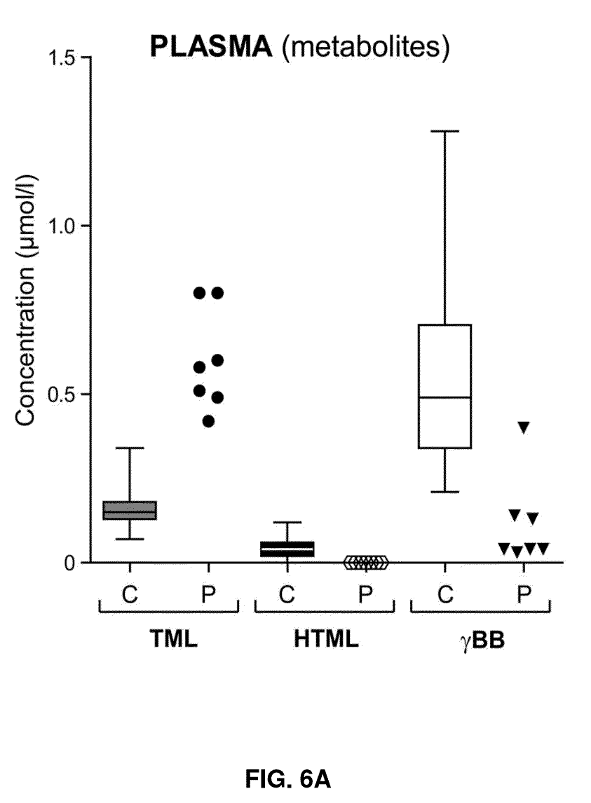

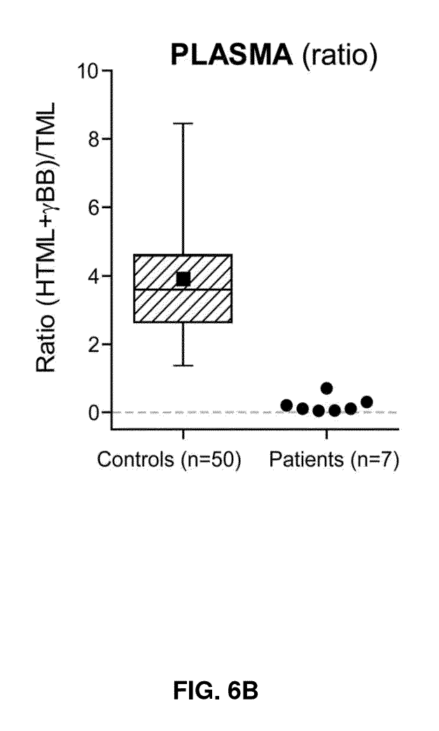

In addition to the first family found to have deletion of exon 2 of TMLHE, there were many other autism probands and healthy adult males with deletions of exon 2 indicating that this is a relatively common CNV. There is at least one family with the deletion in two brothers with autism from the AGRE collection (family AU 0177). Deletions of exon 2 ranged in size from 5.7 to 15.9 kb except for one deletion of 59.6 kb which deleted exons 2-6 (FIGS. 1B and 1C and Table S1 (see below)). Based on position and size, 14 of 24 deletions were different from all others; there were 10 deletions that were very similar in size. Sequencing of the breakpoints of many deletions showed that almost all junctions occurred in LINEs and S1NEs in the introns flanking exon 2 (Table S2 (see below)) as has been seen in other loci (Boone et al., 2011). These results indicate recurrent deletions with slightly different junctions.

In addition, there was a very common intronic deletion (FIG. 1B) that appears on the basis of array CGH to be identical in all cases. The intronic deletion is extremely common and was present in 74% of 93 autism male probands tested and 71% of 48 control males. This intronic deletion was present in cis on 20/24 mutant chromosomes from unrelated families with exon 2 deletion of TMLHE (Table S1 (see below)).

Genomic sequencing of exons for TMLHE is complicated by the presence of two pseudoexons (7aP and 8aP) that are highly homologous to exons 7a and 8a and imbedded in a large inverted repeat downstream of TMLHE (FIG. 1D). In addition, there are two alternative exons 7b and 8b with sequences unrelated to 7a and 8a which are located between the two inverted repeats (Monfregola et al., 2007). Sequencing of exons 1 to 8 of TMLHE from genomic DNA in 536 SSC autism male probands and 98 AGRE probands from multiplex families with at least two affected brothers identified a glutamic acid to lysine substitution in codon 287 (E287K) in an SSC proband (SSC 12353.p1). The mutation was inherited from the heterozygous mother and also present in a healthy sister. Sequencing in 443 NIMH male controls identified a valine to isoleucine substitution in codon 74 (V74I) (NIMH 04C26633A). In addition to exon 2 deletion, sequencing in the AU 0177 family identified an arginine to glutamine change in codon 241 (R241Q) in the mother and unaffected half brother (FIG. 1E). Sequences of exons 7a and 8a were judged to be unreliable, because they could not be amplified uniquely free of product from pseudoexons 7aP and 8aP; however, we identified a heterozygous-like valine to isoleucine substitution in codon 349 (V349I in exon 7a/7aP) in a SSC male proband (12041.p1). Table S3 contains a summary of the mutations found.

Analysis of RNA from lymphoblast cell lines using RT-PCR allows for sequencing of the transcript of exons 7 and 8 without interference from the pseudoexons. This analysis also revealed low levels of skipping of exon 2 in most samples and a stable transcript with complete absence of exon 2 in cells from males with deletion of this exon (FIG. 2). Thus nonsense mediated decay does not occur for the exon 2 deletion transcript. This was confirmed using qRT-PCR which showed normal levels of transcript for the exon 5-6 junction in all samples but complete absence of the exon 1-2 junction in deletion samples.

### Example 2

### Exon 2 Deletion Results in Loss of TMLD Activity, Absence of TMLD Protein, and Diagnostic Metabolite Abnormalities

Cultured lymphoblast lines from the numerous exon 2 deletion samples showed low or undetectable TMLD enzyme activity. Results from family AU 0177 were complex with the two affected brothers with deletion of exon 2 having very low or undetectable enzyme activity, but the unaffected mother and half brother showing low activity but higher than the affected brothers (FIGS. 3A, 3B, and Table S4). The mother is a compound heterozygote with deletion of exon 2 on one X chromosome and the R241Q mutation on the other; she transmitted the deletion to her two affected sons and the R241Q mutation to their unaffected half-brother. Western blot analysis did not reveal any immunoreactive material in deletion lymphoblast lines in contrast to detection of the expected band in control cell lines (FIGS. 3B and 3C). Enzyme activity results for multiple autism cell lines and controls demonstrated that deletion of exon 2 resulted in undetectable enzyme activity (FIG. 3D); studies included a total of 4 autism cell lines (SSC 11000.p1, SSC 11680.p1, HI0690_AU 0177, HI0691_AU 0177) and 5 control cell lines (SSC 12414.fa, BPR664, NA12003, HI0689_AU 0177, HI0692_AU 0177).

Plasma and urine from the two affected brothers in AGRE family AU 0177 were analyzed for changes in metabolite concentrations (FIGS. 4 and 6). They both had normal plasma carnitine levels but showed a significant increase in TML, complete absence of HTML, and severely reduced levels of γBB. In urine of the affected brothers, HTML and γBB were undetectable, and the excretion of TML was 3-fold that of controls. The (HTML+γBB)/TML ratio was very low in patients as compared to control plasma and may be an excellent index of TMLD activity (FIGS. 4 and 6). These data indicate that TMLHE deficiency represents a novel inborn error of carnitine biosynthesis. However, at least at this point any link to autism was still uncertain, because many control males showed deletion of exon 2.

To further characterize the embodiment that carnitine metabolism plays a role in NDA, TMLD activity was measured in a series of autism and control cerebellum samples (FIG. 3E and Table S5). Cerebellum was established as a brain region of interest long ago because of evidence of Purkinje cell dropout in autism (Bauman and Kemper, 2005); fortuitously, TMLHE is highly expressed in Purkinje cells, at least in mice (Monfregola et al., 2007). There was no evidence for brain-specific deficiency of TMLD activity in cerebellum. This argued against the possibility of a common mutation or epimutation involving TMLHE as a risk factor for autism. In addition, activity was not significantly higher in control female versus control male cerebellum as might occur if there was incomplete X-inactivation of TMLHE. Carnitine metabolites in autism and control brain were also measured (Table 1 and Table S5).

TABLE 1
Carnitine Biosynthesis Metabolites in Human Cerebellum

| | Male control | Female control | All controls | Autism |
|---|---|---|---|---|
| | (n = 33) | (n = 12) | (n = 45) | (n = 9) |
| | pmol/mg protein, mean ± SD | | | |
| TML | 263 ± 206 | 251 ± 147 | 260 ± 189 | 191 ± 77 |
| HTML | 15 ± 11 | 12 ± 3 | 14 ± 10 | 12 ± 5 |
| γBB | 33 ± 11 | 39 ± 25 | 34 ± 16 | 46 ± 16* |
| Carnitine | 861 ± 296 | 702 ± 166 | 816 ± 274 | 597 ± 294# |

*p < 0.01 autism compared to male controls
#p < 0.05 autism compared to male controls and to all controls
See also Table S5

TML and HTML levels were not different between groups. Carnitine levels, however, were significantly lower in autism when compared to controls (p=0.04, unpaired t-test, 2 tailed) and even more significant when compared to male controls (p=0.02). Female controls showed a trend towards lower carnitine levels when compared to male controls levels, but this was not significant (p=0.09). In contrast to carnitine, significantly higher γBB levels were found in autism brain. However, measurement of γ-butyrobetaine dioxygenase (γBBD) activity in cerebellum did not show any difference in autism compared to controls that might explain altered γBB/carnitine ratios.

### Example 3

TMLHE Exon 2 Deletion is Significantly More Frequent in Multiplex Autism

It was important to determine whether there was a significant association of TMLHE deficiency with autism. Because deletion of exon 2 was more common than any other mutations detectable by genomic sequencing, and because it was associated with loss of enzyme activity, it was expedient to analyze a large series of autism cases and controls for exon 2 deletion. A PCR assay was designed with primers slightly outside the boundaries of exon 2 to give a product of ˜500 bp in normal males but no product for males with deletion of exon 2. If a sample failed to give a PCR product, the presence or absence of deletion was then confirmed using custom array CGH with densely spaced oligonucleotides interrogating the TMLHE region (FIG. 1B). For PCR analysis, we focused entirely on males, because the assay would not reliably detect the deletion in heterozygous females.

Using the PCR assay, the inventors tested 1803 SSC simplex male probands, 2101 SSC unaffected fathers, many multiplex probands (multiplex here refers to male-male sib pairs both affected with autism) and unaffected fathers from multiplex families from the AGRE and other collections, and NIMH “control” males as summarized in Table 2. For each multiplex family, only one affected male was tested initially.

TABLE 2
Frequency of Exon 2 Deletion in Male Autism Cases and Controls

| Samples | Probands | Controls | P value^{a} |
|---|---|---|---|
| SSC males | 6/1803 | 6/2101fathers | |
| Multiplex AGRE male proband | 5/494 | 0/402 fathers | |
| pairs | | | |
| Multiplex other male proband pairs | 2/258 | 0/213 fathers | |
| NIMH “controls” | None | 3/897 | |
| SSC probands vs. all controls | 6/1803 | 9/3612 | 0.38 |
| | 0.33% | 0.25% | |
| Multiplex probands vs. all controls | 7/752 | 9/3612 | 0.012* |
| | 0.93% | 0.25% | |
| Multiplex probands vs. multiplex | 7/752 | 0/615 | 0.015* |
| fathers | 0.93% | 0 | |

^{a}One-tail Fisher's exact test
*Significant difference
See also Table S6

Deletion of exon 2 was 1.3 times more common in SSC probands compared to all control males, but this difference was not statistically significant (p=0.38). The difference between SSC probands and SSC fathers was insignificant. Deletion of exon 2 was 3.7 times more common in multiplex probands of male-male affected sib pairs with autism compared to all control males, resulting in a statistically significant difference (p=0.012). When comparing multiplex probands to unaffected fathers from the same multiplex families to minimize stratification, the result was also significant with p=0.016. The data from multiplex families suggested that there likely was a true association of TMLHE deficiency with autism, but that this association was relatively weak primarily related to lack of penetrance in healthy males. The NIMH control collection is known to have higher prevalence than other series for depressive, anxiety, and substance use diagnoses (Sanders et al., 2010); if these controls were excluded, the p-value comparing multiplex probands to autism fathers would be 0.011. Nonetheless, approximately 1 in 400 apparently unaffected males were deleted for exon 2 of TMLHE. All genotypes were consistent with the X-linked inheritance; all mothers of probands with deletion of exon 2 who were tested were heterozygous for the deletion, and 6 of 7 affected siblings of the multiplex male pairs had the deletion, which is consistent with the conclusion that there is a significant association of TMLHE deficiency with autism.

Significant phenotypic information is available for the 6 SSC probands, 6 SSC unaffected fathers, 7 multiplex and 7 affected brothers, and 3 NIMH control males with deletion of exon 2 of TMLHE (Table S6). The levels of cognitive and language functioning varied considerably across cases. A history of regression was rare. One SSC proband (11680.p1) also had a 16p11.2 deletion. With respect to the controls, two of the six SSC fathers had at least one domain with an elevated broader autism phenotype score based on self-report (BAP-Q), but the Social Responsiveness Scale (SRS-ARV) rating by significant other and Family History Interview-Interviewee Impression (FHI-I) scores were not consistent with broader autism phenotype.

### Example 4

The Sex Ratio in NDA is not Caused by a Common Mutation, Epimutation, or Incomplete X-Inactivation of TMLHE

Using publicly available SNP genotyping data obtained from AGRE on the AGRE collection of families, we performed linkage analysis for TMLHE and for the nearby VAMP7 gene which is in the Xq pseudoautosomal region and is potentially relevant to synaptic function. For both gene regions, no significant evidence of linkage was observed. For the TMLHE region a maximum nonparametric linkage (NPL)-score of 1.25 and LOD score of 0.34 was observed for markers located within and flanking the TMLHE gene. For the VAMP7 gene region a maximum NPL score of 0.76 and LOD score of 0.15 was observed.

Because the TMLHE gene is on the X-chromosome, and because the sex ratio in mild autism can be as high as 8:1 male:female (Scott et al., 2002; Kalra et al., 2005), it was important to probe the hypothesis that X-linked dysfunction of TMLHE might somehow explain the extreme sex ratio in NDA. We considered the possibilities of a common but difficult to detect mutation, epimutations, or incomplete X-inactivation. The linkage data argue against the first of these. We examined DNA methylation in autism and control cerebellum to assess the other two possibilities. It was previously reported that TMLHE was subject to tight X-inactivation in cultured human cell lines (Carrel and Willard, 2005). There were differences of DNA methylation between male and female control and autism cerebellum samples that were consistent with what might be expected for a gene subject to X-inactivation (FIG. 5) with methylation very low in male brain and intermediate in female brain, presumably reflecting one methylated inactive X and an unmethylated active X. This is compared to a typical gene subject to X inactivation (MECP2 in FIG. 5), and to a typical gene not subject to X-inactivation (RPS4X in FIG. 5). Analysis of DNA methylation using autism and control cerebellum did not suggest any gain of methylation that might be associated with silencing epimutations of TMLHE in male cerebellum samples (FIG. 5). Analysis of TMLD activity in autism lymphoblast lines (FIG. 3D) and cerebellum (FIG. 3E) argues against a common mutation or epimutation and against incomplete X-inactivation, since TMLD activity was not significantly higher in female brain compared to male brain.

### Example 5

### Mutations in the SLC22A5 Carnitine Transporter Gene are Frequent in Simplex Regressive Autism but Association is not Established

The evidence that TMLHE deficiency was associated with autism made numerous carnitine-related enzymes and transporters candidates for study in autism. The best characterized gene with human disease involvement is the SLC22A5 gene that encodes the OCTN2 high affinity carnitine transporter. Homozygous deficiency for this gene causes systemic carnitine deficiency, and heterozygous deficiency is associated with decreased plasma carnitine in humans and mice (Koizumi et al., 1999; Lahjouji et al., 2002). In certain embodiments of the invention, heterozygous deficiency for SLC22A5 is associated with autism and episodes of reduced carnitine availability leads to regression. Therefore, the inventors performed mutation analysis and copy number analysis for SLC22A5 on 277 families from the SSC selected for the presence of a history of regression in the proband and for the availability of a healthy sibling. There were 7 known deficiency mutations in parents where the mutation was transmitted to probands and 4 such mutations in parents where the mutation was not transmitted to the proband (Tables S7 and S8). The presence of 11 deficiency mutations in 277 pairs of parents indicates a carrier frequency of 1 in 50. A carrier frequency of 1 in 100 was found in the Japanese (Koizumi et al., 1999). The frequency of transmitted deficiency mutations was 1.75 times higher than for non-transmitted mutations; the trend is consistent with the heterozygous deficiency for SLC22A5 being associated with regressive autism. The inventors next sequenced SLC22A5 exons in 92 multiplex probands from male-male autism sib pairs from multiplex AGRE families not selected for regression, and no known deficiency mutations were found. As described for TMLHE and VAMP7 above, linkage analysis was performed for SLC22A5 yielding a maximum NLP-score and LOD score of 0.0 across the region. Much larger numbers of simplex and multiplex autism families with and without regression should be studied to resolve whether there is any association of SLC22A5 genotype with autism.

### Example 6

### TMLHE Deficiency, a New Inborn Error of Metabolism

TMLHE deficiency is a new inborn error of metabolism, discovered about one hundred years after Garrod first described such conditions. The frequency of the deficiency is startling at approximately 1 in 400 males, making it at least 20 times more frequent than phenylketonuria in males. However, the majority of affected males are phenotypically “normal” as adults. Assuming 4 million births per year in the USA, this would equate to about 5,000 deficient males born per year. If the association with autism is valid and the penetrance in the U.S.A. is 2-4% as we believe is a reasonable estimate, this would mean 100 to 200 cases of autism associated with TMLHE deficiency occurring per year. As much information as possible was gathered regarding the phenotype for 18 males with autism and for 7 unaffected males having TMLHE deficiency as summarized in Table S6. Regression was not a frequent finding. In certain aspects of the invention, other behavioral phenotypes common in males (e.g., ADHD and Tourette syndromes and broad autism phenotype) are associated with THMLE deficiency.

Biochemically, TMLHE deficiency is characterized by deficient enzyme activity and by increased substrate (TML) and decreased products (HTML and γBB) in plasma and urine. In all cases examined, the exon 2 deletion results in complete loss of protein expression, but point mutations also were detected that showed residual activity (FIG. 3D). TMLD is the first enzyme of carnitine biosynthesis but the role of this pathway in carnitine homeostasis remains enigmatic. As shown in FIG. 1A, not all tissues are capable of complete carnitine biosynthesis due to the differential expression of the last enzyme, γBBD, which in humans is only expressed in kidney, liver and brain. After degradation of proteins that contain TML residues, TML is converted to γBB which is then transported to the tissues that express γBBD and converted into carnitine. The plasma membrane γBB transporter likely is encoded by the SLC6A13 gene, which is known as a betaine/GABA transporter and has recently been suggested to function in carnitine biosynthesis as the liver γBB transporter (Fujita et al., 2009). The structural resemblance of γBB (4-N-trimethylaminobutyric acid) to GABA (4-aminobutyric acid) and betaine (2-N-trimethylamino ethanoic acid) and the fact that GABA transporters can handle γBB as substrate might point to a function of γBB other than in carnitine biosynthesis, possibly in neurotransmission. Dysfunction of GABAergic neurotransmission has been implicated in mood disorders (Brambilla et al., 2003), and it recently has been suggested that subtle dysfunction of GABAergic neurons could contribute to numerous neuropsychiatric phenotypes, including autism (Chao et al., 2010). In contrast to carnitine levels, γBB concentrations were severely reduced in TMLD deficient individuals and in at least certain aspects of the invention the deficiency of γBB plays a role in the etiology of autism by influencing GABAergic neurotransmission.

### A Link to Autism

As noted in the Introduction, TMLHE is strongly expressed in Purkinje cells and hippocampal neurons in the mouse (Monfregola et al., 2007), which is of interest as there is evidence of Purkinje cell dropout in autism (Bauman and Kemper, 2005). Given that there is an association in male-male multiplex families, but not in simplex families, this is consistent with the presence of a true association. In embodiments of the association, it is more evident in multiplex male families than in simplex families. The presence of the deletion in 6 of 7 affected brothers of the multiplex probands also indicates that there is a true association. The findings that deletions of exon 2 are recurrent and molecularly heterogeneous, while the deletion in intron 1 may be homogeneous, are compatible with the deletions of exon 2 being selected against in recent generations, while the intronic deletion may not be subject to negative selection. The frequency of deletions of exon 2 may vary among racial or ethnic groups, perhaps depending on polymorphisms in the LINEs and SINEs in introns 1 and 2 of TMLHE in different ethnic groups, and other mutations may be more common in such groups.

In embodiments of the invention, there is an association with simplex autism. Here the inventors considered the use of unaffected fathers of probands as controls. This has the advantage of matching generally for ethnicity, but the X chromosomes of the fathers and sons are not shared. Samples such those of the Wellcome Trust Case Control Consortium (Craddock et al., 2010) may be examined and factors including ancestry of the population and dietary practices in infancy may be considered. If penetrance is influenced by carnitine intake during infancy, it may be greater in countries with a high frequency of vegetarian diets and lower meat or beef intake. China, India, and South Korea are all countries where some studies of incidence of autism are available (Kalra et al., 2005; Wong and Hui, 2008; Kim et al., 2011), and there is a more vegetarian diet and/or a much lower beef intake.

### Preventive and Therapeutic Implications

In embodiments of the invention autism in children with TMLHE deficiency can be reversed in very young children or prevented altogether. Carnitine supplementation is useful in children identifiable with a diagnosis of autism and TMLHE deficiency. In embodiments of the invention, carnitine and/or γBB supplementation is beneficial, although toxicity of TML, deficiency of other carnitine biosynthesis intermediates, or some unknown moonlighting function of the TMLD enzyme is considered. In certain aspects of the invention, the use of L-carnitine, acetyl- or propionyl-carnitine, and/or γBB is compared. There is a recent report of a trial of carnitine supplementation in autism (Geier et al., 2011), yet it would be desirable to have data from much younger patients, preferably nondysmorphic, with and without TMLHE deficiency.

### Is Carnitine a (Conditionally) Essential Nutrient?

There has been considerable debate over decades as to whether carnitine is an essential nutrient deserving assignment of minimal daily requirements as noted in the Introduction (Conference Proceedings, 2004). Generally no minimum daily requirements are assigned, and it has been thought that most or all humans can synthesize carnitine, and that some balance of dietary intake and endogenous synthesis meets the requirements for normal health and development. The fact that as many as 1 in 400 males cannot synthesize carnitine may challenge the lack of need for minimum daily requirements.

Symptomatic carnitine deficiency can arise through many genetic, dietary, and drug-induced states. One mechanism is through inborn errors of organic acid and fatty acid metabolism that lead to loss of carnitine metabolites in the urine (Flanagan et al., 2010); another is through primary carnitine deficiency. In embodiments of the invention, a male with TMLHE deficiency would become ill if placed on a very low carnitine diet, and carnitine is an essential nutrient at least for these individuals. There is evidence that total parenteral alimentation in the absence of carnitine supplementation (Worthley et al., 1983; Okanari et al., 2007) and use of soy formula lacking carnitine supplementation can cause symptomatic carnitine deficiency (Slonim et al., 1981), and TMLHE deficient males would be particularly susceptible. From a strictly dietary perspective, cereals, fruits, and vegetables are virtually devoid of carnitine, while milk, eggs, fish, and poultry have intermediate amounts. Beef and other red meats have extremely high carnitine content such that the carnitine content per calorie can vary by three orders of magnitude across these food categories. Thus vegetarian diets are quite low in carnitine content and a vegan diet, defined as excluding all animal products such as eggs and dairy, is especially low even compared to an ovo-vegetarian or lacto-vegetarian diet.

### Other Carnitine Related Genes

Carnitine homeostasis involves at least eight genes, including TMLHE (FIG. 1A). Other than TMLHE deficiency identified in this paper, no other defect of carnitine biosynthesis is known. As TMLD is a mitochondrial enzyme, one or more transport proteins are required to import TML into mitochondria and to export HTML back to the cytosol where the rest of the carnitine biosynthesis enzymes are located. Deficiency of this transport system has the same metabolic consequences as TMLHE deficiency, in particular embodiments of the invention. Mutations in HTMLA, TMABA-DH (encoded by ALDH9A) and γBBD (encoded by BBOX1) lead to defective carnitine biosynthesis, in specific aspects of the invention. As mentioned above, given the similarity of γBB to GABA, its deficiency or accumulation disturbs GABAergic neurotransmission, in certain aspects of the invention, which in turn plays a role in the etiology of autism, in embodiments of the invention. In that respect, the γBB transporter, for example SLC6A13, is of particular interest in methods and compositions of the invention.

The best characterized defect of carnitine homeostasis is systemic primary carnitine deficiency which is caused by biallelic null or hypomorphic mutations in the SLC22A5 gene on chromosome 5 which encodes the organic cation transporter 2 (OCTN2). In alternative embodiments of the invention, heterozygous loss-of-function mutations in SLC22A5 contribute to carnitine deficiency and are a risk factor for autism.

One might ask why autism is not reported more frequently in primary carnitine deficiency. In embodiments of the invention, this is explained by the neuronal carnitine pathway embodiments. In these embodiments, even when plasma carnitine is very low, neurons are able to synthesize carnitine and would have normal concentrations of carnitine biosynthesis intermediates that play an important role in the brain. In specific embodiments, systemic carnitine deficiency and neuronal carnitine or carnitine precursor deficiency have very different pathophysiologies with plasma carnitine levels often being low and neuronal levels being normal in systemic carnitine deficiency as contrasted to TMLHE deficiency where carnitine levels might be normal in plasma but low in neurons/CSF. CSF carnitine is measured in autism cases with and without TMLHE deficiency.

### A Neuronal Carnitine Pathway Hypothesis for Autism

There are particular embodiments of the invention involving neuronal deficiency of carnitine or other downstream metabolites of TML in the etiology of autism. This carnitine pathway embodiment could include harm caused by TML toxicity or by deficiency of HTML, γBB, or carnitine, for example. Carnitine supplementation is expected to correct only carnitine deficiency, while supplementation with γBB may correct either carnitine or γBB deficiency, in specific aspects. These supplements may not protect against toxicity of TML or deficiency of HTML or unknown derivatives thereof. In embodiments wherein the penetrance of TMLHE deficiency is influenced by dietary carnitine, the embodiment of carnitine deficiency per se would be the most attractive. The inventors found significantly lower carnitine levels in a relatively small set of autism cerebellum samples. A carnitine pathway embodiment for autism encompasses neuronal carnitine deficiency primarily on a nutritional basis and/or disturbances of metabolites other than carnitine and could 1) involve other carnitine-related genetic variations, 2) explain regression in autism caused by transient decrease of carnitine availability during development, 3) establish a link to mitochondrial function, 4) indicate further genetic and biochemical studies to test the hypothesis, 5) involve a critical diet-genotype interaction, and/or 6) lead to early diagnosis and treatment for patients in whom carnitine deficiency is the primary cause of their autism.

### Example 7

### Exemplary Experimental Procedures

### Subjects

For SSC, AGRE, and NIMH samples, DNA derived from lymphoblastoid cell lines (LCLs) was obtained from the Rutgers University Cell and DNA Repository. Blood from Nashville, Tenn., individuals was used for establishment of LCLs and DNA was extracted with Puregene chemistry on the Autopure (Qiagen, Valencia, Calif., USA). Blood from adult normal Caucasian BPR (Baylor Polymorphism Resource, Houston, Tex.) individuals was used for the establishment of lymphoblastoid cell lines (LCLs). LCL derived DNA was used for array comparative genomic hybridization (CGH) and PCR, unless otherwise specified.

For PCR analysis of exon 2 of TMLHE, the inventors studied 1803 male probands and 2101 unaffected fathers from the Simons Simplex Collection (SSC). The SSC enrolls young simplex cases of autism as described elsewhere (Fischbach and Lord, 2010). In addition, we studied 494 autism males from the Autism Genetic Resource Exchange (AGRE) (see World Wide Web site) all of whom had an affected brother, 402 unaffected fathers from AGRE, 258 local (Nashville, Tenn.) or National Institute of Mental Health (NIMH) (see World Wide Web site) autism males, all of whom had an affected brother, 213 local (Nashville, Tenn.) or NIMH unaffected fathers, and 897 male controls from the National Institute of Mental Health Human Genetics Initiative (NIMH-HGI) (see World Wide Web site).

PCR and sequencing of TMLHE exons 1-8 was performed for 536 SSC male probands, 98 affected AGRE males from male-male multiplex families (brothers or half-brothers with same mother), and 443 NIMH male controls (see World Wide Web site) (Table S3).

### Array CGH

All arrays used in this study were designed and analyzed based on UCSC hg18 (NCBI Build 36), March 2006. The coordinates found in tables and figures are converted to hg19 (GRCh Build 37), February 2009. An Agilent custom CGH array of design ID 028249 was used to confirm TMLHE deletions originally found by PCR or that were detected by the 1M Illumina SNP array through a collaborative study of SSC families (Sanders et al., 2011). The custom array design is available on the Agilent's eArray website (see World Wide Web site). Analysis of CNVs was done using Agilent's DNA Analytics software (v4.0.76) with the following settings: aberration algorithm ADM-2, minimum of 3 consecutive probes per region, and a minimum absolute average log 2 ratio of 0.25 for any given region.

The protocol for DNA digestion, labelling, purification, and hybridization to the arrays followed the manufacturers' instructions with some modifications, as described previously (Ou et al., 2008). Genomic DNA (800 ng) from the SSC individual and from a single male reference were used in the digestion. Each slide was scanned into an image file using the Agilent G2565 DNA Microarray Scanner at a 3 micron scan resolution. Each image file was quantified using Agilent Feature Extraction software (v10.7.3.1). The Agilent custom focused validation files were uploaded into the Agilent DNA Analytics software (v4.0.76) for analysis.

### Enzyme Assays

All individuals tested for TMLD enzyme activity were assayed for presence or absence of exon 2 of TMLHE by PCR or aCGH. These included BPR controls, AGRE and SSC individuals, NA12003 (McCarroll et al., 2006), and brain samples. TML was obtained from Sigma-Aldrich. [²H₉]TML and [²H₃]γ-butyrobetaine (γBB) were synthesized as described previously (Vaz et al., 2002). [²H₉]HTML was prepared enzymatically by incubating [2H9]TML with Neurospora crassa 6-N-trimethyllysine dioxygenase, heterologously expressed in Saccharomyces cerevisiae as described previously (Swiegers et al., 2002). The resulting mixture of [2H9]HTML and [2H9]TML was applied to Amicon Ultra 30-kDa filters (Millipore, Ireland) and the deproteinized filtrate was used as internal standard for TML and HTML. All other reagents were of analytical grade.

Frozen cerebellum tissues and lymphoblast pellets were homogenized in 10 mM Mops buffer containing 0.9% (w/v) NaCl, 10% (w/v) glycerol, and 5 mM dithiothreitol (DTT), pH 7.4. The protein concentration was determined by the method of Bradford (Bradford, 1976) using human serum albumin (HSA) as standard. For measurement of TMLD activity, the reaction mixture consisted of 20 mM potassium phosphate buffer containing 50 mM KCl, 3 mM 2-oxoglutarate, 10 mM sodium ascorbate, 0.5 mM DTT, 0.5 mM ammonium iron sulfate, 2.5 mg/ml BSA and 2 mM TML, pH 7.4 with a final volume of 250 μl. The reaction was started by adding 50 μl of homogenate (target final protein concentration of 0.2 mg/ml for lymphoblast homogenates and 1 mg/ml for brain homogenates) to the reaction mixture and was incubated 37° C. for 30 minutes. The reaction was terminated by the addition of ZnCl₂ to a final concentration of 1 mM and the reaction mixtures were placed on ice. The ZnC12 solution also contained the internal standards: 50 pmol [²H₉]HTML, 140 pmol [²H₉]TML, 140 pmol [²H₃]γ-BB and 550 pmol [²H₃]carnitine. Subsequently, the reaction mixture was loaded onto an Amicon Ultra 30-kDa filter and centrifuged at 14000×g for 20 min to separate the metabolites (TML, HTML, γ-BB, and carnitine) from the enzymes and remove most of the proteins. 100 μl of the filtrate was derivatized with methylchloroformate, and the produced HTML was quantified using ion-pair UPLC-tandem mass spectrometry essentially as previously described (Vaz et al., 2002).

For determination of carnitine biosynthesis metabolites in brain homogenates, plasma, and urine, internal standards were added to each homogenate and derivatization was performed as described above. Brain homogenates and plasma samples were deproteinized using a Amicon Ultra 30-kDa filter. Urine samples were directly derivatized and TML, HTML, carnitine and γ-BB were quantified using ion-pair UPLC-tandem mass spectrometry as previously described (Vaz et al., 2002). For immunoblot analysis, a Multiphor II Nova Blot electrophoretic transfer unit (Amersham Pharmacia Biotech) was used to transfer proteins onto a nitrocellulose membrane (Whatman Protran, Dassel, Germany) as described by the manufacturer. After blocking of non specific binding sites with 50 g/l Protifar and 10 g/l BSA in PBS with Tween 20 (1 g/l) for 1 h, the membrane was incubated for 2 h in the same buffer without Protifar with 1:3000 dilution of rabbit polyclonal antibodies raised against human recombinant TMLD fused to maltose binding-protein (Vaz et al., 2001). Detection was performed with IRDye 800-conjugated goat anti-rabbit antibody (LICOR Biosciences) according to the manufacturers' instructions. Membranes were then dried and scanned using the Odyssey Infrared Imaging System (LI-COR).

### 450K Array Analysis

DNA from unaffected and autism cerebellum samples were treated with bisulfite using EZ DNA methylation kit (Zymo Research, Irvine, Calif., USA) and analyzed on the Infinium Human 450K Methylation arrays (Illumina, San Diego, Calif., USA). Average beta values that represent the extent of methylation for the interrogated CpG sites were plotted for all the samples.

### Brain Samples

Frozen human cerebellum samples were obtained from 11 autistic individuals and 53 unaffected individuals either from the Eunice Kennedy Shriver National Institute of Child Health and Human Development (NICHD) brain and tissue bank for developmental disorders at the University of Maryland, Baltimore, Md. (see World Wide Web) or from the Harvard brain tissue resource center (see World Wide Web) (Table below). Genomic DNA was extracted from the cerebellum samples by SDS-proteinase K digestion followed by phenol:chloroform extractions and ethanol precipitation. All brain samples were tested for presence or absence of exon 2 by PCR (see PCR methods below) and no deletions were detected.

Origin of Cerebellum Brain Samples

| ID | Gender | Phenotype | Collection for cerebellum samples |
|---|---|---|---|
| 1182 | F | Autism | NICHD brain and tissue bank for |
| | | | developmental disorders |
| 4231 | M | Autism | NICHD brain and tissue bank for |
| | | | developmental disorders |
| 7014 | M | Autism | Harvard brain tissue resource center |
| 19511 | M | Autism | Harvard brain tissue resource center |
| B4498 | M | Autism | Harvard brain tissue resource center |
| B4925 | M | Autism | Harvard brain tissue resource center |
| B5000 | M | Autism | Harvard brain tissue resource center |
| B5144 | M | Autism | Harvard brain tissue resource center |
| B5173 | M | Autism | Harvard brain tissue resource center |
| B6399 | M | Autism | Harvard brain tissue resource center |
| B6677 | M | Autism | Harvard brain tissue resource center |
| 54 | F | Normal | NICHD brain and tissue bank for |
| | | | developmental disorders |
| 771 | M | Normal | NICHD brain and tissue bank for |
| | | | developmental disorders |
| 814 | M | Normal | NICHD brain and tissue bank for |
| | | | developmental disorders |
| 1011 | M | Normal | NICHD brain and tissue bank for |
| | | | developmental disorders |
| 1025 | M | Normal | NICHD brain and tissue bank for |
| | | | developmental disorders |
| 1027 | M | Normal | NICHD brain and tissue bank for |
| | | | developmental disorders |
| 1038 | F | Normal | NICHD brain and tissue bank for |
| | | | developmental disorders |
| 1063 | M | Normal | NICHD brain and tissue bank for |
| | | | developmental disorders |
| 1079 | F | Normal | Harvard brain tissue resource center |
| 1091 | M | Normal | Harvard brain tissue resource center |
| 1093 | M | Normal | Harvard brain tissue resource center |
| 1101 | F | Normal | NICHD brain and tissue bank for |
| | | | developmental disorders |
| 1124 | M | Normal | Harvard brain tissue resource center |
| 1127 | F | Normal | Harvard brain tissue resource center |
| 1138 | F | Normal | Harvard brain tissue resource center |
| 1185 | M | Normal | NICHD brain and tissue bank for |
| | | | developmental disorders |
| 1259 | M | Normal | NICHD brain and tissue bank for |
| | | | developmental disorders |
| 1285 | M | Normal | NICHD brain and tissue bank for |
| | | | developmental disorders |
| 1324 | M | Normal | NICHD brain and tissue bank for |
| | | | developmental disorders |
| 1377 | F | Normal | NICHD brain and tissue bank for |
| | | | developmental disorders |
| 1407 | F | Normal | NICHD brain and tissue bank for |
| | | | developmental disorders |
| 1409 | M | Normal | NICHD brain and tissue bank for |
| | | | developmental disorders |
| 1429 | M | Normal | NICHD brain and tissue bank for |
| | | | developmental disorders |
| 1442 | M | Normal | NICHD brain and tissue bank for |
| | | | developmental disorders |
| 1453 | M | Normal | NICHD brain and tissue bank for |
| | | | developmental disorders |
| 1455 | F | Normal | NICHD brain and tissue bank for |
| | | | developmental disorders |
| 1465 | M | Normal | NICHD brain and tissue bank for |
| | | | developmental disorders |
| 1486 | F | Normal | NICHD brain and tissue bank for |
| | | | developmental disorders |
| 1488 | M | Normal | NICHD brain and tissue bank for |
| | | | developmental disorders |
| 1500 | M | Normal | NICHD brain and tissue bank for |
| | | | developmental disorders |
| 1547 | M | Normal | NICHD brain and tissue bank for |
| | | | developmental disorders |
| 1670 | M | Normal | NICHD brain and tissue bank for |
| | | | developmental disorders |
| 1674 | M | Normal | NICHD brain and tissue bank for |
| | | | developmental disorders |
| 1712 | M | Normal | NICHD brain and tissue bank for |
| | | | developmental disorders |
| 1713 | M | Normal | NICHD brain and tissue bank for |
| | | | developmental disorders |
| 1860 | M | Normal | NICHD brain and tissue bank for |
| | | | developmental disorders |
| 1906 | M | Normal | NICHD brain and tissue bank for |
| | | | developmental disorders |
| 1977 | M | Normal | NICHD brain and tissue bank for |
| | | | developmental disorders |
| 1981 | M | Normal | NICHD brain and tissue bank for |
| | | | developmental disorders |
| 2840 | M | Normal | NICHD brain and tissue bank for |
| | | | developmental disorders |
| 2880 | M | Normal | NICHD brain and tissue bank for |
| | | | developmental disorders |
| 3216 | M | Normal | NICHD brain and tissue bank for |
| | | | developmental disorders |
| 3237 | M | Normal | NICHD brain and tissue bank for |
| | | | developmental disorders |
| 3428 | F | Normal | NICHD brain and tissue bank for |
| | | | developmental disorders |
| 3455 | M | Normal | NICHD brain and tissue bank for |
| | | | developmental disorders |
| 4270 | M | Normal | NICHD brain and tissue bank for |
| | | | developmental disorders |
| 4271 | M | Normal | NICHD brain and tissue bank for |
| | | | developmental disorders |
| 4332 | M | Normal | NICHD brain and tissue bank for |
| | | | developmental disorders |
| 4337 | M | Normal | NICHD brain and tissue bank for |
| | | | developmental disorders |
| 4503 | F | Normal | NICHD brain and tissue bank for |
| | | | developmental disorders |
| 4670 | M | Normal | NICHD brain and tissue bank for |
| | | | developmental disorders |
| 5180 | M | Normal | NICHD brain and tissue bank for |
| | | | developmental disorders |
| 5282 | M | Normal | NICHD brain and tissue bank for |
| | | | developmental disorders |

### Lymphoblast Cultures

Lymphoblast cell lines (LCLs) from AGRE and SSC individuals were obtained from the Rutgers University Cell and DNA Repository. LCL from NA12003 (GM12003) (McCarroll et al., 2006) was obtained from the Coriell Institute for Medical Research (see World Wide Web site). LCLs from adult normal Caucasian Baylor Polymorphism Resource individuals (BPR) were obtained from the John W. Belmont laboratory tissue culture core, Department of Molecular and Human Genetics, Baylor College of Medicine, Houston, Tex., USA.

### RNA Extraction

RNA extraction from lymphoblast pellets was performed using Qiagen's miRNeasy Mini Kit (Qiagen, Germantown, Md.) following the manufacturer's instructions.

### Complementary DNA Conversion

For RT-PCR studies, one microgram of RNA was converted to cDNA using Invitrogen's Superscript III First Strand kit (Invitrogen, Carlsbad, Calif.) with 50 ng/uL random hexamers, following the manufacturer's instructions. For qRT-PCR studies, 100-500 ng of RNA was converted to cDNA using the High Capacity RNA-to-cDNA Master Kit (Applied Biosystems; Part#43907778) using the manufacturer's instructions. The cDNA product (20 ul) was diluted 4-fold and used for the qRT-PCR for each assay in triplicate.

### Quantitative RT-PCR

Analysis of the levels of gene expression for TMLHE exons were performed using TaqMan Gene Expression Assays (Applied Biosystems): exon boundary 1-2 (Hs00942999_ml) and 5-6 (Hs00379460_ml). Expression levels were endogenously normalized using TaqMan assays for genes ACTB (Hs00357333_g1). Each reaction contained 1 ul of cDNA, 1× assay (primer mix), 1×TaqMan Universal PCR Mastermix (Applied Biosystems; Part#4324018), and H2O in total volume of 10 ul. qRT-PCR was performed in 384-well plates using the Applied Biosystems 7900HT Real-Time PCR System via standard cycling conditions: 95° C. for 10 minutes, and 40 cycles of 95° C. for 15 seconds and 60° C. for 1 minutes. The data was analyzed using the SDS RQ Manager software (Applied Biosystems) using the ΔΔCt protocol, after normalizing the expression level to the endogenous controls (ΔΔCt refers to data normalized endogenously to ACTB and then each assay was normalized to HI0688).

### Polymerase Chain Reaction (PCR), RT-PCR and Sequencing

PCR to amplify TMLHE exons 1-8 was performed using Roche FastStart Taq DNA Polymerase, dNTPack (Roche Diagnostics, Mannheim, Germany). Briefly, 50-100 ng of genomic DNA were used in a 25 μl reaction that also contained 0.25 μM primers (Integrated DNA Technologies, Inc., Coralville, Iowa) (Table below), 200 μM of each dNTP, 1 unit of FastStart Taq DNA Polymerase, and 10×PCR buffer. The PCR was performed with the following reaction conditions: 95° C. for 6 minute; 40 cycles of 95° C. for 30 seconds, 58° C. for 30 seconds, and 72° C. for 1 minute; and final extension at 72° C. for 7 minutes. The PCR products were purified and sequenced using standard protocol at a commercial service laboratory (GeneWiz Inc, South Plainfield, N.J.).

Long Range (LR) PCR to confirm the TMLHE loss and to sequence the deletion junction in several individuals was performed using Takara's LA PCR kit (Takara Bio, Inc., Shiga, Japan). Briefly, 50-100 ng of genomic DNA were used in a 25 μl reaction that also contained 0.5 μM primers (Integrated DNA Technologies, Inc., Coralville, Iowa) (Table below), 400 μM of each dNTP, 1.25 units of Takara LA Taq, and 10×LA PCR buffer. The PCR was performed with the following reaction conditions: 94° C. for 1 minute; 35 cycles of 94° C. for 30 seconds and 68° C. for 30-60 seconds/expected kilo-bases of extended DNA; 72° C. for 10 minutes. PCR products were purified from the agarose gel using the Wizard® SV Gel and PCR Clean-Up System (Promega, Madison, Wis.) and sent for nucleotide sequencing by Sanger di-deoxynucleotide sequencing (Macrogen USA, Rockville, Md.).

PCR for SLC22A5, exons 1-10, was performed using Roche FastStart Taq DNA Polymerase, dNTPack (Roche Diagnostics, Mannheim, Germany). Briefly, 50 ng of genomic DNA were used in a 25 μL reaction with total concentration of 0.2 uM of forward and reverse primers (Integrated DNA Technologies, Inc., Coralville, Iowa) (Table below), 0.16 mM dNTPs, 1 unit of FastStart Taq DNA Polymerase, and 1×PCR buffer, and 1×GC-rich solution. The PCR was performed with the following reaction conditions: 95° C. for 5 minute; 10 cycles of 94° C. for 45 seconds, 65° C. for 45 seconds, and 72° C. for 2 minute, with a 1° C. step down for extension at each cycle to a final 55° C.; 25 cycles of 94° C. for 45 seconds, 55° C. for 45 seconds, and 72° C. for 2 minute, and final extension at 72° C. for 7 minutes. The PCR products were purified and sequenced using standard protocol at a commercial service laboratory (GeneWiz Inc, South Plainfield, N.J.).

PCR to detect exon 2 skipping in LCL complementary DNA (cDNA) of AGRE AU 0177 individuals was performed using Roche's FastStart Taq DNA Polymerase, dNTPack (Roche Diagnostics, Mannheim, Germany). Briefly, 50-100 ng of cDNA were used in a 25 μL reaction that also contained 0.25 μM primers (Integrated DNA Technologies, Inc., Coralville, Iowa) (Table below), 200 μM of each dNTP, 1 unit of FastStart Taq DNA Polymerase, and 10×PCR buffer. The PCR was performed with the following reaction conditions: 95° C. for 6 minute; 40 cycles of 95° C. for 30 seconds, 54° C. for 30 seconds, and 72° C. for 1 minute; and final extension at 72° C. for 7 minutes.

### Multiplex Ligation-Dependent Probe Amplification (MLPA)

Probes for MLPA analysis were designed using the freely available H-MAPD Web server (see World Wide Web site) within all 10 exons of SLC22A5 (OTCN2) (Table below). MLPA reactions were carried out using SALSA MLPA reagents and P300 reference probe mix as per instructions (MRC-Holland, Amsterdam). MLPA product (1.1 ml) and 0.25 ml of GS500 Liz SizeStandard was added to 10 ml of formamide and loaded onto an ABI 3730xl capillary electrophoresis machine (Applied Biosystems, Foster City, Calif.). Data were analyzed using GeneMarker MLPA analysis software (SoftGenetics, State College, Pa.).

### Multiplex Ligation-Dependent Probe Amplification (MLPA)

Probes for MLPA analysis were designed using the freely available H-MAPD Web server (see World Wide Web site) within all 10 exons of SLC22A5 (OTCN2) (Table 3 below). MLPA reactions were carried out using SALSA MLPA reagents and P300 reference probe mix as per instructions (MRC-Holland, Amsterdam). MLPA product (1.1 ml) and 0.25 ml of GS500 Liz SizeStandard was added to 10 ml of formamide and loaded onto an ABI 3730xl capillary electrophoresis machine (Applied Biosystems, Foster City, Calif.). Data were analyzed using GeneMarker MLPA analysis software (SoftGenetics, State College, Pa.).

TABLE 3

| Primer name | Gene/Exon | Assay | Primer sequence (5′-3′) |
|---|---|---|---|
| TMLHE Af | TMLHE 2 | LR PCR | CTACAGAAACAGAGAGATGGCAGTATCACAG (SEQ ID NO: 1) |
| TMLHE Ar | TMLHE 2 | LR PCR | GTGGTGATTCCTCAAGGATCTACAACTAGAAT (SEQ ID NO: 2) |
| TMLHE Bf | TMLHE 2 | LR PCR | TTTCTCCTAATGCTATCCCTCCCCTAGACC (SEQ ID NO: 3) |
| TMLHE Br | TMLHE 2 | LR PCR | GATGTGCAAGACCTCTATACTTGCTATTGTCA (SEQ ID NO: 4) |
| TMLHE Cf | TMLHE 2 | LR PCR | GTCTTGAGCTTTACAGGAGAGAAGCAGAATG (SEQ ID NO: 5) |
| TMLHE Cr | TMLHE 2 | LR PCR | GATGTGCAAGACCTCTATACTTGCTATTGTCA (SEQ ID NO: 6) |
| TMLHE Df | TMLHE 2 | LR PCR | CTCCAGAAAGCACCCCTACAGACATAGAGA (SEQ ID NO: 7) |
| TMLHE Dr | TMLHE 2 | LR PCR | CTCTCATCTTCTAGAGACTGCCTGCCTTAC (SEQ ID NO: 8) |
| TMLHE Ef | TMLHE 2 | LR PCR | TTTCTCCTAATGCTATCCCTCCCCTAGACC (SEQ ID NO: 9) |
| TMLHE Er | TMLHE 2 | LR PCR | GCACTTGAGGGAGGAAGATAAATGGAGTTAAC (SEQ ID NO: 10) |
| TMLHE 1F | TMLHE 1 | PCR | GTAAAACGACGGCCAGTAACAGAAGCTCCTCCAGTGC (SEQ ID NO: 11) |
| TMLHE 1R | TMLHE 1 | PCR | CAGGAAACAGCTATGACCGCCAGGGTTAAGGTGAGTA (SEQ ID NO: 12) |
| TMLHE 2Fc | TMLHE 2 | PCR | GTAAAACGACGGCCAGTCAATTTCTCCTTTGTTTAATCCTC (SEQ ID NO: 13) |
| TMLHE 2Rc | TMLHE 2 | PCR | CAGGAAACAGCTATGACGGTCCTGTCAACTCCACAAAA (SEQ ID NO: 14) |
| TMLHE 3F | TMLHE 3 | PCR | GTAAAACGACGGCCAGTCCCCTCAACCCTTATTGCTT (SEQ ID NO: 15) |
| TMLHE 3R | TMLHE 3 | PCR | CAGGAAACAGCTATGACCTTGCCATGTTCCCTGTCTT (SEQ ID NO: 16) |
| TMLHE 4F | TMLHE 4 | PCR | GTAAAACGACGGCCAGTTCATGCATACCACTCAAACCA (SEQ ID NO: 17) |
| TMLHE 4R | TMLHE 4 | PCR | CAGGAAACAGCTATGACCGAGGGTGACCATACCTGTT (SEQ ID NO: 18) |
| TMLHE 5F | TMLHE 5 | PCR | GTAAAACGACGGCCAGTTCCACAGTTTTCTCCCCAAC (SEQ ID NO: 19) |
| TMLHE 5R | TMLHE 5 | PCR | CAGGAAACAGCTATGACCTCCCCAGAAGAAGATGCTG (SEQ ID NO: 20) |
| TMLHE 6F | TMLHE 6 | PCR | GTAAAACGACGGCCAGTCAGGGCCCAGCACATATAA (SEQ ID NO: 21) |
| TMLHE 6R | TMLHE 6 | PCR | CAGGAAACAGCTATGACTTCTTTGGTTGGTGGCTATTT (SEQ ID NO: 22) |
| TMLHE 7F | TMLHE 7 | PCR | GTAAAACGACGGCCAGTACCTCAGCTTGGTGAGGAGA (SEQ ID NO: 23) |
| TMLHE 7R | TMLHE 7 | PCR | CAGGAAACAGCTATGACGATGAGGCTTGCTTCCAGAG (SEQ ID NO: 24) |
| TMLHE 8F | TMLHE 8 | PCR | GTAAAACGACGGCCAGTAACAGAAGCTCCTCCAGTGC (SEQ ID NO: 25) |
| TMLHE 8R | TMLHE 8 | PCR | CAGGAAACAGCTATGACTTTGTGGGATGGGATAAGGA (SEQ ID NO: 26) |
| TMLHE rt1Fb | TMLHE1 | RT-PCR | CTCCTTGCTTGGAATTCTGC (SEQ ID NO: 27) |
| TMLHE rt4R | TMLHE4 | RT-PCR | TTCAGTCCCTCGTTGGTTTC (SEQ ID NO: 28) |
| SLC22A5-P1F- | SLC22A5 | PCR | CTCGTTGTAAAACGACGGCCAGTAGCTTCGCGGCCGCTCTCCC (SEQ ID NO: 29) |
| | promoter | | |
| SLC22A5-P2R- | SLC22A5 | PCR | CTGCTCAGGAAACAGCTATGACCTGGCGCTGAGCAGGAAGAAGATGAGG |
| | promoter | | (SEQ ID NO: 30) |
| SLC22A5-E1F- | SLC22A5 1 | PCR | CTCGTTGTAAAACGACGGCCAGTCCTCCGCGGACGGTCTT (SEQ ID NO: 31) |
| SLC22A5-E1R- | SLC22A5 1 | PCR | CTGCTCAGGAAACAGCTATGACCGTCTCCATCGCTAGGGTGTT (SEQ ID NO: 32) |
| SLC22A5-E2F- | SLC22A5 2 | PCR | CTCGTTGTAAAACGACGGCCAGTAACACTTACTCTCTGCCTGTCTCTC |
| | | | (SEQ ID NO: 37) |
| SLC22A5-E2R- | SLC22A5 2 | PCR | CTGCTCAGGAAACAGCTATGACCTCACAGATACATTCAGCTTAGACAG |
| | | | (SEQ ID NO: 38) |
| SLC22A5-E3F- | SLC22A5 3 | PCR | CTCGTTGTAAAACGACGGCCAGTTCTTGAGAAAGCCCCACTTG (SEQ ID NO: 39) |
| SLC22A5-E3R- | SLC22A5 3 | PCR | CTGCTCAGGAAACAGCTATGACCAGGTTCACCAGGAAGCTCTG (SEQ ID NO: 40) |
| SLC22A5-E4F- | SLC22A5 4 | PCR | CTCGTTGTAAAACGACGGCCAGTCAGTCAGACAGGAGGGGTTC (SEQ ID NO: 41) |
| SLC22A5-E4R- | SLC22A5 4 | PCR | CTGCTCAGGAAACAGCTATGACCAATCTATGCTTCCTGTCTCTGG |
| | | | (SEQ ID NO: 42) |
| SLC22A5-E5F- | SLC22A5 5 | PCR | CTCGTTGTAAAACGACGGCCAGTCACAAGCTCTGGTTCTGCAA (SEQ ID NO: 43) |
| SLC22A5-E5R- | SLC22A5 5 | PCR | CTGCTCAGGAAACAGCTATGACCCTGTGAGCAGGGAGGACTTC (SEQ ID NO: 44) |
| SLC22A5-E6F- | SLC22A5 6 | PCR | CTCGTTGTAAAACGACGGCCAGTGCTGAGATGCAGACAGCTAA (SEQ ID NO: 45) |
| SLC22A5-E6R- | SLC22A5 6 | PCR | CTGCTCAGGAAACAGCTATGACCTTTTGTCTGAGGCATGTCTGT (SEQ ID NO: 46) |
| SLC22A5-E7F- | SLC22A5 7 | PCR | CTCGTTGTAAAACGACGGCCAGTGCAGGGTTACAGTTACTGCTG |
| | | | (SEQ ID NO: 47) |
| SLC22A5-E7R- | SLC22A5 7 | PCR | CTGCTCAGGAAACAGCTATGACCTAAGCAAGGGAGCTGTGATG (SEQ ID NO: 48) |
| SLC22A5-E8F- | SLC22A5 8 | PCR | CTCGTTGTAAAACGACGGCCAGTGATAGAAACTGACTCCCCAAAAA |
| | | | (SEQ ID NO: 49) |
| SLC22A5-E8R- | SLC22A5 8 | PCR | CTGCTCAGGAAACAGCTATGACCCACTGGGACAAGAACCCTTC (SEQ ID NO: 50) |
| SLC22A5-E9F- | SLC22A5 9 | PCR | CTCGTTGTAAAACGACGGCCAGTTGTGAGACCAAGAAGGAAAGTG |
| | | | (SEQ ID NO: 51) |
| SLC22A5-E9R- | SLC22A5 9 | PCR | CTGCTCAGGAAACAGCTATGACCGGCTACTGCCATGGAGATGT (SEQ ID NO: 52) |
| SLC22A5-E10F- | SLC22A5 10 | PCR | CTCGTTGTAAAACGACGGCCAGTCTGCTGCAGGATTCTCTTCC (SEQ ID NO: 53) |
| SLC22A5-E10R- | SLC22A5 10 | PCR | CTGCTCAGGAAACAGCTATGACCTGCTTCTCACATTCCTCCAG (SEQ ID NO: 54) |
| OTCN2_EX1_LS | SLC22A5 1 | MLPA | GGGTTCCCTAAGGGTTGGACTGCCGGGTGCCGGACGCCGCGAACCTGAGCAGC |
| | | | GCCTGGCGCAACCA (SEQ ID NO: 33) |
| OTCN2_EX1_RS | SLC22A5 1 | MLPA | CACTGTCCCACTGCGGCTGCGGGACGGCCGCGAGGTGCCCCACAGCTGTCTAG |
| | | | ATTGGATCTTGCTGGCAC (SEQ ID NO: 34) |
| OTCN2_EX2_LS | SLC22A5 2 | MLPA | GGGTTCCCTAAGGGTTGGAATTTCAGGGCAGCTGTCAGACAGGTAAGGTG |
| | | | (SEQ ID NO: 35) |
| OTCN2_EX2_RS | SLC22A5 2 | MLPA | TCTGTCTTCTGGAGCACCAGGGGACCTCAGCTCTAGATTGGATCTTGCTGGCAC |
| | | | (SEQ ID NO: 36) |
| OTCN2_EX3_LS | SLC22A5 3 | MLPA | GGGTTCCCTAAGGGTTGGAGTCACTCTCCTTTTCTTCCCAGGTTTGGCCGGAAG |
| | | | AATGT (SEQ ID NO: 64) |
| OTCN2_EX3_RS | SLC22A5 3 | MLPA | GCTGTTCGTGACCATGGGCATGCAGACAGGCTTCAGCTTCTCTAGATTGGATCT |
| | | | TGCTGGCAC (SEQ ID NO: 65) |
| OTCN2_EX4_LS | SLC22A5 4 | MLPA | GGGTTCCCTAAGGGTTGGACTGCTAACTCGACCTCCCTTGTTTTGAACAGGGAC |
| | | | AGAAATTCTTG (SEQ ID NO: 66) |
| OTCN2_EX4_RS | SLC22A5 4 | MLPA | GCAAGTCAGTTCGTATAATATTCTCTACGTTAGGAGTGTGCATATTTCTAGATT |
| | | | GGATCTTGCTGGCAC (SEQ ID NO: 67) |
| OTCN2_EX5_LS | SLC22A5 5 | MLPA | GGGTTCCCTAAGGGTTGGATGGGTGAGAGGGACAGACTGACCGTGATTTGAGA |
| | | | GC (SEQ ID NO: 68) |
| OTCN2_EX5_RS | SLC22A5 5 | MLPA | AGCAGCACCCAGCCCTGAAGTCCTCCCTGCTCACAGTCTAGATTGGATCTTGCT |
| | | | GGCAC (SEQ ID NO: 69) |
| OTCN2_EX6_LS | SLC22A5 6 | MLPA | GGGTTCCCTAAGGGTTGGACAGTCCCACAACATTCTGGATCTGCTTCG |
| | | | (SEQ ID NO: 70) |
| OTCN2_EX6_RS | SLC22A5 6 | MLPA | AACCTGGAATATCCGGATGGTCACCATCATCTAGATTGGATCTTGCTGGCAC |
| | | | (SEQ ID NO: 71) |
| OTCN2_EX7_LS | SLC22A5 7 | MLPA | GGGTTCCCTAAGGGTTGGACCCCGGCGCTATTCCATGGCCACTGCCCTCTTCCT |
| | | | GGGTGGCAGTGTCCT (SEQ ID NO: 72) |
| OTCN2_EX7_RS | SLC22A5 7 | MLPA | TCTCTTCATGCAGCTGGTACCCCCAGGTAGGGACCATGTGCATCTATGGTTCTA |
| | | | GATTGGATCTTGCTGGCAC (SEQ ID NO: 73) |
| OTCN2_EX8_LS | SLC22A5 8 | MLPA | GGGTTCCCTAAGGGTTGGATGCAGGTCGAAAAATGGGTGGATGGCGAAAGTGC |
| | | | ATGTCCTTATAGCTGCAGGTC (SEQ ID NO: 74) |
| OTCN2_EX8_RS | SLC22A5 8 | MLPA | CCAGCCTCCTTTCAGCAATCAATTTGGAGAGGAAAGCAATACTCTGGGACACGT |
| | | | ATCTAGATTGGATCTTGCTGGCAC (SEQ ID NO: 75) |
| OTCN2_EX9_LS | SLC22A5 9 | MLPA | GGGTTCCCTAAGGGTTGGATAAGAAGACCTCCTCTGTCAGTGTTGATGCACTGG |
| | | | GTC (SEQ ID NO: 76) |
| OTCN2_EX9_RS | SLC22A5 9 | MLPA | TGGGTCTGGCCAGGTCTCAGGAGCCCCTCACAATAGAGTCTAGATTGGATCTTG |
| | | | CTGGCAC (SEQ ID NO: 77) |
| OTCN2_EX10_LS | SLC22A5 10 | MLPA | GGGTTCCCTAAGGGTTGGATATTACGCCCGTGCCTTATCCGGAGAGGGCAGATC |
| | | | ACCAGCAAAGTGCACTGATGTG (SEQ ID NO: 78) |
| OTCN2_EX10_RS | SLC22A5 10 | MLPA | TGAGCTCTTAAGACCACTCAGCATGACGAATGAATGACGCGACAGGAAGAACT |
| | | | TGACTCTAGATTGGATCTTGCTGGCAC (SEQ ID NO: 79) |

### Tissue Culture

Lymphoblast cell lines were cultured in RPMI 1640 medium with L-Glutamine (Lonza, Basel, Switzerland), 10% fetal bovine serum (Thermo Fisher Scientific, Waltham, Mass.) and 1% Penicillin/Streptomycin solution (Thermo Fisher Scientific, Waltham, Mass.). Cultures were incubated at 37° C. in a 5% carbon dioxide water-jacketed incubator (Plymouth, Minn.). Cultures were grown in T75 tissue culture flasks (Greiner Bio-One, Monroe, N.C.) for 5 days in 40-50 mL medium. For RNA extraction, 50 mL of the confluent culture were spun down for 10 minutes at 1,000 rpm, the medium was aspirated off, and the pellet was frozen at −80° C. For protein assays, 50 mL of the confluent culture were spun down for 10 minutes at 1,000 rpm, the medium was aspirated off, and the pellet was washed twice in 15 mL sterile 1× phosphate buffered saline (PBS) without calcium and magnesium (Cellgro by Mediatech, Inc., Manassas, Va.) following centrifugation for 10 minutes at 1,000 rpm. The pellet was re-suspended in 1 mL PBS, transferred to a 1.5 mL Eppendorf tube, and spun for 3 minutes at 1,000 rpm. The liquid was decanted and the pellet was frozen at −80° C.

### Linkage Analyses

Nonparametric linkage analysis was performed using samples from the Autism Genetic Resource Exchange (AGRE) data center which were genotyped at the Children's Hospital of Philadelphia (CHOP) using the Illumina Infinium HumanHap 550 BeadChip. Only those families with at least two autistic male children with no known chromosomal abnormalities were included in the analysis. A total of 411 families consisting of 1,961 individuals with available genotype data were analyzed. Since it was hypothesized that three genes, TMLHE (Xq26), VAMP7 [Xq28 (pseudoautosomal region)] and SLC22A5 (5q31.1) could be potentially be involved in the genetic etiology of autism, we analyzed all available SNPs within the two megabase region which encompassed the TMLHE and SLC22A5 genes and all available SNP marker loci within the pseudoautosomal region for the VAMP7 gene. Quality control of genotype data was performed using PEDCHECK (O'Connell and Weeks, 1998) to identify Mendelian inconsistencies and MERLIN (Abecasis et al., 2002) to detect occurrences of double recombination events over short genetic distances, which are most likely due to genotyping error. Genotype frequencies were estimated from the AGRE data but since all founders were genotyped these estimated frequencies had no impact on the resulting NPL and LOD scores. Genetic map distances according to the Rutgers combined linkage-physical map of the human genome Build 37 version (Matise et al., 2007) were used to carry out the multipoint analysis. For markers which are not on the Rutgers map, the physical map position from the human reference sequence (Build 37) was used to interpolate the genetic map positions. Multipoint non-parametric linkage analysis was performed using MERLIN for the gene regions located on the autosome (SLC22A5) and within pseudoautosomal region (VAMP7), and MINX, a version of Merlin which was developed specifically to perform linkage analysis of the X-chromosome, was used to analyze the TMLHE gene region. The results of the nonparametric analysis are based on the NPL-ALL scoring function (Whittemore and Halpern, 1994), which takes into consideration within pairs as well as between pair allele sharing for a given pedigree. The NLP scores and LOD scores that were both derived using Kong and Cox exponential and linear models (Kong and Cox, 1997).

### TABLES

TABLE S1
Related to FIG. 1

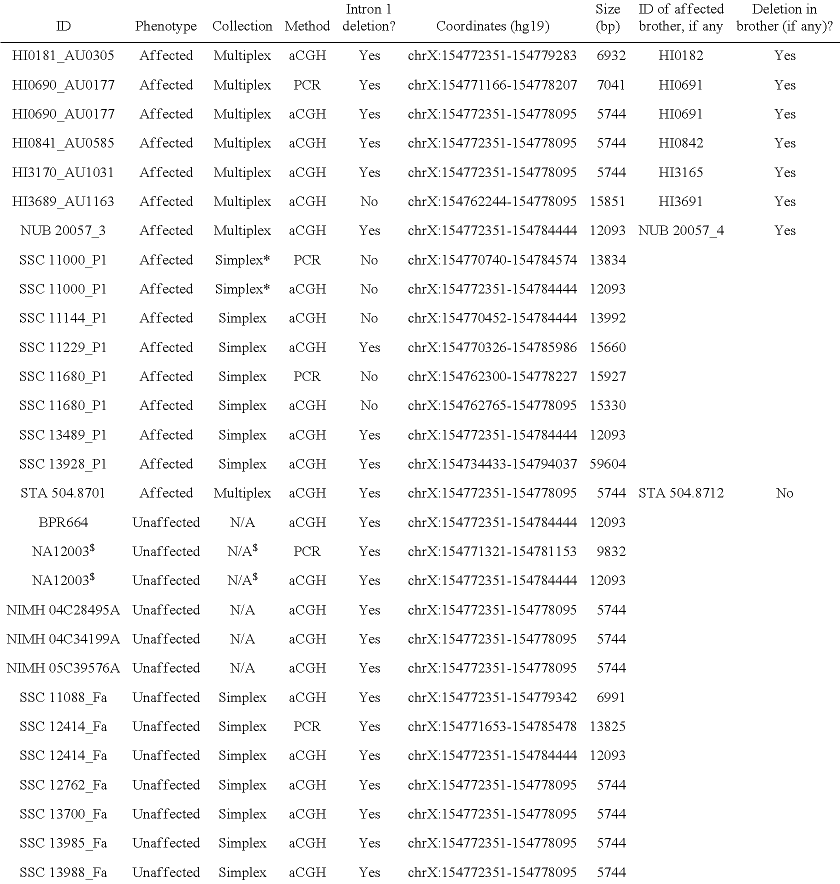

List of all TMLHE exon 2 deletions found and described in this study.
For multiplex families, the original brother detected by PCR is listed primarily.
Deletions in the other brother were identical.
Blue highlights refer to long-range PCR being used to determine breakpoints.
*Deletion first described in Celestino-Soper et al., unpublished data.
^{$}Deletion from (McCarroll et al., 2006). Breakpoint described in Celestino-Soper et al., unpublished data.
HI, individuals from AGRE families;
NUB and STA, families local to author JS;
SSC, Simons Simplex Collection;
BPR, individuals local to author ALB;
NIMH, National Institutes of Mental Health.

TABLE S2
Related to FIG. 1
Description of deletion breakpoints defined by PCR and sequencing.

| | | | Repeats at breakpoint^{c} | |
|---|---|---|---|---|
| ID | Sequence^{a} | Features^{b} | Left | Right |
| SSC 11000.p1 | L:TGCACGTGTACCCTAGAA (SEQ ID | 7 bp microhomology | LINE | LINE |
| | NO: 55) | | (L1PA7) | (L1PA6) |
| | S:TGCACGTGTACCCCAGAA (SEQ ID | | | |
| | NO: 56) | | | |
| | R:TGCACATGTACCCCAGAA (SEQ ID | | | |
| | NO: 57) | | | |
| NA12003(McCarroll et | L:TACACTCCCACCAACACTGT (SEQ ID | 11 bp microhomology | LINE | LINE |
| al., 2006) | NO: 80) | | (L1PA7) | (L1MC) |
| | S:TACACTCCCACCAACAGTG (SEQ ID | | | |
| | NO: 81) | | | |
| | R: TACATTCCCACCAACAGTGT (SEQ ID | | | |
| | NO: 82) | | | |
| SSC 12414.fa | L:TGCA...ACCT...CAGTCTAC (SEQ ID | 64 bp homology | LINE | LINE |
| | NO: 58) | | (L1PA7) | (L1PA6) |
| | S:TGCA...ACCT...CAATCTAT (SEQ ID | | | |
| | NO: 59) | | | |
| | R:TCCA...ACCT...CAATCTAT (SEQ ID | | | |
| | NO: 60) | | | |
| SSC 11680.p1 | L:GTTCTTGTCCTAGGGTGT (SEQ ID | 3 bp microhomology | None | SINE |
| | NO: 83) | | | (AluSg) |
| | S:GTTCTTGTCCTACTAAAA (SEQ ID | | | |
| | NO: 84) | | | |
| | R:ACCCTGTCTCTACTAAAA (SEQ ID | | | |
| | NO: 85) | | | |
| AU 0177 (HI0690 and | L:TAAACATATGTGTG (SEQ ID NO: 61) | No microhomology; extra | LINE | SINE |
| HI0691) | S:TAAACA-SINE-AACATGGT (SEQ ID | nucleotides | (L1PA7) | (AluSg) |
| | NO: 62) | | | |
| | R:CTGACTAACATGGT (SEQ ID NO: 63) | | | |

^{a}Bold: nucleotides in sequence results; underlined: nucleotides belonging to the microhomology segment; blue font: nucleotides in breakpoint region not present in the sequence results.
^{b}Features relates to number of basepairs with uninterrupted perfect microhomology.
^{c}Repeats present at breakpoints were obtained from UCSC genome browser (see World Wide Web).
ID, identification; L, left breakpoint region; S, fragment of sequenced PCR product containing breakpoints; R, right breakpoint region; LINE, long interspersed element; STR, simple tandem repeat.

TABLE S3
Part 1. Individuals assayed for exonic mutations in TMLHE
(exons 1, 6, and 8 lacked detectable mutations for these specific individuals)

| | | Exons | | | | | |
|---|---|---|---|---|---|---|---|
| Phenotype | Gender | 2 | 3 | 4 | 5 | 7 | Notes |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | 98886C > T:191F > F | no mt | no mt | silent mutation |
| | | | | (silent) | | | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | ex 2 deletion | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | 349V > I | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Autism | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | 88343G > A:74V > I | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | ex 2 deletion | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |
| Unaffected | M | no mt | no mt | no mt | no mt | no mt | |

TABLE S3
(Part 2)
Individuals assayed for exonic mutations in TMLHE

| AGRE | | DNA | Exon 1 through 8 |
|---|---|---|---|
| ID | Gender | origin | results |
| HI0020 | M | LCLs | No mutation |
| HI0054 | M | LCLs | No mutation |
| HI0079 | M | LCLs | No mutation |
| HI0088 | M | LCLs | No mutation |
| HI0105 | M | LCLs | No mutation |
| HI0120 | M | LCLs | No mutation |
| HI0134 | M | LCLs | No mutation |
| HI0150 | M | LCLs | No mutation |
| HI0168 | M | LCLs | No mutation |
| HI0186 | M | LCLs | No mutation |
| HI0197 | M | LCLs | No mutation |
| HI0205 | M | LCLs | No mutation |
| HI0210 | M | LCLs | No mutation |
| HI0229 | M | LCLs | No mutation |
| HI0266 | M | LCLs | No mutation |
| HI0274 | M | LCLs | No mutation |
| HI0301 | M | LCLs | No mutation |
| HI0328 | M | LCLs | No mutation |
| HI0332 | M | LCLs | No mutation |
| HI0413 | M | LCLs | No mutation |
| HI0437 | M | LCLs | No mutation |
| HI0489 | M | LCLs | No mutation |
| HI0496 | M | LCLs | No mutation |
| HI0515 | M | LCLs | No mutation |
| HI0530 | M | LCLs | No mutation |
| HI0542 | M | LCLs | No mutation |
| HI0545 | M | LCLs | No mutation |
| HI0550 | M | LCLs | No mutation |
| HI0564 | M | LCLs | No mutation |
| HI0587 | M | LCLs | No mutation |
| HI0591 | M | LCLs | No mutation |
| HI0603 | M | LCLs | No mutation |
| HI0613 | M | LCLs | No mutation |
| HI0619 | M | LCLs | No mutation |
| HI0675 | M | LCLs | No mutation |
| HI0682 | M | LCLs | No mutation |
| HI0690 | M | LCLs | No mutation |
| HI0705 | M | LCLs | No mutation |
| HI0725 | M | LCLs | No mutation |
| HI0730 | M | LCLs | No mutation |
| HI0745 | M | LCLs | No mutation |
| HI0757 | M | LCLs | No mutation |
| HI0774 | M | LCLs | No mutation |
| HI0777 | M | LCLs | No mutation |
| HI0803 | M | LCLs | No mutation |
| HI0830 | M | LCLs | No mutation |
| HI0871 | M | LCLs | No mutation |
| HI0876 | M | LCLs | No mutation |
| HI0894 | M | LCLs | No mutation |
| HI0955 | M | LCLs | No mutation |
| HI0976 | M | LCLs | No mutation |
| HI0994 | M | LCLs | No mutation |
| HI1004 | M | LCLs | No mutation |
| HI1012 | M | LCLs | No mutation |
| HI1030 | M | LCLs | No mutation |
| HI1066 | M | LCLs | No mutation |
| HI1073 | M | LCLs | No mutation |
| HI1076 | M | LCLs | No mutation |
| HI1082 | M | LCLs | No mutation |
| HI1097 | M | LCLs | No mutation |
| HI1102 | M | LCLs | No mutation |
| HI1116 | M | LCLs | No mutation |
| HI1129 | M | LCLs | No mutation |
| HI1139 | M | LCLs | No mutation |
| HI1152 | M | LCLs | No mutation |
| HI1169 | M | LCLs | No mutation |
| HI1183 | M | LCLs | No mutation |
| HI1190 | M | LCLs | No mutation |
| HI1199 | M | LCLs | No mutation |
| HI1220 | M | LCLs | No mutation |
| HI1243 | M | LCLs | No mutation |
| HI1261 | M | LCLs | No mutation |
| HI1266 | M | LCLs | No mutation |
| HI1278 | M | LCLs | No mutation |
| HI1291 | M | LCLs | No mutation |
| HI1302 | M | LCLs | No mutation |
| HI1341 | M | LCLs | No mutation |
| HI1357 | M | LCLs | No mutation |
| HI1364 | M | LCLs | No mutation |
| HI1459 | M | LCLs | No mutation |
| HI1492 | M | LCLs | No mutation |
| HI1500 | M | LCLs | No mutation |
| HI1507 | M | LCLs | No mutation |
| HI1733 | M | LCLs | No mutation |
| HI1765 | M | LCLs | No mutation |
| HI1893 | M | LCLs | No mutation |
| HI2013 | M | LCLs | No mutation |
| HI2071 | M | LCLs | No mutation |
| HI2091 | M | LCLs | No mutation |
| HI2322 | M | LCLs | No mutation |
| HI3392 | M | LCLs | No mutation |
| HI3402 | M | LCLs | No mutation |
| HI3422 | M | LCLs | No mutation |
| HI4269 | M | LCLs | No mutation |
| HI4605 | M | LCLs | No mutation |
| HI4944 | M | LCLs | No mutation |
| HI5863 | M | LCLs | No mutation |
| HI8357 | M | LCLs | No mutation |

HI AGRE identification;
M, male;
LCLs, lymphoblast cell lines.

TABLE S4
Related to FIG. 3

| | | | | | | TMLD activity |
|---|---|---|---|---|---|---|
| Sample ID | Phenotype | Tissue | Collection | Gender | Exon 2 genotype | (pmol/mg/min) |
| HI0032 | Autism | LCL | AGRE | M | No mutation | 25.1 |
| HI0080 | Autism | LCL | AGRE | M | No mutation | 52.3 |
| HI0115 | Autism | LCL | AGRE | M | No mutation | 45.2 |
| HI0120 | Autism | LCL | AGRE | M | No mutation | 34.7 |
| HI0437 | Autism | LCL | AGRE | M | No mutation | 27.7 |
| HI0505 | Autism | LCL | AGRE | M | No mutation | 30.3 |
| HI0671 | Autism | LCL | AGRE | M | No mutation | 27.7 |
| HI0690 | Autism | LCL | AGRE | M | Exon 2 deletion | 0.7 |
| HI0691 | Autism | LCL | AGRE | M | Exon 2 deletion | 0.1 |
| HI0733 | Autism | LCL | AGRE | M | No mutation | 44.9 |
| HI0955 | Autism | LCL | AGRE | M | No mutation | 65.1 |
| HI0962 | Autism | LCL | AGRE | M | No mutation | 44.0 |
| HI0976 | Autism | LCL | AGRE | M | No mutation | 51.4 |
| HI1066 | Autism | LCL | AGRE | M | No mutation | 27.4 |
| HI1073 | Autism | LCL | AGRE | M | No mutation | 53.3 |
| HI1097 | Autism | LCL | AGRE | M | No mutation | 23.7 |
| HI1102 | Autism | LCL | AGRE | M | No mutation | 28.9 |
| HI1129 | Autism | LCL | AGRE | M | No mutation | 22.9 |
| HI1170 | Autism | LCL | AGRE | M | No mutation | 37.4 |
| HI1190 | Autism | LCL | AGRE | M | No mutation | 48.1 |
| HI1243 | Autism | LCL | AGRE | M | No mutation | 33.9 |
| HI1376 | Autism | LCL | AGRE | M | No mutation | 28.9 |
| SSC 11000.p1 | Autism | LCL | SSC | M | Exon 2 deletion | 0.33 |
| SSC 11680.p1 | Autism | LCL | SSC | M | Exon 2 deletion | −1.9 |
| SSC 12353.p1 | Autism | LCL | SSC | M | R241Q | 15.1 |
| BPR 086 | Unaffected | LCL | BPR | M | No mutation | 34.9 |
| BPR 156 | Unaffected | LCL | BPR | M | No mutation | 38.8 |
| BPR 251 | Unaffected | LCL | BPR | M | No mutation | 23.36 |
| BPR001 | Unaffected | LCL | BPR | M | No mutation | 17.8 |
| BPR046 | Unaffected | LCL | BPR | M | No mutation | 15.2 |
| BPR057 | Unaffected | LCL | BPR | M | No mutation | 43.6 |
| BPR059 | Unaffected | LCL | BPR | M | No mutation | 22.6 |
| BPR080 | Unaffected | LCL | BPR | M | No mutation | 30.3 |
| BPR088 | Unaffected | LCL | BPR | M | No mutation | 27.6 |
| BPR111 | Unaffected | LCL | BPR | M | No mutation | 48.3 |
| BPR151 | Unaffected | LCL | BPR | M | No mutation | 42.6 |
| BPR157 | Unaffected | LCL | BPR | M | No mutation | 12.5 |
| BPR162 | Unaffected | LCL | BPR | M | No mutation | 24.0 |
| BPR172 | Unaffected | LCL | BPR | M | No mutation | 30.2 |
| BPR176 | Unaffected | LCL | BPR | M | No mutation | 38.2 |
| BPR190 | Unaffected | LCL | BPR | M | No mutation | 32.7 |
| BPR234 | Unaffected | LCL | BPR | M | No mutation | 56.8 |
| BPR254 | Unaffected | LCL | BPR | M | No mutation | 32.5 |
| BPR266 | Unaffected | LCL | BPR | M | No mutation | 25.6 |
| BPR401 | Unaffected | LCL | BPR | M | No mutation | 26.4 |
| BPR575 | Unaffected | LCL | BPR | F | No mutation | 86.6 |
| BPR654 | Unaffected | LCL | BPR | M | No mutation | 37.4 |
| BPR655 | Unaffected | LCL | BPR | F | No mutation | 38.1 |
| BPR662 | Unaffected | LCL | BPR | M | No mutation | 30.9 |
| BPR664 | Unaffected | LCL | BPR | M | Exon 2 deletion | 0.5 |
| NA 12003 | Unaffected | LCL | CEPH | M | Exon 2 deletion | 0.6 |
| SSC 11000.fa | Unaffected | LCL | SSC | M | No mutation | 25.39 |
| SSC 11000.mo | Unaffected | LCL | SSC | F | Het exon 2 deletion | 16.15 |
| SSC 11680.fa | Unaffected | LCL | SSC | M | No mutation | 20.66 |
| SSC 11680.mo | Unaffected | LCL | SSC | F | Het exon 2 deletion | 11.05 |
| SSC 12353.fa | Unaffected | LCL | SSC | M | No mutation | 28.49 |
| SSC 12353.mo | Unaffected | LCL | SSC | F | Het R241Q | 6.91 |
| SSC 12353.s1 | Unaffected | LCL | SSC | F | Het R241Q | 17.67 |
| SSC 12414.fa | Unaffected | LCL | SSC | M | Exon 2 deletion | −3.62 |
| HI0688 | Unaffected_Fa | LCL | AGRE | M | No exon mutation | 25.6 |
| HI0689 | Unaffected_Mo | LCL | AGRE | F | Het exon2 deletion; | 3.5 |
| | | | | | Het R241Q | |
| HI0692 | Unaffected_S1 | LCL | AGRE | M | R241Q | 3.7 |

TMLD activity in lymphoblast cell line of various autism and unaffected individuals.
P1, proband;
fa, father;
mo, mother;
s1, sibling;
LCL, in lymphoblast cell line;
M, male;
F, female;
Het, heterozygous.

TABLE S5
Related to FIG. 3 and Table 1.

| | | | | Metabolite levels | | | | | |
|---|---|---|---|---|---|---|---|---|---|
| | | | TMLD activity | (pmol/mg protein) | | | | | |
| ID | Phenotype | Gender | (pmol/mg/min) | TML | HTML | Carnitine | γBB | TML/HTML | TML/γBB |
| 1182 | Autism | F | 19 | 362 | 21 | 1475 | 52 | 17 | 7 |
| 4231 | Autism | M | 24 | 111 | 10 | 344 | 39 | 11 | 3 |
| 7014 | Autism | M | 15 | 248 | 10 | 354 | 43 | 26 | 6 |
| 19511 | Autism | M | 62 | 202 | 13 | 778 | 79 | 15 | 3 |
| B4925 | Autism | M | 32 | 133 | 13 | 572 | 46 | 10 | 3 |
| B5000 | Autism | M | 31 | 237 | 16 | 1297 | 53 | 15 | 4 |
| B5144 | Autism | M | 24 | 199 | 9 | 486 | 30 | 22 | 7 |
| B5173 | Autism | M | 19 | 156 | 6 | 452 | 22 | 26 | 7 |
| B6399 | Autism | M | 20 | 160 | 18 | 608 | 63 | 9 | 3 |
| B6677 | Autism | M | 25 | 106 | 5 | 485 | 37 | 22 | 3 |
| 754 | Unaffected | F | 46 | 191 | 9 | 597 | 20 | 22 | 9 |
| 771 | Unaffected | M | 37 | 207 | 15 | 465 | 42 | 14 | 5 |
| 814 | Unaffected | M | 30 | 112 | 16 | 654 | 22 | 7 | 5 |
| 1011 | Unaffected | M | 37 | 153 | 8 | 653 | 19 | 18 | 8 |
| 1025 | Unaffected | M | 32 | 181 | 10 | 679 | 38 | 19 | 5 |
| 1027 | Unaffected | M | 37 | 324 | 15 | 705 | 29 | 22 | 11 |
| 1038 | Unaffected | F | 23 | 287 | 14 | 818 | 90 | 20 | 3 |
| 1063 | Unaffected | M | 24 | 212 | 22 | 764 | 57 | 10 | 4 |
| 1070 | Unaffected | M | 33 | 928 | 61 | 797 | 50 | 15 | 18 |
| 1079 | Unaffected | F | 45 | 81 | 11 | 530 | 31 | 8 | 3 |
| 1093 | Unaffected | M | 21 | 341 | 7 | 952 | 28 | 46 | 12 |
| 1101 | Unaffected | F | 45 | 104 | 13 | 535 | 45 | 8 | 2 |
| 1124 | Unaffected | M | 26 | 593 | 6 | 1053 | 30 | 102 | 20 |
| 1127 | Unaffected | F | 35 | 193 | 11 | 1129 | 36 | 18 | 5 |
| 1138 | Unaffected | F | 23 | 591 | 12 | 732 | 89 | 48 | 7 |
| 1185 | Unaffected | M | 15 | 567 | 16 | 1156 | 29 | 37 | 19 |
| 1259 | Unaffected | M | 31 | 228 | 12 | 1274 | 34 | 19 | 7 |
| 1285 | Unaffected | M | 26 | 269 | 10 | 797 | 38 | 28 | 7 |
| 1324 | Unaffected | M | 33 | 213 | 8 | 557 | 15 | 26 | 14 |
| 1377 | Unaffected | F | 29 | 324 | 15 | 705 | 29 | 22 | 11 |
| 1407 | Unaffected | F | 47 | 413 | 13 | 664 | 29 | 31 | 14 |
| 1409 | Unaffected | M | 43 | 188 | 13 | 1074 | 41 | 15 | 5 |
| 1429 | Unaffected | M | 33 | 240 | 15 | 1477 | 56 | 16 | 4 |
| 1442 | Unaffected | M | 37 | 102 | 8 | 721 | 27 | 13 | 4 |
| 1453 | Unaffected | M | 47 | 106 | 16 | 882 | 43 | 7 | 2 |
| 1455 | Unaffected | F | 41 | 276 | 15 | 770 | 35 | 18 | 8 |
| 1486 | Unaffected | F | 34 | 141 | 10 | 760 | 27 | 14 | 5 |
| 1500 | Unaffected | M | 47 | 778 | 30 | 1087 | 41 | 26 | 19 |
| 1670 | Unaffected | M | 25 | 89 | 11 | 670 | 45 | 8 | 2 |
| 1860 | Unaffected | M | 67 | 462 | 22 | 508 | 21 | 21 | 22 |
| 1977 | Unaffected | M | 36 | 99 | 11 | 463 | 21 | 9 | 5 |
| 1981 | Unaffected | M | 25 | 304 | 19 | 1045 | 26 | 16 | 12 |
| 2840 | Unaffected | M | 27 | 108 | 14 | 699 | 27 | 8 | 4 |
| 2880 | Unaffected | M | 26 | 154 | 8 | 739 | 41 | 20 | 4 |
| 3216 | Unaffected | M | 29 | 111 | 4 | 1439 | 19 | 28 | 6 |
| 3237 | Unaffected | M | 18 | 84 | 7 | 513 | 20 | 11 | 4 |
| 3428 | Unaffected | F | 42 | 119 | 7 | 540 | 17 | 16 | 7 |
| 3455 | Unaffected | M | 30 | 124 | 10 | 1241 | 22 | 12 | 6 |
| 4270 | Unaffected | M | 40 | 111 | 10 | 684 | 27 | 11 | 4 |
| 4271 | Unaffected | M | 19 | 204 | 9 | 1461 | 43 | 23 | 5 |
| 4503 | Unaffected | F | 29 | 295 | 7 | 646 | 20 | 44 | 15 |
| 5180 | Unaffected | M | 33 | 228 | 39 | 805 | 33 | 6 | 7 |
| 5282 | Unaffected | M | 44 | 337 | 25 | 667 | 25 | 13 | 14 |

TMLD activity and metabolite measurements in cerebellum of various autism and unaffected individuals.
M, male;
F, female;
TML, 6-N-trimethyllysine;
HTML, 3-hydroxy-6-N-trimethyllysine;
γBB, γ-butyrobetaine (or 4-trimethylaminobutyric acid).

TABLE S6
Related to FIG. 1 and Table 2
Probands/cases

| Individual | VIQ/NVIQ/FSIQ^{a} | Age^{b} | Regression | Seizures | Language Level^{c} | Comments |
|---|---|---|---|---|---|---|
| SSC 11000.p1 | 50/78/65 | 9.2 | No | No | 0 | ADOS Mod 2 at age 9.2 |
| SSC 11144.p1 | 99/127/111 | 10.8 | No | No | 0 | ADOS Mod 3 at age 10.9 |
| SSC 11229.p1 | 56/68/63 | 9.4 | No | No | 0 | ADOS Mod 3 at age 9.4 |
| SSC 11680.p1 | 54/82/71 | 8 | No | No | 0 | ADOS Mod 3 at age 8.0 |
| SSC 13489.p1 | 94/99/97 | 7.5 | No | No | 0 | ADOS Mod 3 at age 7.5 |
| SSC 13928.p1 | 27/53/38 | 6.3 | No | No | 1 | ADOS Mod 1 at age 6.3 |
| HI0691 (AU 0177-03) | — | 4.9 | Yes^{d} | Yes^{e} | 2 | ADOS Mod 1 at age 7.3 |
| HI0690 (AU 0177-04) | 92/—/— | 2.9 | No | No | 2 | ADOS Mod 2 at age 5.2 |
| HI3170 (AU 1031-301) | 111/122/— | 7.8 | No | — | 0 | ADOS Mod 3 at age 7.8 |
| HI 3165 (AU 1031-303) | —/untestable/— | 4.3 | No | — | 2 | ADOS Mod 1 at age 4.3 |
| HI3689 (AU 1163-304) | —/88/— | 8.4 | No | — | 0 | ADOS Mod 2 at age 8.2 |
| HI3691 (AU 1163-305) | —/110/— | 6.9 | No | — | 1 | ADOS Mod 1 at age 6.9 |
| HI0842 (AU-0585-03) | 92/80/— | 4.5 | No | — | 0 | ADOS Mod 3 at age 5.7 |
| HI0841 (AU-0585-04) | —/untestable/— | 3.2 | No | No | 2 | ADOS Mod 1 at age 4.4 |
| HI0181 (AU 0305-03) | 121/114/— | 7.7 | No | No | 0 | ADOS Mod 3 at age 11.1 |
| HI0182 (AU 0305-04) | —/untestable/— | 6 | No | No | 2 | ADOS Mod 1 at age 9.4 |
| NUB20057-3 (NIMH-152-MM0057-3) | 158/128/143 | 6 | No | — | 0 | — |
| NUB20057-4 (NIMH-152-MM0057-4) | 87/120/104 | 3 | No | — | 1 | — |
| STA504-8701 (NIMH-63-8701) | —/44/— | 4 | No | — | 2 | — |
| STA504_8712 (NIMH-63-8712) | —/45/— | 4 | No | — | 0 | No Exon 2 deletion |

Fathers/controls

| Individual | Highest Education^{f} | Age^{b} | BAP-Q^{g} | SRS-ARV^{h} | FHI-Iⁱ | Comments |
|---|---|---|---|---|---|---|
| SSC 11088_Fa | BA/BS | 48.7 | 4.75/2.33/4.00/3.69 | 15 | 0 | — |
| SSC 12414_Fa | BA/BS | 39.1 | 1.17/2.58/1.17/1.64 | 18 | 0 | — |
| SSC 12762_Fa | Grad/professional | 40 | 3.17/1.25/4.00/2.81 | 19 | 0 | — |
| SSC 13700_Fa | Associate | 40.3 | 3.25/1.92/2.33/2.50 | 37 | 0 | — |
| SSC 13985_Fa | BA/BS | 49.8 | 1.50/1.67/2.50/1.89 | 36 | 2 | — |
| SSC 13988_Fa | Associate | 35.3 | 1.58/2.92/2.42/2.31 | 11 | 0 | — |
| HI0692 (AU 0177-05) | — | — | — | — | — | Unaffected Half brother to AU- |
| | | | | | | 0177-03 and AU 0177-04 |
| NIMH 04C28495A | MA/MS | 60 | — | — | — | Alcoholism, max BMI 38.7 |
| (NIMH 150-10442) | | | | | | |
| NIMH 04C34199A | Associate | 52 | — | — | — | Prescription sedative/painkiller |
| (NIMH 150-11126) | | | | | | dependence, max BMI 21.0 |
| NIMH 05C39576A | Some college | 63 | — | — | — | Max BMI 35.3 |
| (NIMH 150-12165) | | | | | | |
| NA12003 | — | 97 | — | — | — | — |
| BPR664 | — | — | — | — | — | — |

Phenotypic information on affected and control males.
In the “Individuals” column,
“SSC” denotes a Simons Simplex Collection participant,
“AU” denotes an Autism Genetic Resource Exchange participant, and
“NIMH” denotes a National Institutes of Mental Health participant.
^{a}VIQ = Verbal IQ, NVIQ = Nonverbal IQ, FSIQ = Full-scale IQ. For all SSC cases, VIQ, NVIQ, and FSIQ scores were derived from either the Differential Abilities Scales - Second Edition or the Mullen Scales of Early Learning. For all AU cases, VIQ scores represent standard scores from the Peabody Picture Vocabulary Test, while NVIQ scores represent standard scores from the Raven Colored Progressive Matrices.
^{b}Age for probands/cases = age (in years) at the time of Autism Diagnostic Interview - Revised (ADI-R) evaluation; age for fathers/controls = age (in years) at the time of evaluation.
^{c}Language level represents overall language abilities from the ADI-R, where 0 = functional use of at least three-word phrases on a daily basis, 1 = speech used daily but not meeting criteria for a 0 code, and 2 = fewer than five words total or speech not used daily.
^{d}Regression was a non-language skill loss.
^{e}Seizure type was non-febrile seizures.
^{f}Associate = Associate degree; BA/BS = baccalaureate degree; Grad/professional = graduate school or professional degree; Some college = some college without graduation.
^{g}BAP-Q = Broad Autism Phenotype Questionnaire; average domain scores are listed as Aloof/Pragmatic Language/Rigid/Overall.
^{h}SRS-ARV = Social Responsiveness Scale - Adult Research Version; total score.
ⁱFHI-I = Interviewer's Impression of Interviewee form (International Molecular Genetic Study of Autism Consortium); total score. All NIMH controls were married (one now widowed), and all “enjoy meeting new people.” - , data not available or not collected.

TABLE S7
Related to Table S8

| | | | | |
|---|---|---|---|---|
| | | | Family | Refer- |
| SSC ID | Exon | [Sequence]/AA Change | mutation | ences |

Transmitted known deleterious

| | | | | |
|---|---|---|---|---|
| 12302.p1 | 1 | c.[364G > T]/p.D122DY | P, (F no seq) | A |
| 12383.p1 | 1 | c.[136C > T]/p.P46PS | P, F, S | A |
| 12520.p1 | 1 | c.[59T > A]/p.L20LH | P, F, S | A |
| 11567.p1 | 1 | c.[59T > A]/p.L20LH | P (M no seq) | A |
| 11198.p1 | 2, 9 | p.A142AS, p.R488RH | P, F, S | L, A |
| 12534,p1 | 4 | c.[695C > T]/p.T232TM | P, F, S | L, A |
| 12272.p1 | 7 | p.S362SL | P, F, S | L |

Non-transmitted known deleterious

| | | | | |
|---|---|---|---|---|
| 12652.fa | 1 | c.[136C > T]/p.P46PS | F, S | A |
| 11722.fa | 2 | c.[458_459het_delTG]/fs | M | Novel |
| 11522.mo | 7 | p.L394del | M, S | A |
| 11567.fa | 8 | p.Y449YD | F, S | L |

SLC22A5 mutations in 277 SSC families with regression in proband.
P, proband;
F, father;
M, mother;
S, sibling;
A, ARUP database (see World Wide Web site);
L, (Longo et al., 2006)

TABLE S8
Related to Table S7

| SSC | | SLC22A5 |
|---|---|---|
| Sample | SLC22A5 sequencing | MLPA |
| 11109_p1 | No mutation | No mutation |
| 11161_p1 | No mutation | No mutation |
| 11290_p1 | No mutation | No mutation |
| 11321_p1 | No mutation | No mutation |
| 11380_p1 | No mutation | No mutation |
| 11390_p1 | No mutation | No mutation |
| 11393_p1 | No mutation | No mutation |
| 11437_p1 | No mutation | No mutation |
| 11109_mo | No mutation | No mutation |
| 11161_mo | No mutation | No mutation |
| 11290_mo | No mutation | No mutation |
| 11321_mo | No mutation | No mutation |
| 11380_mo | No mutation | No mutation |
| 11390_mo | No mutation | No mutation |
| 11393_mo | No mutation | No mutation |
| 11437_mo | No mutation | No mutation |
| 11109_fa | No mutation | No mutation |
| 11161_fa | No mutation | No mutation |
| 11290_fa | No mutation | No mutation |
| 11321_fa | No mutation | No mutation |
| 11380_fa | No mutation | No mutation |
| 11390_fa | No mutation | No mutation |
| 11393_fa | No mutation | No mutation |
| 11437_fa | No mutation | No mutation |
| 11109_s1 | No mutation | No mutation |
| 11161_s1 | No mutation | No mutation |
| 11290_s1 | No mutation | No mutation |
| 11321_s1 | No mutation | No mutation |
| 11380_s1 | No mutation | No mutation |
| 11390_s1 | No mutation | No mutation |
| 11393_s1 | No mutation | NO DATA |
| 11437_s1 | No mutation | No mutation |
| 11518_p1 | No mutation | No mutation |
| 11543_p1 | No mutation | No mutation |
| 11573_p1 | No mutation | No mutation |
| 11608_p1 | No mutation | No mutation |
| 11620_p1 | No mutation | No mutation |
| 11634_p1 | p.A90AV/p.L144LF | No mutation |
| 11641_p1 | No mutation | No mutation |
| 11653_p1 | No mutation | NO DATA |
| 11518_mo | No mutation | No mutation |
| 11543_mo | No mutation | No mutation |
| 11573_mo | No mutation | No mutation |
| 11608_mo | No mutation | No mutation |
| 11620_mo | No mutation | No mutation |
| 11634_mo | p.A90AV/p.M177MT | No mutation |
| 11641_mo | No mutation | No mutation |
| 11653_mo | No mutation | No mutation |
| 11518_fa | No mutation | No mutation |
| 11543_fa | No mutation | No mutation |
| 11573_fa | No mutation | No mutation |
| 11608_fa | No mutation | No mutation |
| 11620_fa | No mutation | No mutation |
| 11634_fa | p.L144LF | No mutation |
| 11641_fa | No mutation | No mutation |
| 11653_fa | No mutation | No mutation |
| 11518_s1 | No mutation | No mutation |
| 11543_s1 | No mutation | No mutation |
| 11573_s1 | No mutation | No mutation |
| 11608_s1 | No mutation | No mutation |
| 11620_s1 | No mutation | No mutation |
| 11634_s1 | p.A90AV/p.L144LF | No mutation |
| 11641_s1 | No mutation | No mutation |
| 11653_s1 | No mutation | NO DATA |
| 11665_p1 | No mutation | No mutation |
| 11667_p1 | No mutation | No mutation |
| 11722_p1 | No mutation | No mutation |
| 11765_p1 | No mutation | No mutation |
| 11788_p1 | No mutation | No mutation |
| 11798_p1 | No mutation | No mutation |
| 11824_p1 | No mutation | No mutation |
| 11843_p1 | No mutation | No mutation |
| 11665_mo | No mutation | No mutation |
| 11667_mo | No mutation | No mutation |
| 11722_mo | c.[458_459het_delTG]/Frameshift | No mutation |
| 11765_mo | No mutation | No mutation |
| 11788_mo | No mutation | No mutation |
| 11798_mo | No mutation | No mutation |
| 11824_mo | No mutation | No mutation |
| 11843_mo | No mutation | No mutation |
| 11665_fa | No mutation | No mutation |
| 11667_fa | No mutation | No mutation |
| 11722_fa | No mutation | No mutation |
| 11765_fa | No mutation | NO DATA |
| 11788_fa | No mutation | No mutation |
| 11798_fa | No mutation | No mutation |
| 11824_fa | No mutation | No mutation |
| 11843_fa | No mutation | No mutation |
| 11665_s1 | No mutation | No mutation |
| 11667_s1 | No mutation | No mutation |
| 11722_s1 | No mutation | No mutation |
| 11765_s1 | No mutation | No mutation |
| 11788_s1 | No mutation | No mutation |
| 11798_s1 | No mutation | No mutation |
| 11868_p1 | No mutation | No mutation |
| 11868_mo | No mutation | No mutation |
| 11868_fa | No mutation | No mutation |
| 11868_s1 | No mutation | No mutation |
| 12284_p1 | No mutation | No mutation |
| 12284_mo | No mutation | No mutation |
| 12284_fa | No mutation | No mutation |
| 12284_s1 | No mutation | No mutation |
| 11990_p1 | No mutation | No mutation |
| 11990_mo | No mutation | No mutation |
| 11990_fa | No mutation | No mutation |
| 11990_s1 | No mutation | No mutation |
| 12302_p1 | p.D122DY | No mutation |
| 12302_mo | No mutation | No mutation |
| 12302_fa | NO SEQUENCE | NO DATA |
| 12302_s1 | No mutation | No mutation |
| 12029_p1 | No mutation | No mutation |
| 12029_mo | No mutation | No mutation |
| 12029_fa | No mutation | No mutation |
| 12029_s1 | No mutation | No mutation |
| 12325_p1 | No mutation | No mutation |
| 12325_mo | No mutation | No mutation |
| 12325_fa | No mutation | No mutation |
| 12325_s1 | No mutation | No mutation |
| 12071_p1 | No mutation | No mutation |
| 12071_mo | No mutation | No mutation |
| 12071_fa | No mutation | No mutation |
| 12071_s1 | No mutation | No mutation |
| 12326_p1 | No mutation | No mutation |
| 12326_mo | No mutation | No mutation |
| 12326_fa | No mutation | No mutation |
| 12326_s1 | No mutation | No mutation |
| 12150_p1 | No mutation | No mutation |
| 12150_mo | No mutation | No mutation |
| 12150_fa | No mutation | No mutation |
| 12150_s1 | No mutation | No mutation |
| 12383_p1 | p.P46PS/p.G484GV | No mutation |
| 12383_mo | p.G484GV | No mutation |
| 12383_fa | p.P46PS | No mutation |
| 12383_s1 | p.P46PS/p.G484GV | No mutation |
| 12180_p1 | No mutation | No mutation |
| 12180_mo | No mutation | No mutation |
| 12180_fa | No mutation | No mutation |
| 12180_s1 | No mutation | No mutation |
| 12385_p1 | No mutation | No mutation |
| 12385_mo | No mutation | No mutation |
| 12385_fa | No mutation | No mutation |
| 12385_s1 | No mutation | No mutation |
| 12187_p1 | No mutation | No mutation |
| 12187_mo | No mutation | No mutation |
| 12187_fa | No mutation | No mutation |
| 12187_s1 | No mutation | No mutation |
| 12441_p1 | No mutation | No mutation |
| 12441_mo | No mutation | No mutation |
| 12441_fa | No mutation | No mutation |
| 12441_s1 | No mutation | No mutation |
| 12221_p1 | p.V527VL | No mutation |
| 12221_mo | p.V527VL | No mutation |
| 12221_fa | No mutation | No mutation |
| 12221_s1 | No mutation | No mutation |
| 12447_p1 | No mutation | No mutation |
| 12447_mo | No mutation | No mutation |
| 12447_fa | No mutation | No mutation |
| 12447_s1 | No mutation | No mutation |
| 12227_p1 | No mutation | NO DATA |
| 12227_mo | No mutation | No mutation |
| 12227_fa | No mutation | No mutation |
| 12227_s1 | No mutation | No mutation |
| 12460_p1 | No mutation | No mutation |
| 12460_mo | No mutation | No mutation |
| 12460_fa | No mutation | No mutation |
| 12460_s1 | No mutation | No mutation |
| 12241_p1 | No mutation | NO DATA |
| 12241_mo | No mutation | No mutation |
| 12241_fa | No mutation | NO DATA |
| 12241_s1 | No mutation | No mutation |
| 12514_p1 | No mutation | No mutation |
| 12514_mo | No mutation | No mutation |
| 12514_fa | No mutation | No mutation |
| 12514_s1 | No mutation | No mutation |
| 12261_p1 | p.P549PS | No mutation |
| 12261_mo | No mutation | No mutation |
| 12261_fa | p.P549PS | No mutation |
| 12261_s1 | No mutation | No mutation |
| 12518_p1 | No mutation | No mutation |
| 12518_mo | No mutation | No mutation |
| 12518_fa | No mutation | No mutation |
| 12518_s1 | No mutation | No mutation |
| 12281_p1 | No mutation | No mutation |
| 12281_mo | No mutation | NO DATA |
| 12281_fa | No mutation | NO DATA |
| 12281_s1 | No mutation | No mutation |
| 11812_p1 | No mutation | No mutation |
| 11812_mo | No mutation | No mutation |
| 11812_fa | No mutation | No mutation |
| 11812_s1 | No mutation | No mutation |
| 12621_p1 | No mutation | No mutation |
| 12621_mo | No mutation | No mutation |
| 12621_fa | No mutation | No mutation |
| 12621_s1 | No mutation | No mutation |
| 11829_p1 | No mutation | No mutation |
| 11829_mo | No mutation | No mutation |
| 11829_fa | No mutation | No mutation |
| 11829_s1 | No mutation | No mutation |
| 12622_p1 | No mutation | No mutation |
| 12622_mo | No mutation | No mutation |
| 12622_fa | p.Y454YH | No mutation |
| 12622_s1 | No mutation | No mutation |
| 11838_p1 | No mutation | No mutation |
| 11838_mo | No mutation | No mutation |
| 11838_fa | No mutation | No mutation |
| 11838_s1 | No mutation | No mutation |
| 12631_p1 | No mutation | No mutation |
| 12631_mo | No mutation | No mutation |
| 12631_fa | No mutation | No mutation |
| 12631_s1 | No mutation | No mutation |
| 12301_p1 | No mutation | No mutation |
| 12301_mo | No mutation | No mutation |
| 12301_fa | No mutation | No mutation |
| 12301_s1 | No mutation | No mutation |
| 12637_p1 | No mutation | No mutation |
| 12637_mo | No mutation | No mutation |
| 12637_fa | No mutation | No mutation |
| 12637_s1 | No mutation | No mutation |
| 12473_p1 | No mutation | No mutation |
| 12473_mo | No mutation | No mutation |
| 12473_fa | No mutation | No mutation |
| 12473_s1 | No mutation | No mutation |
| 12652_p1 | No mutation | No mutation |
| 12652_mo | No mutation | No mutation |
| 12652_fa | p.P46PS | No mutation |
| 12652_s1 | p.P46PS | No mutation |
| 12484_p1 | No mutation | No mutation |
| 12484_mo | No mutation | No mutation |
| 12484_fa | No mutation | No mutation |
| 12484_s1 | No mutation | No mutation |
| 12655_p1 | No mutation | No mutation |
| 12655_mo | No mutation | No mutation |
| 12655_fa | No mutation | No mutation |
| 12655_s1 | No mutation | No mutation |
| 12520_p1 | p.L20LH | NO DATA |
| 12520_mo | No mutation | No mutation |
| 12520_fa | p.L20LH/p.M417MV | No mutation |
| 12520_s1 | p.L20LH/p.M417MV | No mutation |
| 12667_p1 | No mutation | No mutation |
| 12667_mo | No mutation | No mutation |
| 12667_fa | No mutation | No mutation |
| 12667_s1 | No mutation | No mutation |
| 12532_p1 | No mutation | No mutation |
| 12532_mo | No mutation | No mutation |
| 12532_fa | No mutation | No mutation |
| 12532_s1 | No mutation | No mutation |
| 12674_p1 | No mutation | No mutation |
| 12674_mo | No mutation | No mutation |
| 12674_fa | No mutation | No mutation |
| 12674_s1 | p.T232TM | No mutation |
| 12534_p1 | p.T232TM | No mutation |
| 12534_mo | No mutation | No mutation |
| 12534_fa | p.T232TM | No mutation |
| 12534_s1 | No mutation | No mutation |
| 12682_p1 | No mutation | No mutation |
| 12682_mo | No mutation | No mutation |
| 12682_fa | No mutation | No mutation |
| 12682_s1 | No mutation | No mutation |
| 12536_p1 | No mutation | No mutation |
| 12536_mo | No mutation | No mutation |
| 12536_fa | No mutation | No mutation |
| 12536_s1 | No mutation | No mutation |
| 12703_p1 | No mutation | No mutation |
| 12703_mo | No mutation | No mutation |
| 12703_fa | No mutation | No mutation |
| 12703_s1 | No mutation | No mutation |
| 12565_p1 | No mutation | No mutation |
| 12565_mo | No mutation | No mutation |
| 12565_fa | No mutation | No mutation |
| 12565_s1 | No mutation | No mutation |
| 12715_p1 | No mutation | No mutation |
| 12715_mo | No mutation | No mutation |
| 12715_fa | No mutation | No mutation |
| 12715_s1 | No mutation | No mutation |
| 12616_p1 | No mutation | No mutation |
| 12616_mo | No mutation | No mutation |
| 12616_fa | No mutation | No mutation |
| 12616_s1 | No mutation | No mutation |
| 12731_p1 | No mutation | No mutation |
| 12731_mo | No mutation | No mutation |
| 12731_fa | No mutation | No mutation |
| 12731_s1 | No mutation | No mutation |
| 13017_p1 | No mutation | No mutation |
| 13017_mo | No mutation | No mutation |
| 13017_fa | No mutation | No mutation |
| 13017_s1 | No mutation | No mutation |
| 12799_p1 | No mutation | No mutation |
| 12799_mo | No mutation | No mutation |
| 12799_fa | No mutation | No mutation |
| 12799_s1 | No mutation | No mutation |
| 13028_p1 | No mutation | No mutation |
| 13028_mo | No mutation | No mutation |
| 13028_fa | No mutation | No mutation |
| 13028_s1 | No mutation | No mutation |
| 12817_p1 | No mutation | No mutation |
| 12817_mo | No mutation | No mutation |
| 12817_fa | No mutation | No mutation |
| 12817_s1 | No mutation | No mutation |
| 13035_p1 | No mutation | No mutation |
| 13035_mo | No mutation | No mutation |
| 13035_fa | No mutation | No mutation |
| 13035_s1 | No mutation | No mutation |
| 12821_p1 | No mutation | No mutation |
| 12821_mo | No mutation | No mutation |
| 12821_fa | No mutation | No mutation |
| 12821_s1 | No mutation | No mutation |
| 13051_p1 | No mutation | No mutation |
| 13051_mo | No mutation | No mutation |
| 13051_fa | No mutation | No mutation |
| 13051_s1 | p.A44AT | No mutation |
| 12833_p1 | No mutation | No mutation |
| 12833_mo | No mutation | No mutation |
| 12833_fa | No mutation | No mutation |
| 12833_s1 | No mutation | No mutation |
| 13061_p1 | No mutation | No mutation |
| 13061_mo | No mutation | No mutation |
| 13061_fa | No mutation | No mutation |
| 13061_s1 | No mutation | No mutation |
| 12843_p1 | No mutation | No mutation |
| 12843_mo | p.A44AT | No mutation |
| 12843_fa | No mutation | No mutation |
| 12843_s1 | No mutation | No mutation |
| 13069_p1 | No mutation | No mutation |
| 13069_mo | No mutation | No mutation |
| 13069_fa | No mutation | No mutation |
| 13069_s1 | No mutation | No mutation |
| 12857_p1 | No mutation | No mutation |
| 12857_mo | No mutation | No mutation |
| 12857_fa | No mutation | No mutation |
| 12857_s1 | No mutation | No mutation |
| 13088_p1 | No mutation | No mutation |
| 13088_mo | No mutation | No mutation |
| 13088_fa | No mutation | No mutation |
| 13088_s1 | No mutation | No mutation |
| 12864_p1 | No mutation | No mutation |
| 12864_mo | No mutation | No mutation |
| 12864_fa | No mutation | No mutation |
| 12864_s1 | No mutation | No mutation |
| 13129_p1 | No mutation | No mutation |
| 13129_mo | No mutation | No mutation |
| 13129_fa | No mutation | No mutation |
| 13129_s1 | No mutation | No mutation |
| 12875_p1 | No mutation | No mutation |
| 12875_mo | No mutation | No mutation |
| 12875_fa | No mutation | No mutation |
| 12875_s1 | No mutation | No mutation |
| 13136_p1 | p.V481VI | No mutation |
| 13136_mo | p.V481VI | No mutation |
| 13136_fa | No mutation | No mutation |
| 13136_s1 | No mutation | No mutation |
| 12974_p1 | No mutation | No mutation |
| 12974_mo | No mutation | No mutation |
| 12974_fa | No mutation | No mutation |
| 12974_s1 | No mutation | No mutation |
| 13143_p1 | No mutation | No mutation |
| 13143_mo | No mutation | No mutation |
| 13143_fa | No mutation | No mutation |
| 13143_s1 | No mutation | No mutation |
| 12981_p1 | No mutation | No mutation |
| 12981_mo | No mutation | No mutation |
| 12981_fa | No mutation | No mutation |
| 12981_s1 | No mutation | No mutation |
| 13147_p1 | No mutation | No mutation |
| 13147_mo | No mutation | No mutation |
| 13147_fa | No mutation | No mutation |
| 13147_s1 | No mutation | No mutation |
| 13008_p1 | No mutation | No mutation |
| 13008_mo | No mutation | No mutation |
| 13008_fa | No mutation | No mutation |
| 13008_s1 | No mutation | No mutation |
| 13175_p1 | No mutation | No mutation |
| 13175_mo | No mutation | No mutation |
| 13175_fa | No mutation | No mutation |
| 13175_s1 | No mutation | No mutation |
| 13499_p1 | No mutation | No mutation |
| 13499_mo | No mutation | No mutation |
| 13499_fa | No mutation | No mutation |
| 13499_s1 | No mutation | No mutation |
| 13207_p1 | No mutation | No mutation |
| 13207_mo | No mutation | No mutation |
| 13207_fa | No mutation | No mutation |
| 13207_s1 | No mutation | No mutation |
| 13504_p1 | No mutation | No mutation |
| 13504_mo | No mutation | No mutation |
| 13504_fa | No mutation | No mutation |
| 13504_s1 | No mutation | No mutation |
| 13266_p1 | No mutation | No mutation |
| 13266_mo | No mutation | No mutation |
| 13266_fa | No mutation | No mutation |
| 13266_s1 | No mutation | No mutation |
| 13515_p1 | No mutation | No mutation |
| 13515_mo | No mutation | No mutation |
| 13515_fa | No mutation | No mutation |
| 13515_s1 | No mutation | No mutation |
| 13290_p1 | No mutation | No mutation |
| 13290_mo | p.A44AT | No mutation |
| 13290_fa | No mutation | No mutation |
| 13290_s1 | p.A44AT | No mutation |
| 13517_p1 | No mutation | No mutation |
| 13517_mo | No mutation | No mutation |
| 13517_fa | No mutation | No mutation |
| 13517_s1 | No mutation | No mutation |
| 13306_p1 | No mutation | No mutation |
| 13306_mo | No mutation | No mutation |
| 13306_fa | No mutation | No mutation |
| 13306_s1 | No mutation | No mutation |
| 13518_p1 | No mutation | No mutation |
| 13518_mo | No mutation | No mutation |
| 13518_fa | No mutation | No mutation |
| 13518_s1 | No mutation | No mutation |
| 13348_p1 | No mutation | No mutation |
| 13348_mo | No mutation | No mutation |
| 13348_fa | No mutation | No mutation |
| 13348_s1 | No mutation | No mutation |
| 13529_p1 | p.R488RH | No mutation |
| 13529_mo | p.R488RH | No mutation |
| 13529_fa | No mutation | No mutation |
| 13529_s1 | p.R488RH | No mutation |
| 13349_p1 | No mutation | No mutation |
| 13349_mo | No mutation | No mutation |
| 13349_fa | No mutation | No mutation |
| 13349_s1 | No mutation | No mutation |
| 13533_p1 | No mutation | No mutation |
| 13533_mo | No mutation | No mutation |
| 13533_fa | No mutation | No mutation |
| 13533_s1 | No mutation | No mutation |
| 13355_p1 | No mutation | No mutation |
| 13355_mo | No mutation | No mutation |
| 13355_fa | No mutation | No mutation |
| 13355_s1 | No mutation | No mutation |
| 13535_p1 | No mutation | No mutation |
| 13535_mo | No mutation | No mutation |
| 13535_fa | No mutation | No mutation |
| 13535_s1 | No mutation | No mutation |
| 13385_p1 | No mutation | No mutation |
| 13385_mo | No mutation | No mutation |
| 13385_fa | No mutation | No mutation |
| 13385_s1 | No mutation | No mutation |
| 13539_p1 | No mutation | No mutation |
| 13539_mo | No mutation | No mutation |
| 13539_fa | No mutation | No mutation |
| 13539_s1 | No mutation | No mutation |
| 13390_p1 | No mutation | No mutation |
| 13390_mo | No mutation | No mutation |
| 13390_fa | No mutation | No mutation |
| 13390_s1 | No mutation | No mutation |
| 13540_p1 | No mutation | No mutation |
| 13540_mo | No mutation | No mutation |
| 13540_fa | No mutation | No mutation |
| 13540_s1 | No mutation | No mutation |
| 13444_p1 | No mutation | No mutation |
| 13444_mo | No mutation | No mutation |
| 13444_fa | No mutation | No mutation |
| 13444_s1 | No mutation | No mutation |
| 13546_p1 | No mutation | No mutation |
| 13546_mo | No mutation | No mutation |
| 13546_fa | No mutation | No mutation |
| 13546_s1 | No mutation | No mutation |
| 13456_p1 | No mutation | No mutation |
| 13456_mo | No mutation | No mutation |
| 13456_fa | No mutation | No mutation |
| 13456_s1 | No mutation | No mutation |
| 13555_p1 | No mutation | No mutation |
| 13555_mo | p.P549PS | No mutation |
| 13555_fa | No mutation | No mutation |
| 13555_s1 | p.P549PS | No mutation |
| 12300_p1 | No mutation | No mutation |
| 12300_mo | No mutation | No mutation |
| 12300_fa | No mutation | No mutation |
| 12300_s1 | No mutation | No mutation |
| 13556_p1 | No mutation | No mutation |
| 13556_mo | No mutation | No mutation |
| 13556_fa | No mutation | No mutation |
| 13556_s1 | No mutation | No mutation |
| 12366_p1 | No mutation | No mutation |
| 12366_mo | No mutation | No mutation |
| 12366_fa | No mutation | No mutation |
| 12366_s1 | No mutation | No mutation |
| 13574_p1 | No mutation | No mutation |
| 13574_mo | No mutation | No mutation |
| 13574_fa | No mutation | No mutation |
| 13574_s1 | No mutation | No mutation |
| 12624_p1 | No mutation | No mutation |
| 12624_mo | No mutation | No mutation |
| 12624_fa | No mutation | No mutation |
| 12624_s1 | No mutation | No mutation |
| 13581_p1 | No mutation | No mutation |
| 13581_mo | No mutation | No mutation |
| 13581_fa | No mutation | No mutation |
| 13581_s1 | No mutation | No mutation |
| 12638_p1 | No mutation | No mutation |
| 12638_mo | No mutation | No mutation |
| 12638_fa | No mutation | No mutation |
| 12638_s1 | p.M177MV | No mutation |
| 13608_p1 | No mutation | No mutation |
| 13608_mo | No mutation | No mutation |
| 13608_fa | No mutation | No mutation |
| 13608_s1 | No mutation | No mutation |
| 12648_p1 | No mutation | No mutation |
| 12648_mo | No mutation | No mutation |
| 12648_fa | No mutation | No mutation |
| 12648_s1 | No mutation | No mutation |
| 13612_p1 | No mutation | No mutation |
| 13612_mo | No mutation | No mutation |
| 13612_fa | No mutation | No mutation |
| 13612_s1 | No mutation | No mutation |
| 12661_p1 | No mutation | No mutation |
| 12661_mo | p.M381MT | No mutation |
| 12661_fa | No mutation | No mutation |
| 12661_s1 | p.M381MT | No mutation |
| 13615_p1 | No mutation | No mutation |
| 13615_mo | No mutation | No mutation |
| 13615_fa | No mutation | No mutation |
| 13615_s1 | No mutation | No mutation |
| 12708_p1 | No mutation | No mutation |
| 12708_mo | No mutation | No mutation |
| 12708_fa | No mutation | No mutation |
| 12708_s1 | No mutation | No mutation |
| 13631_p1 | No mutation | No mutation |
| 13631_mo | No mutation | No mutation |
| 13631_fa | No mutation | No mutation |
| 13631_s1 | No mutation | No mutation |
| 12763_p1 | No mutation | No mutation |
| 12763_mo | No mutation | No mutation |
| 12763_fa | No mutation | No mutation |
| 12763_s1 | No mutation | No mutation |
| 13683_p1 | No mutation | No mutation |
| 13683_mo | No mutation | No mutation |
| 13683_fa | No mutation | No mutation |
| 13683_s1 | No mutation | No mutation |
| 12858_p1 | No mutation | No mutation |
| 12858_mo | No mutation | No mutation |
| 12858_fa | No mutation | No mutation |
| 12858_s1 | No mutation | No mutation |
| 11055_p1 | No mutation | No mutation |
| 11055_mo | No mutation | No mutation |
| 11055_fa | No mutation | No mutation |
| 11055_s1 | No mutation | No mutation |
| 12871_p1 | No mutation | No mutation |
| 12871_mo | No mutation | No mutation |
| 12871_fa | No mutation | No mutation |
| 12871_s1 | No mutation | No mutation |
| 11873_p1 | No mutation | No mutation |
| 11873_mo | No mutation | No mutation |
| 11873_fa | No mutation | No mutation |
| 11873_s1 | No mutation | No mutation |
| 12906_p1 | No mutation | No mutation |
| 12906_mo | No mutation | No mutation |
| 12906_fa | No mutation | No mutation |
| 12906_s1 | No mutation | No mutation |
| 12285_p1 | No mutation | No mutation |
| 12285_mo | No mutation | No mutation |
| 12285_fa | No mutation | No mutation |
| 12285_s1 | No mutation | No mutation |
| 12937_p1 | No mutation | No mutation |
| 12937_mo | No mutation | No mutation |
| 12937_fa | No mutation | NO DATA |
| 12937_s1 | No mutation | No mutation |
| 13497_p1 | No mutation | No mutation |
| 13497_mo | No mutation | No mutation |
| 13497_fa | No mutation | No mutation |
| 13497_s1 | No mutation | No mutation |
| 12996_p1 | No mutation | No mutation |
| 12996_mo | No mutation | No mutation |
| 12996_fa | No mutation | No mutation |
| 12996_s1 | No mutation | No mutation |
| 11214_p1 | p.R488RH | NO DATA |
| 11214_mo | No mutation | No mutation |
| 11214_fa | p.R488RH | No mutation |
| 11214_s1 | p.R488RH | No mutation |
| 13042_p1 | No mutation | No mutation |
| 13042_mo | No mutation | No mutation |
| 13042_fa | No mutation | No mutation |
| 13042_s1 | No mutation | No mutation |
| 11375_p1 | No mutation | No mutation |
| 11375_mo | No mutation | No mutation |
| 11375_fa | No mutation | No mutation |
| 11375_s1 | No mutation | No mutation |
| 13211_p1 | No mutation | No mutation |
| 13211_mo | No mutation | No mutation |
| 13211_fa | No mutation | No mutation |
| 13211_s1 | No mutation | No mutation |
| 12578_p1 | No mutation | No mutation |
| 12578_mo | No mutation | No mutation |
| 12578_fa | No mutation | No mutation |
| 12578_s1 | No mutation | No mutation |
| 13216_p1 | No mutation | No mutation |
| 13216_mo | No mutation | No mutation |
| 13216_fa | No mutation | No mutation |
| 13216_s1 | No mutation | No mutation |
| 12792_p1 | No mutation | No mutation |
| 12792_mo | No mutation | No mutation |
| 12792_fa | No mutation | No mutation |
| 12792_s1 | No mutation | No mutation |
| 13332_p1 | No mutation | No mutation |
| 13332_mo | No mutation | No mutation |
| 13332_fa | No mutation | No mutation |
| 13332_s1 | No mutation | No mutation |
| 12922_p1 | No mutation | No mutation |
| 12922_mo | No mutation | No mutation |
| 12922_fa | No mutation | No mutation |
| 12922_s1 | No mutation | No mutation |
| 13345_p1 | No mutation | No mutation |
| 13345_mo | No mutation | No mutation |
| 13345_fa | No mutation | No mutation |
| 13345_s1 | No mutation | No mutation |
| 13093_p1 | No mutation | No mutation |
| 13093_mo | No mutation | No mutation |
| 13093_fa | No mutation | No mutation |
| 13093_s1 | No mutation | No mutation |
| 13371_p1 | No mutation | No mutation |
| 13371_mo | No mutation | No mutation |
| 13371_fa | No mutation | No mutation |
| 13371_s1 | No mutation | No mutation |
| 13097_p1 | No mutation | No mutation |
| 13097_mo | No mutation | No mutation |
| 13097_fa | p.A44AT | No mutation |
| 13097_s1 | No mutation | No mutation |
| 13532_p1 | No mutation | No mutation |
| 13532_mo | No mutation | No mutation |
| 13532_fa | No mutation | No mutation |
| 13532_s1 | No mutation | No mutation |
| 13107_p1 | No mutation | No mutation |
| 13107_mo | No mutation | No mutation |
| 13107_fa | No mutation | No mutation |
| 13107_s1 | No mutation | No mutation |
| 12994_p1 | No mutation | No mutation |
| 12994_mo | No mutation | NO DATA |
| 12994_fa | No mutation | No mutation |
| 12994_s1 | No mutation | No mutation |
| 13125_p1 | No mutation | No mutation |
| 13125_mo | No mutation | No mutation |
| 13125_fa | No mutation | No mutation |
| 13125_s1 | No mutation | No mutation |
| 13057_p1 | No mutation | No mutation |
| 13057_mo | No mutation | No mutation |
| 13057_fa | No mutation | No mutation |
| 13057_s1 | No mutation | No mutation |
| 13302_p1 | No mutation | No mutation |
| 13302_mo | No mutation | No mutation |
| 13302_fa | No mutation | No mutation |
| 13302_s1 | No mutation | No mutation |
| 13160_p1 | No mutation | No mutation |
| 13160_mo | No mutation | No mutation |
| 13160_fa | No mutation | No mutation |
| 13160_s1 | No mutation | No mutation |
| 13383_p1 | No mutation | No mutation |
| 13383_mo | No mutation | No mutation |
| 13383_fa | No mutation | No mutation |
| 13383_s1 | No mutation | No mutation |
| 11325_p1 | No mutation | No mutation |
| 11325_mo | No mutation | No mutation |
| 11325_fa | No mutation | No mutation |
| 11325_s1 | No mutation | No mutation |
| 11056_p1 | No mutation | No mutation |
| 11056_mo | No mutation | No mutation |
| 11056_fa | No mutation | No mutation |
| 11056_s1 | No mutation | No mutation |
| 11366_p1 | No mutation | No mutation |
| 11366_mo | No mutation | No mutation |
| 11366_fa | No mutation | No mutation |
| 11366_s1 | No mutation | No mutation |
| 11074_p1 | No mutation | No mutation |
| 11074_mo | No mutation | No mutation |
| 11074_fa | No mutation | No mutation |
| 11074_s1 | No mutation | No mutation |
| 11421_p1 | No mutation | No mutation |
| 11421_mo | No mutation | No mutation |
| 11421_fa | No mutation | No mutation |
| 11421_s1 | No mutation | No mutation |
| 11108_p1 | No mutation | No mutation |
| 11108_mo | No mutation | No mutation |
| 11108_fa | No mutation | No mutation |
| 11108_s1 | No mutation | No mutation |
| 11440_p1 | No mutation | No mutation |
| 11440_mo | No mutation | No mutation |
| 11440_fa | No mutation | No mutation |
| 11440_s1 | No mutation | No mutation |
| 11124_p1 | No mutation | No mutation |
| 11124_mo | No mutation | No mutation |
| 11124_fa | No mutation | No mutation |
| 11124_s1 | No mutation | No mutation |
| 11504_p1 | No mutation | No mutation |
| 11504_mo | No mutation | No mutation |
| 11504_fa | p.R488RH | No mutation |
| 11504_s1 | p.R488RH | No mutation |
| 11160_p1 | No mutation | No mutation |
| 11160_mo | No mutation | No mutation |
| 11160_fa | No mutation | No mutation |
| 11160_s1 | No mutation | No mutation |
| 11509_p1 | No mutation | No mutation |
| 11509_mo | No mutation | No mutation |
| 11509_fa | No mutation | No mutation |
| 11509_s1 | No mutation | No mutation |
| 11172_p1 | No mutation | No mutation |
| 11172_mo | No mutation | No mutation |
| 11172_fa | No mutation | No mutation |
| 11172_s1 | No mutation | No mutation |
| 11522_p1 | NO SEQUENCE | NO DATA |
| 11522_mo | p.L394del | No mutation |
| 11522_fa | No mutation | No mutation |
| 11522_s1 | p.L394del | No mutation |
| 11190_p1 | No mutation | No mutation |
| 11190_mo | No mutation | No mutation |
| 11190_fa | No mutation | No mutation |
| 11190_s1 | No mutation | No mutation |
| 11549_p1 | No mutation | No mutation |
| 11549_mo | p.A44AT | No mutation |
| 11549_fa | p.A44AT | No mutation |
| 11549_s1 | p.A44AT(homozygous) | No mutation |
| 11198_p1 | p.A142AS/p.R488RH/p.R488RH | No mutation |
| 11198_mo | No mutation | No mutation |
| 11198_fa | p.A142AS/p.R488RH/p.R488RH | No mutation |
| 11198_s1 | p.A142AS/p.R488RH/p.R488RH | No mutation |
| 11551_p1 | NO SEQUENCE | NO DATA |
| 11551_mo | NO SEQUENCE | NO DATA |
| 11551_fa | p.R488RH | No mutation |
| 11551_s1 | p.R488RH | No mutation |
| 11201_p1 | No mutation | No mutation |
| 11201_mo | No mutation | NO DATA |
| 11201_fa | No mutation | No mutation |
| 11201_s1 | No mutation | No mutation |
| 11564_p1 | No mutation | No mutation |
| 11564_mo | No mutation | No mutation |
| 11564_fa | p.A44AT | No mutation |
| 11564_s1 | p.A44AT | No mutation |
| 11206_p1 | No mutation | No mutation |
| 11206_mo | No mutation | No mutation |
| 11206_fa | No mutation | No mutation |
| 11206_s1 | No mutation | No mutation |
| 11567_p1 | p.L20LH/p.M417MV | No mutation |
| 11567_mo | NO SEQUENCE | NO DATA |
| 11567_fa | p.Y449YD | No mutation |
| 11567_s1 | p.Y449YD/p.P549PS | No mutation |
| 11245_p1 | No mutation | No mutation |
| 11245_mo | No mutation | No mutation |
| 11245_fa | No mutation | No mutation |
| 11245_s1 | No mutation | No mutation |
| 11040_p1 | No mutation | No mutation |
| 11040_mo | No mutation | No mutation |
| 11040_fa | No mutation | No mutation |
| 11040_s1 | No mutation | No mutation |
| 11671_p1 | No mutation | NO DATA |
| 11671_mo | No mutation | No mutation |
| 11671_fa | No mutation | No mutation |
| 11671_s1 | No mutation | No mutation |
| 11078_p1 | No mutation | No mutation |
| 11078_mo | No mutation | No mutation |
| 11078_fa | No mutation | No mutation |
| 11078_s1 | No mutation | No mutation |
| 11679_p1 | No mutation | No mutation |
| 11679_mo | No mutation | No mutation |
| 11679_fa | No mutation | No mutation |
| 11679_s1 | No mutation | No mutation |
| 11110_p1 | No mutation | No mutation |
| 11110_mo | No mutation | No mutation |
| 11110_fa | No mutation | No mutation |
| 11110_s1 | No mutation | No mutation |
| 11707_p1 | No mutation | No mutation |
| 11707_mo | No mutation | No mutation |
| 11707_fa | No mutation | NO DATA |
| 11707_s1 | No mutation | NO DATA |
| 11169_p1 | No mutation | No mutation |
| 11169_mo | No mutation | No mutation |
| 11169_fa | No mutation | No mutation |
| 11169_s1 | No mutation | No mutation |
| 11712_p1 | No mutation | No mutation |
| 11712_mo | No mutation | No mutation |
| 11712_fa | No mutation | No mutation |
| 11712_s1 | No mutation | No mutation |
| 11187_p1 | No mutation | No mutation |
| 11187_mo | No mutation | No mutation |
| 11187_fa | No mutation | No mutation |
| 11187_s1 | No mutation | No mutation |
| 11773_p1 | No mutation | No mutation |
| 11773_mo | No mutation | No mutation |
| 11773_fa | No mutation | No mutation |
| 11773_s1 | No mutation | No mutation |
| 11241_p1 | No mutation | No mutation |
| 11241_mo | No mutation | No mutation |
| 11241_fa | No mutation | No mutation |
| 11241_s1 | No mutation | No mutation |
| 11793_p1 | p.R488RH | No mutation |
| 11793_mo | p.R488RH | No mutation |
| 11793_fa | No mutation | No mutation |
| 11793_s1 | No mutation | No mutation |
| 11270_p1 | No mutation | No mutation |
| 11270_mo | No mutation | No mutation |
| 11270_fa | No mutation | No mutation |
| 11270_s1 | No mutation | No mutation |
| 11810_p1 | No mutation | No mutation |
| 11810_mo | No mutation | No mutation |
| 11810_fa | No mutation | No mutation |
| 11810_s1 | No mutation | No mutation |
| 11396_p1 | No mutation | No mutation |
| 11396_mo | No mutation | No mutation |
| 11396_fa | No mutation | No mutation |
| 11396_s1 | No mutation | No mutation |
| 11839_p1 | No mutation | No mutation |
| 11839_mo | No mutation | No mutation |
| 11839_fa | No mutation | No mutation |
| 11839_s1 | No mutation | No mutation |
| 11446_p1 | No mutation | No mutation |
| 11446_mo | No mutation | No mutation |
| 11446_fa | No mutation | No mutation |
| 11446_s1 | No mutation | No mutation |
| 11844_p1 | No mutation | No mutation |
| 11844_mo | No mutation | No DATA |
| 11844_fa | No mutation | No mutation |
| 11844_s1 | No mutation | No mutation |
| 11516_p1 | No mutation | No mutation |
| 11516_mo | No mutation | No mutation |
| 11516_fa | No mutation | No DATA |
| 11516_s1 | No mutation | No mutation |
| 11888_p1 | No mutation | No mutation |
| 11888_mo | No mutation | No mutation |
| 11888_fa | No mutation | No mutation |
| 11888_s1 | No mutation | No mutation |
| 11616_p1 | No mutation | No mutation |
| 11616_mo | No mutation | No mutation |
| 11616_fa | No mutation | No mutation |
| 11616_s1 | No mutation | No mutation |
| 11917_p1 | No mutation | No mutation |
| 11917_mo | No mutation | No mutation |
| 11917_fa | No mutation | No mutation |
| 11917_s1 | No mutation | No mutation |
| 11654_p1 | No mutation | No mutation |
| 11654_mo | No mutation | No mutation |
| 11654_fa | No mutation | No mutation |
| 11654_s1 | No mutation | No mutation |
| 11947_p1 | p.R488RH | No mutation |
| 11947_mo | p.R488RH | No mutation |
| 11947_fa | No mutation | No mutation |
| 11947_s1 | p.R488RH | No mutation |
| 12118_p1 | No mutation | No mutation |
| 12118_mo | No mutation | No mutation |
| 12118_fa | No mutation | No mutation |
| 12118_s1 | No mutation | No mutation |
| 12006_p1 | No mutation | No mutation |
| 12006_mo | No mutation | No mutation |
| 12006_fa | No mutation | No mutation |
| 12006_s1 | No mutation | No mutation |
| 12185_p1 | No mutation | No mutation |
| 12185_mo | No mutation | No mutation |
| 12185_fa | No mutation | No mutation |
| 12185_s1 | No mutation | No mutation |
| 12007_p1 | No mutation | No mutation |
| 12007_mo | No mutation | No mutation |
| 12007_fa | No mutation | No mutation |
| 12007_s1 | No mutation | No mutation |
| 12202_p1 | No mutation | No mutation |
| 12202_mo | No mutation | No mutation |
| 12202_fa | No mutation | No mutation |
| 12202_s1 | No mutation | No mutation |
| 12015_p1 | No mutation | No mutation |
| 12015_mo | No mutation | No mutation |
| 12015_fa | No mutation | No mutation |
| 12015_s1 | No mutation | No mutation |
| 12237_p1 | No mutation | No mutation |
| 12237_mo | No mutation | No mutation |
| 12237_fa | No mutation | No mutation |
| 12237_s1 | No mutation | No mutation |
| 12044_p1 | No mutation | No mutation |
| 12044_mo | No mutation | No mutation |
| 12044_fa | No mutation | No mutation |
| 12044_s1 | No mutation | No mutation |
| 12243_p1 | No mutation | No mutation |
| 12243_mo | No mutation | No mutation |
| 12243_fa | No mutation | No mutation |
| 12243_s1 | No mutation | No mutation |
| 12048_p1 | No mutation | No mutation |
| 12048_mo | No mutation | No mutation |
| 12048_fa | No mutation | No mutation |
| 12048_s1 | No mutation | No mutation |
| 12272_p1 | p.S362SL | No mutation |
| 12272_mo | No mutation | No mutation |
| 12272_fa | p.S362SL | No mutation |
| 12272_s1 | p.S362SL | No mutation |
| 12051_p1 | No mutation | No mutation |
| 12051_mo | No mutation | No mutation |
| 12051_fa | No mutation | No mutation |
| 12051_s1 | No mutation | No mutation |
| 12296_p1 | No mutation | No mutation |
| 12296_mo | No mutation | No mutation |
| 12296_fa | No mutation | No mutation |
| 12296_s1 | No mutation | No mutation |
| 12052_p1 | No mutation | No mutation |
| 12052_mo | No mutation | No mutation |
| 12052_fa | No mutation | No mutation |
| 12052_s1 | No mutation | No mutation |
| 12351_p1 | No mutation | No mutation |
| 12351_mo | No mutation | No mutation |
| 12351_fa | No mutation | No mutation |
| 12351_s1 | No mutation | No mutation |
| 12053_p1 | No mutation | No mutation |
| 12053_mo | No mutation | No mutation |
| 12053_fa | No mutation | No mutation |
| 12053_s1 | No mutation | No mutation |
| 12361_p1 | No mutation | No mutation |
| 12361_mo | No mutation | No mutation |
| 12361_fa | No mutation | No mutation |
| 12361_s1 | No mutation | No mutation |
| 12068_p1 | No mutation | No mutation |
| 12068_mo | No mutation | No mutation |
| 12068_fa | No mutation | No mutation |
| 12068_s1 | No mutation | No mutation |
| 12373_p1 | p.L144LF | No mutation |
| 12373_mo | No SEQUENCE | NO DATA |
| 12373_fa | No mutation | No mutation |
| 12373_s1 | p.L144LF | No mutation |
| 12088_p1 | No mutation | No mutation |
| 12088_mo | No mutation | No mutation |
| 12088_fa | No mutation | No mutation |
| 12088_s1 | No mutation | No mutation |
| 12388_p1 | No mutation | No mutation |
| 12388_mo | No mutation | No mutation |
| 12388_fa | No mutation | No mutation |
| 12388_s1 | No mutation | No mutation |
| 12100_p1 | No mutation | No mutation |
| 12100_mo | No mutation | No mutation |
| 12100_fa | No mutation | No mutation |
| 12100_s1 | No mutation | No mutation |
| 12492_p1 | No mutation | No mutation |
| 12492_mo | No mutation | No mutation |
| 12492_fa | No mutation | No mutation |
| 12492_s1 | No mutation | No mutation |
| 12850_p1 | No mutation | No mutation |
| 12850_mo | No mutation | No mutation |
| 12850_fa | No mutation | No mutation |
| 12850_s1 | No mutation | No mutation |
| 12494_p1 | No mutation | No mutation |
| 12494_mo | No mutation | No mutation |
| 12494_fa | p.R488RH | No mutation |
| 12494_s1 | No mutation | No mutation |
| 12924_p1 | No mutation | No mutation |
| 12924_mo | No mutation | No mutation |
| 12924_fa | No mutation | No mutation |
| 12924_s1 | No mutation | No mutation |
| 12510_p1 | p.G12GS | No mutation |
| 12510_mo | No mutation | No mutation |
| 12510_fa | No mutation | No mutation |
| 12510_s1 | No mutation | No mutation |
| 12964_p1 | No mutation | No mutation |
| 12964_mo | No mutation | No mutation |
| 12964_fa | No mutation | No mutation |
| 12964_s1 | No mutation | No mutation |
| 12521_p1 | No mutation | No mutation |
| 12521_mo | No mutation | No mutation |
| 12521_fa | No mutation | No mutation |
| 12521_s1 | No mutation | No mutation |
| 12977_p1 | No mutation | No mutation |
| 12977_mo | No mutation | No mutation |
| 12977_fa | No mutation | No mutation |
| 12977_s1 | No mutation | No mutation |
| 12552_p1 | No mutation | No mutation |
| 12552_mo | No mutation | No mutation |
| 12552_fa | No mutation | No mutation |
| 12552_s1 | No mutation | No mutation |
| 13076_p1 | No mutation | No mutation |
| 13076_mo | No mutation | No mutation |
| 13076_fa | No mutation | No mutation |
| 13076_s1 | No mutation | No mutation |
| 12603_p1 | No mutation | No mutation |
| 12603_mo | No mutation | No mutation |
| 12603_fa | No mutation | No mutation |
| 12603_s1 | No mutation | No mutation |
| 13082_p1 | No mutation | No mutation |
| 13082_mo | No mutation | No mutation |
| 13082_fa | No mutation | No mutation |
| 13082_s1 | No mutation | No mutation |
| 12644_p1 | No mutation | No mutation |
| 12644_mo | No mutation | No mutation |
| 12644_fa | No mutation | No mutation |
| 12644_s1 | No mutation | No mutation |
| 13092_p1 | No mutation | No mutation |
| 13092_mo | No mutation | No mutation |
| 13092_fa | No mutation | No mutation |
| 13092_s1 | No mutation | No mutation |
| 12650_p1 | No mutation | No mutation |
| 12650_mo | No mutation | No mutation |
| 12650_fa | No mutation | No mutation |
| 12650_s1 | No mutation | No mutation |
| 13133_p1 | No mutation | No mutation |
| 13133_mo | No mutation | No mutation |
| 13133_fa | No mutation | No mutation |
| 13133_s1 | No mutation | No mutation |
| 12656_p1 | No mutation | No mutation |
| 12656_mo | No mutation | No mutation |
| 12656_fa | No mutation | No mutation |
| 12656_s1 | No mutation | No mutation |
| 13152_p1 | No mutation | No mutation |
| 13152_mo | No mutation | No mutation |
| 13152_fa | No mutation | No mutation |
| 13152_s1 | No mutation | No mutation |
| 12749_p1 | No mutation | No mutation |
| 12749_mo | No mutation | No mutation |
| 12749_fa | No mutation | No mutation |
| 12749_s1 | No mutation | No mutation |
| 13165_p1 | No mutation | No mutation |
| 13165_mo | No mutation | No mutation |
| 13165_fa | No mutation | No mutation |
| 13165_s1 | No mutation | No mutation |
| 12786_p1 | No mutation | No mutation |
| 12786_mo | No mutation | No mutation |
| 12786_fa | No mutation | No mutation |
| 12786_s1 | No mutation | No mutation |
| 13188_p1 | No mutation | No mutation |
| 13188_mo | No mutation | No mutation |
| 13188_fa | No mutation | No mutation |
| 13188_s1 | No mutation | No mutation |
| 12824_p1 | No mutation | No mutation |
| 12824_mo | No mutation | No mutation |
| 12824_fa | No mutation | No mutation |
| 12824_s1 | No mutation | No mutation |
| 13215_p1 | No mutation | No mutation |
| 13215_mo | No mutation | No mutation |
| 13215_fa | No mutation | No mutation |
| 13215_s1 | No mutation | No mutation |
| 13528_p1 | No mutation | No mutation |
| 13528_mo | No mutation | No mutation |
| 13528_fa | No mutation | No mutation |
| 13528_s1 | No mutation | No mutation |
| 13222_p1 | No mutation | No mutation |
| 13222_mo | No mutation | No mutation |
| 13222_fa | No mutation | No mutation |
| 13222_s1 | No mutation | No mutation |
| 13544_p1 | No mutation | No mutation |
| 13544_mo | No mutation | No mutation |
| 13544_fa | No mutation | No mutation |
| 13544_s1 | No mutation | No mutation |
| 13272_p1 | No mutation | No mutation |
| 13272_mo | No mutation | No mutation |
| 13272_fa | No mutation | No mutation |
| 13272_s1 | No mutation | No mutation |
| 13557_p1 | No mutation | No mutation |
| 13557_mo | No mutation | No mutation |
| 13557_fa | No mutation | No mutation |
| 13557_s1 | No mutation | No mutation |
| 13287_p1 | No mutation | No mutation |
| 13287_mo | No mutation | No mutation |
| 13287_fa | No mutation | No mutation |
| 13287_s1 | No mutation | No mutation |
| 13634_p1 | No mutation | No mutation |
| 13634_mo | No mutation | No mutation |
| 13634_fa | No mutation | No mutation |
| 13634_s1 | No mutation | No mutation |
| 13307_p1 | No mutation | No mutation |
| 13307_mo | No mutation | No mutation |
| 13307_fa | No mutation | No mutation |
| 13307_s1 | No mutation | No mutation |
| 13642_p1 | No mutation | No mutation |
| 13642_mo | No mutation | No mutation |
| 13642_fa | No mutation | No mutation |
| 13642_s1 | No mutation | No mutation |
| 13309_p1 | No mutation | No mutation |
| 13309_mo | No mutation | No mutation |
| 13309_fa | No mutation | No mutation |
| 13309_s1 | No mutation | No mutation |
| 13682_p1 | No mutation | No mutation |
| 13682_mo | No mutation | No mutation |
| 13682_fa | No mutation | No mutation |
| 13682_s1 | No mutation | No mutation |
| 13335_p1 | No mutation | No mutation |
| 13335_mo | No mutation | No mutation |
| 13335_fa | No mutation | No mutation |
| 13335_s1 | No mutation | No mutation |
| 11099_p1 | No mutation | No mutation |
| 11099_mo | No mutation | No mutation |
| 11099_fa | No mutation | No mutation |
| 11099_s1 | No mutation | No mutation |
| 13358_p1 | No mutation | No mutation |
| 13358_mo | No mutation | No mutation |
| 13358_fa | No mutation | No mutation |
| 13358_s1 | No mutation | No mutation |
| 11132_p1 | No mutation | No mutation |
| 11132_mo | p.R488RH | No mutation |
| 11132_fa | No mutation | No mutation |
| 11132_s1 | p.R488RH | No mutation |
| 13389_p1 | No mutation | No mutation |
| 13389_mo | No mutation | No mutation |
| 13389_fa | No mutation | No mutation |
| 13389_s1 | No mutation | No mutation |
| 11224_p1 | No mutation | NO DATA |
| 11224_mo | No mutation | No mutation |
| 11224_fa | No mutation | No mutation |
| 11224_s1 | No mutation | No mutation |
| 13396_p1 | No mutation | No mutation |
| 13396_mo | No mutation | No mutation |
| 13396_fa | No mutation | No mutation |
| 13396_s1 | No mutation | No mutation |
| 11266_p1 | No mutation | No mutation |
| 11266_mo | No mutation | No mutation |
| 11266_fa | No mutation | No mutation |
| 11266_s1 | No mutation | No mutation |
| 13412_p1 | No mutation | No mutation |
| 13412_mo | No mutation | No mutation |
| 13412_fa | No mutation | No mutation |
| 13412_s1 | No mutation | No mutation |
| 11398_p1 | No mutation | No mutation |
| 11398_mo | No mutation | No mutation |
| 11398_fa | No mutation | No mutation |
| 11398_s1 | No mutation | No mutation |
| 13493_p1 | No mutation | No mutation |
| 13493_mo | No mutation | No mutation |
| 13493_fa | No mutation | No mutation |
| 13493_s1 | No mutation | No mutation |
| 13456_p1 | No mutation | No mutation |
| 11810_mo | No mutation | No mutation |

Results for SLC22A5 exon sequencing and MLPA
P1, proband (affected);
mo, mother;
fa, father;
s1, sibling (unaffected)

### Example 8

### Diagnostic Metabolite Abnormalities in Plasma and Urine

Two affected brothers from AGRE family AU 0177 had a normal facial appearance in childhood and were otherwise non-dysmorphic. They both had normal plasma free carnitine levels (33 and 34 μmol/l, normal 22-65) at recent ages of 15 and 17 years. In urine of the affected brothers, HTML and γBB were undetectable, and the excretion of TML was 3-fold that of controls (FIG. 4). Plasma from the two brothers and from five SSC probands showed a significant increase in TML, complete absence of HTML, and severely reduced levels of γBB except for one case (FIG. 6A). The (HTML+γBB)/TML ratio was very low in patients as compared to control plasma and may be an excellent index of TMLD activity (FIG. 6B). These data indicate that TMLHE deficiency represents a novel inborn error of carnitine biosynthesis. To search for other evidence of a common TMLHE deficiency and for other defects in carnitine biosynthesis, urine from 29 SSC probands that did not have any known TMLHE mutations was studied and did not reveal any abnormal carnitine metabolites.

### Example 9

### Results of Screening 353 Probands from SSC for Plasma Carnitine Metabolites

As part of the neuronal carnitine deficiency hypothesis for autism, the inventors considered that low dietary intake of carnitine results in low plasma carnitine. In order to assess plasma carnitine levels in autism, the inventors reviewed 364 plasma samples from the Simons Simplex Collection (SSC) including from the 297 youngest male probands, from the 56 youngest female probands, and from all 11 siblings where plasma was available. Additional control samples from other sources were also available. The inventors contemplated that if dietary deficiency of carnitine was a factor in non-dysmorphic autism and reports of low plasma free carnitine in autism were accurate, that young patients should be particularly considered, because probands might have a higher meat intake as they became older. Measurements of free carnitine levels and carnitine biosynthesis intermediates were taken in these samples, and samples of interest are shown in Table S9.

TABLE S9
Carnitine biosynthesis intermediates in plasma from SSC probands.

| | | | | Ratio | Ratio | |
|---|---|---|---|---|---|---|
| | | | | area | conc | |
| | | | | hTML/ | [gBB]/ | |
| | TML | gBB | carnitine | TML | [TML] | Mutation |
| Control | 0.2-1.2 | 0.3-1.4 | 22.3-54.8 | 0.1-1.4 | 0.6-5.9 | |
| range | | | | | | |
| | Three autism male probands with normal results | | | | | |
| 13963.p1 | 0.37 | 0.45 | 38.0 | 0.38 | 1.22 | N/A |
| male | | | | | | |
| 13978.p1 | 0.26 | 0.33 | 29.5 | 0.93 | 1.28 | N/A |
| male | | | | | | |
| 14010.p1 | 0.45 | 0.49 | 39.0 | 0.37 | 1.07 | N/A |
| male | | | | | | |
| | Four males with possible TMLHE deficiency | | | | | |
| 13793.p1 | 1.34 | 0.51 | 36.5 | 0.11 | 0.38 | R70H |
| male | | | | | | |
| 12055.p1 | 2.91 | 0.40 | 35.63 | 0.06 | 0.14 | Unknown |
| male | | | | | | |
| 12971.p1 | 1.91 | 0.10 | 27.66 | 0.01 | 0.05 | Exon |
| male | | | | | | deletion |
| 14243.p1 | 1.91 | 0.04 | 15.77 | 0.001 | 0.02 | Pending |
| | One female with high gBB | | | | | |
| 11266.p1 | 0.42 | 5.70 | 56.2 | 0.69 | 13.61 | Untested |
| female | | | | | | |

There were no detectable differences between any of the autism subgroups for plasma free carnitine levels, and no differences between autism and control samples. The male probands ranged in age from 48 to 62 months of age. In particular embodiments of the invention, dietary carnitine deficiency occurs starting with the introduction of solid foods until generous meat intake is established. Thus in certain embodiments the highest risk for dietary deficiency is from approximately 6 to 24 months of age. Thus the available data does not address the consideration of dietary carnitine deficiency in this age range, but they do indicate that low plasma carnitine is not common in autism probands from age 48 months onward, thus failing to confirm reports of low plasma carnitine in autism (Filipek et al., 2004).

The inventors considered males providing samples for plasma carnitine metabolites, and the biochemical data strongly or moderately suggested TMLHE deficiency in addition to those tested in other patients found earlier (Table S9). One of these was found to have an R70H missense mutation that likely has a hypomorphic effect, one was found to have an exonic deletion, a third had no mutation on exon sequencing but may have a mutation yet to be identified, and a fourth is yet to be sequenced. Enzyme assays for TMLHE activity on lymphoblasts from these cases are performed. In addition, a female proband with 10-fold elevated levels of γ-butyrobetaine (gBB) in plasma (Table S9) was identified. In some embodiments, this represents examples of deficiency of BBOXI or another enzyme or transporter in carnitine metabolism and leads to the discovery of additional novel inborn error of carnitine biosynthesis. Performing plasma analysis for carnitine metabolites in all samples from SSC probands identifies other individuals with abnormalities of carnitine metabolism.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.

### PATENTS

U.S. Pat. No. 4,683,195
U.S. Pat. No. 4,883,750
U.S. Pat. No. 4,946,773
U.S. Pat. No. 5,279,721
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,483
U.S. Pat. No. 5,849,487
U.S. Pat. No. 5,849,497
U.S. Pat. No. 5,849,546
U.S. Pat. No. 5,849,547
U.S. Pat. No. 5,851,770
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,337
U.S. Pat. No. 5,866,366
U.S. Pat. No. 5,882,864
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,630
U.S. Pat. No. 5,922,574
U.S. Pat. No. 5,925,517
U.S. Pat. No. 5,925,525
U.S. Pat. No. 5,928,862
U.S. Pat. No. 5,928,869
U.S. Pat. No. 5,928,870
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 4,683,202
U.S. Pat. No. 4,800,159
U.S. Pat. No. 5,849,486
U.S. Pat. No. 5,851,772
U.S. Pat. No. 5,900,481
U.S. Pat. No. 5,919,626

### PUBLICATIONS

Abecasis, G. R., Chemy, S. S., Cookson, W. O., and Cardon, L. R. (2002). Merlin—rapid analysis of dense genetic maps using sparse gene flow trees. Nat. Genet. 30, 97-101.
Bauman, M. L. and Kemper, T. L. (2005). Neuroanatomic observations of the brain in autism: a review and future directions. Int. J. Dev. Neurosci. 23, 183-187.
Boone, P. M., Liu, P., Zhang, F., Carvalho, C. M., Towne, C. F., Batish, S. D., and Lupski, J. R. (2011). Alu-specific microhomology-mediated deletion of the final exon of SPAST in three unrelated subjects with hereditary spastic paraplegia. Genet. Med. 13, 582-592.
Bradford, M. M. (1976). A rapid and sensitive method for the quantitation of microgram quantities of protein utilizing the principle of protein-dye binding. Anal. Biochem. 72, 248-254.
Brambilla, P., Perez, J., Barale, F., Schettini, G., and Soares, J. C. (2003). GABAergic dysfunction in mood disorders. Mol. Psychiatry. 8, 721-37, 715.
Carrel, L. and Willard, H. F. (2005). X-inactivation profile reveals extensive variability in X-linked gene expression in females. Nature 434, 400-404.
Chao, H. T., Chen, H., Samaco, R. C., Xue, M., Chahrour, M., Yoo, J., Neul, J. L., Gong, S., Lu, H. C., Heintz, N., Ekker, M., Rubenstein, J. L., Noebels, J. L., Rosenmund, C., and Zoghbi, H. Y. (2010). Dysfunction in GABA signalling mediates autism-like stereotypies and Rett syndrome phenotypes. Nature 468, 263-269.
Conference Proceedings (2004). Carnitine. The science behind a conditionally essential nutrient. Proceedings of a conference. Mar. 25-26, 2004. Bethesda, Md., USA. Ann. N.Y. Acad. Sci. 1033, ix-xi.
Craddock, N., Hurles, M. E., Cardin, N., Pearson, R. D., Plagnol, V., Robson, S., Vukcevic, D., Barnes, C., Conrad, D. F., Giannoulatou, E., Holmes, C., Marchini, J. L., Stirrups, K., Tobin, M. D., Wain, L. V., Yau, C., Aerts, J., Ahmad, T., Andrews, T. D., Arbury, H., Attwood, A., Auton, A., Ball, S. G., Balmforth, A. J., Barrett, J. C., Barroso, I., Barton, A., Bennett, A. J., Bhaskar, S., Blaszczyk, K., Bowes, J., Brand, O. J., Braund, P. S., Bredin, F., Breen, G., Brown, M. J., Bruce, I. N., Bull, J., Burren, O. S., Burton, J., Byrnes, J., Caesar, S., Clee, C. M., Coffey, A. J., Connell, J. M., Cooper, J. D., Dominiczak, A. F., Downes, K., Drummond, H. E., Dudakia, D., Dunham, A., Ebbs, B., Eccles, D., Edkins, S., Edwards, C., Elliot, A., Emery, P., Evans, D. M., Evans, G., Eyre, S., Farmer, A., Ferrier, I. N., Feuk, L., Fitzgerald, T., Flynn, E., Forbes, A., Forty, L., Franklyn, J. A., Freathy, R. M., Gibbs, P., Gilbert, P., Gokumen, O., Gordon-Smith, K., Gray, E., Green, E., Groves, C. J., Grozeva, D., Gwilliam, R., Hall, A., Hammond, N., Hardy, M., Harrison, P., Hassanali, N., Hebaishi, H., Hines, S., Hinks, A., Hitman, G. A., Hocking, L., Howard, E., Howard, P., Howson, J. M., Hughes, D., Hunt, S., Isaacs, J. D., Jain, M., Jewell, D. P., Johnson, T., Jolley, J. D., Jones, I. R., Jones, L. A., Kirov, G., Langford, C. F., Lango-Allen, H., Lathrop, G. M., Lee, J., Lee, K. L., Lees, C., Lewis, K., Lindgren, C. M., Maisuria-Armer, M., Maller, J., Mansfield, J., Martin, P., Massey, D. C., McArdle, W. L., McGuffin, P., McLay, K. E., Mentzer, A., Mimmack, M. L., Morgan, A. E., Morris, A. P., Mowat, C., Myers, S., Newman, W., Nimmo, E. R., O'Donovan, M. C., Onipinla, A., Onyiah, I., Ovington, N. R., Owen, M. J., Palin, K., Parnell, K., Pernet, D., Perry, J. R., Phillips, A., Pinto, D., Prescott, N. J., Prokopenko, I., Quail, M. A., Rafelt, S., Rayner, N. W., Redon, R., Reid, D. M., Renwick, Ring, S. M., Robertson, N., Russell, E., St, C. D., Sambrook, J. G., Sanderson, J. D., Schuilenburg, H., Scott, C. E., Scott, R., Seal, S., Shaw-Hawkins, S., Shields, B. M., Simmonds, M. J., Smyth, D. J., Somaskantharajah, E., Spanova, K., Steer, S., Stephens, J., Stevens, H. E., Stone, M. A., Su, Z., Symmons, D. P., Thompson, J. R., Thomson, W., Travers, M. E., Turnbull, C., Valsesia, A., Walker, M., Walker, N. M., Wallace, C., Warren-Perry, M., Watkins, N. A., Webster, J, Weedon, M. N., Wilson, A. G., Woodburn, M., Wordsworth, B. P., Young, A. H., Zeggini, E., Carter, N. P., Frayling, T. M., Lee, C., McVean, G., Munroe, P. B., Palotie, A., Sawcer, S. J., Scherer, S. W., Strachan, D. P., Tyler-Smith, C., Brown, M. A., Burton, P. R., Caulfield, M. J., Comp ston, A., Farrall, M., Gough, S. C., Hall, A. S., Hattersley, A. T., Hill, A. V., Mathew, C. G., Pembrey, M., Satsangi, J., Stratton, M. R., Worthington, J., Deloukas, P., Duncanson, A., Kwiatkowski, D. P., McCarthy, M. I., Ouwehand, W., Parkes, M., Rahman, N., Todd, J. A., Samani, N. J., and Donnelly, P. (2010). Genome-wide association study of CNVs in 16,000 cases of eight common diseases and 3,000 shared controls. Nature 464, 713-720.
Filipek, P. A., Juranek, J., Nguyen, M. T., Cummings, C., and Gargus, J. J. (2004). Relative carnitine deficiency in autism. J Autism Dev. Disord. 34, 615-623.
Fischbach, G. D. and Lord, C. (2010). The Simons Simplex Collection: a resource for identification of autism genetic risk factors. Neuron 68, 192-195.
Flanagan, J. L., Simmons, P. A., Vehige, J., Willcox, M. D., and Garrett, Q. (2010). Role of carnitine in disease. Nutr. Metab (Lond) 7, 30.
Fujita, M., Nakanishi, T., Shibue, Y., Kobayashi, D., Moseley, R. H., Shirasaka, Y., and Tamai, I. (2009). Hepatic uptake of gamma-butyrobetaine, a precursor of carnitine biosynthesis, in rats. Am. J. Physiol Gastrointest. Liver Physiol 297, G681-G686.
Gargus, J. J. and Imtiaz, F. (2008). Mitochondrial Energy-Deficient Endophenotype in Autism. American Journal of Biochemistry and Biotechnology 4, 198-207.
Geier, D. A., Kern, J. K., Davis, G., King, P. G., Adams, J. B., Young, J. L., and Geier, M. R. (2011). A prospective double-blind, randomized clinical trial of levocarnitine to treat autism spectrum disorders. Med. Sci. Monit. 17, 115-123.
Girirajan, S., Ro senfeld, J. A., Cooper, G. M., Antonacci, F., Siswara, P., Itsara, A., Vives, L., Walsh, T., McCarthy, S. E., Baker, C., Mefford, H. C., Kidd, J. M., Browning, S. R., Browning, B. L., Dickel, D. E., Levy, D. L., Ballif, B. C., Platky, K., Farber, D. M., Gowans, G. C., Wetherbee, J. J., Asamoah, A., Weaver, D. D., Mark, P. R., Dickerson, J., Garg, B. P., Ellingwood, S. A., Smith, R., Banks, V. C., Smith, W., McDonald, M. T., Hoo, J. J., French, B. N., Hudson, C., Johnson, J. P., Ozmore, J. R., Moeschler, J. B., Surti, U., Escobar, L. F., El-Khechen, D., Gorski, J. L., Kussmann, J., Salbert, B., Lacassie, Y., Biser, A., McDonald-McGinn, D. M., Zackai, E. H., Deardorff, M. A., Shaikh, T. H., Haan, E., Friend, K. L., Fichera, M., Romano, C., Gecz, J., DeLisi, L. E., Sebat, J., King, M. C., Shaffer, L. G., and Eichler, E. E. (2010). A recurrent 16p12.1 microdeletion supports a two-hit model for severe developmental delay. Nat. Genet. 42, 203-209.
Innis et al., “DNA sequencing with Thermus aquaticus DNA polymerase and direct sequencing of polymerase chain reaction-amplified DNA,” Proc Natl Acad Sci USA. 85(24):9436-9440, 1988.
Jacquemont, M.-L., Sanlaville, D., Redon, R., Raoul, O., Cormier-Daire, V., Lyonnet, S., Amiel, J., Le, M. M., Heron, D., De Blois, M.-C., Prieur, M., Vekemans, M., Carter, N. P., Munnich, A., Colleaux, L., and Phillipe, A. (2006). Array-based comparative genomic hybridization identifies high frequency of cryptic chromosomal rearrangements in patients with syndromic autism spectrum disorders. J. Med. Genet. 43, 843-849.
Kalra, V., Seth, R., and Sapra, S. (2005). Autism—experiences in a tertiary care hospital. Indian J. Pediatr. 72, 227-230.
Kim, Y. S., Leventhal, B. L., Koh, Y. J., Fombonne, E., Laska, E., Lim, E. C., Cheon, K. A., Kim, S. J., Kim, Y. K., Lee, H., Song, D. H., and Grinker, R. R. (2011). Prevalence of Autism Spectrum Disorders in a Total Population Sample. Am. J. Psychiatry. May 9. [Epub ahead of print]
Koizumi, A., Nozaki, J., Ohura, T., Kayo, T., Wada, Y., Nezu, J., Ohashi, R., Tamai, I., Shoji, Y., Takada, G., Kibira, S., Matsuishi, T., and Tsuji, A. (1999). Genetic epidemiology of the carnitine transporter OCTN2 gene in a Japanese population and phenotypic characterization in Japanese pedigrees with primary systemic carnitine deficiency. Hum. Mol. Genet. 8, 2247-2254.
Kong, A. and Cox, N. J. (1997). Allele-sharing models: LOD scores and accurate linkage tests. Am. J. Hum. Genet. 61, 1179-1188.
Kwoh et al., “Transcription-based amplification system and detection of amplified human immunodeficiency virus type 1 with a bead-based sandwich hybridization format, Proc Natl Acad Sci USA. 86(4):1173-1177, 1989.
Lahjouji, K., Elimrani, I., Wu, J., Mitchell, G. A., and Qureshi, I. A. (2002). A heterozygote phenotype is present in the jvs+/−mutant mouse livers. Mol. Genet. Metab 76, 76-80.
Lombard, J. (1998). Autism: a mitochondrial disorder? Med. Hypotheses 50, 497-500.
Longo, N., Amat di San Filippo, C., and Pasquali, M. (2006). Disorders of carnitine transport and the carnitine cycle. Am. J. Med. Genet. C. Semin. Med. Genet. 142C, 77-85.
Mandal, R., Guo, A. C., Chaudhary, K. K., Liu, P., Yallou, F. S., Dong, E., Aziat, F., and D. S. Wishart. (2012). Multi-platform characterization fo the human cerebrospinal fulid metabolome: a comprehensive and quantitative update. Gen. Med. 4:38.
Marshall, C. R., Noor, A., Vincent, J. B., Lionel, A. C., Feuk, L., Skaug, J., Shago, M., Moessner, R., Pinto, D., Ren, Y., Thiruvahindrapduram, B., Fiebig, A., Schreiber, S., Friedman, J., Ketelaars, C. E., Vos, Y. J., Ficicioglu, C., Kirkpatrick, S, Nicolson, R., Sloman, L., Summers, A., Gibbons, C. A., Teebi, A., Chitayat, D., Weksberg, R., Thompson, A., Vardy, C., Crosbie, V., Luscombe, S., Baatjes, R., Zwaigenbaum, L., Roberts, W., Fernandez, B., Szatmari, P., and Scherer, S. W. (2008). Structural variation of chromosomes in autism spectrum disorder. Am J. Hum. Genet. 82, 477-488.
Matise, T. C., Chen, F., Chen, W., De La Vega, F. M., Hansen, M., He, C., Hyland, F. C., Kennedy, G. C., Kong, X., Murray, S. S., Ziegle, J. S., Stewart, W. C., and Buyske, S. (2007). A second-generation combined linkage physical map of the human genome. Genome Res. 17, 1783-1786.
McCarroll, S. A., Hadnott, T. N., Perry, G. H., Sabeti, P. C., Zody, M. C., Barrett, J. C., Dallaire, S., Gabriel, S. B., Lee, C., Daly, M. J., and Altshuler, D. M. (2006). Common deletion polymorphisms in the human genome. Nat. Genet. 38, 86-92.
Miles, J. H., Takahashi, T. N., Hong, J., Munden, N., Flournoy, N., Braddock, S. R., Martin, R. A., Bocian, M. E., Spence, M. A., Hillman, R. E., and Farmer, J. E. (2008). Development and validation of a measure of dysmorphology: useful for autism subgroup classification. Am. J. Med. Genet. A 146A, 1101-1116.
Monfregola, J., Napolitano, G., Conte, I., Cevenini, A., Migliaccio, C., D'Urso, M., and Ursini, M. V. (2007). Functional characterization of the TMLH gene: promoter analysis, in situ hybridization, identification and mapping of alternative splicing variants. Gene 395, 86-97.
Mostafa, G. A., El-Gamal, H. A., El-Wakkad, A. S. E., El-Shorbagy, O. E., and Hamza, M. M. (2005). Polyunsaturated Fatty Acids, Carnitine and Lactate as Biological Markers of Brain Energy in Autistic Children. International Journal of Child Neuropsychiatry 2, 179-188.
O'Connell, J. R. and Weeks, D. E. (1998). PedCheck: a program for identification of genotype incompatibilities in linkage analysis. Am. J. Hum. Genet. 63, 259-266.
Ohara et al., “One-sided polymerase chain reaction: the amplification of cDNA,”
Okanari, K., Takahashi, M., Maeda, T., Sato, K., Suenobu, S., and Izumi, T. (2007). Hypocarnitinemic hypoglycemia and heart failure in an infant with a constant parenteral elementary nutrition during measles vaccination-related febrile illness. Neuropediatrics 38, 317-319.
Ou, Z., Kang, S. H., Shaw, C. A., Carmack, C. E., White, L. D., Patel, A., Beaudet, A. L., Cheung, S. W., and Chinault, A. C. (2008). Bacterial artificial chromosome-emulation oligonucleotide arrays for targeted clinical array-comparative genomic hybridization analyses. Genet Med 10, 278-289.
Rossignol, D. A. and Frye, R. E. (2011). Mitochondrial dysfunction in autism spectrum disorders: a systematic review and meta-analysis. Mol. Psychiatry.
Sambrook et al., In: Molecular Cloning: A Laboratory Manual, Vol. 1, Cold Spring Harbor Laboratory Press, Cold Spring Harbor, N.Y., Ch. 7, 7.19 17.29, 1989.
Sanders, A. R., Levinson, D. F., Duan, J., Dennis, J. M., Li, R., Kendler, K. S., Rice, J. P., Shi, J., Mowry, B. J., Amin, F., Silverman, J. M., Buccola, N. G., Byerley, W. F., Black, D. W., Freedman, R., Cloninger, C. R., and Gejman, P. V. (2010). The Internet-based MGS2 control sample: self report of mental illness. Am. J. Psychiatry 167, 854-865.
Sanders, S. J., Ercan-Sencicek, A. G., Hus, V., Luo, R., Murtha, M. T., Moreno-De-Luca, D., Chu, S. H., Moreau, M. P., Gupta, A. R., Thomson, S. A., Mason, C. E., Bilguvar, K., Celestino-Soper, P. B. S., Choi, M., Crawford, E. L., Davis, L., Davis Wright, N. R., Dhodapkar, R. M., DiCola, M., DiLullo, N. M., Fernandez, T. V., Fielding-Singh, V., Fishman, D. O., Frahm, S., Garagaloyan R., Goh, G. S., Kammela, S., Klei, L., Lowe, J. K., Lund, S. C., McGrew, A. D., Meyer, K. A., Moffat, W. J., Murdoch, J. D., O'Roak, B. J., Ober, G. T., Pottenger, R. S., Raubeson, M. J., Song, Y., Wang, Q., Yaspan, B. L., Yu, T. W., Yurkiewicz, I. R., Beaudet, A. L., Cantor R. M., Curland, M., Grice, D. E., Gunel, M., Lifton, R. P., Mane, S. M., Martin, D. M., Shaw, C. A., Sheldon, M., Tischfield, J. A., Walsh, C. A., Morrow, E. M., Ledbetter, D. H., Fombonne, E., Lord, C., Martin, C. L., Brooks, A. I., Sutcliffe, J. S., Cook, Jr. E. H., Geschwind, D., Roeder, K., Devlin, B., and State, M. W. Multiple recurrent de novo copy number variations (CNVs), including duplications of the 7q11.23 Williams-Beuren syndrome region, are strongly associated with autism. Neuron 70, 863-885.
Scott, F. J., Baron-Cohen, S., Bolton, P., and Brayne, C. (2002). Brief report: prevalence of autism spectrum conditions in children aged 5-11 years in Cambridgeshire, UK. Autism 6, 231-237.
Sebat, J., Lakshmi, B., Malhotra, D., Troge, J., Lese-Martin, C., Walsh, T., Yamrom, B., Yamrom, B., Yoon, S., Krasnitz, A., Kendall, J., Leotta, A., Pai, D., Zhang, R., Lee, Y. H., Hicks, J., Spence, S. J., Lee, A. T., Puura, K., Lehtimaki, T., Ledbetter, D., Gregersen, P. K., Bregman, J., Sutcliffe, J. S., Jobanputra, V., Chung, W., Warburton, D., King, M. C., Skuse, D., Geschwind, D. H., Gilliam, T. C., Ye, K., and Wigler, M. (2007). Strong association of de novo copy number mutations with autism. Science 316, 445-449.
Sebat, J., Levy, D. L., and McCarthy, S. E. (2009). Rare structural variants in schizophrenia: one disorder, multiple mutations; one mutation, multiple disorders. Trends in Genetics 25, 528-535.
Shinawi, M., Schaaf, C. P., Bhatt, S. S., Xia, Z., Patel, A., Cheung, S. W., Lanpher, B., Nagl, S., Herding, H. S., Nevinny-Stickel, C., Immken, L. L., Patel, G. S., German, J. R., Beaudet, A. L., and Stankiewicz, P. (2009). A small recurrent deletion within 15q13.3 is associated with a range of neurodevelopmental phenotypes. Nat Genet. 41, 1269-1271.
Slonim, A. E., Borum, P. R., Tanaka, K., Stanley, C. A., Kasselberg, A. G., Greene, H. L., and Burr, I. M. (1981). Dietary-dependent carnitine deficiency as a cause of nonketotic hypoglycemia in an infant. J. Pediatr. 99, 551-555.
Strijbis, K., Vaz, F. M., and Distel, B. (2010). Enzymology of the carnitine biosynthesis pathway. IUBMB. Life 62, 357-362.
Swiegers, J. H., Vaz, F. M., Pretorius, I. S., Wanders, R. J, and Bauer, F. F. (2002). Carnitine biosynthesis in Neurospora crassa: identification of a cDNA coding for epsilon-N-trimethyllysine hydroxylase and its functional expression in Saccharomyces cerevisiae. FEMS Microbiol. Lett. 210, 19-23.
van Bon, B. W., Mefford, H. C., Menten, B., Koolen, D. A., Sharp, A. J., Nillesen, W. M., Innis, J. W., de Ravel, T. J., Mercer, C. L., Fichera, M., Stewart, H., Connell, L. E., Ounap, K., Lachlan, K., Castle, B., Van der, A. N., van, R. C., Nobrega, M. A., Serra-Juhe, C., Simonic, I., de, L. N., Pfundt, R., Bongers, E. M., Baker, C., Finnemore, P., Huang, S., Maloney, V. K., Crolla, J. A., van, K. M., Elia, M., Vandeweyer, G., Fryns, J. P., Janssens, S., Foulds, N., Reitano, S., Smith, K., Parkel, S., Loeys, B., Woods, C. G., Oostra, A., Speleman, F., Pereira, A. C., Kurg, A., Willatt, L., Knight, S. J., Vermeesch, J. R., Romano, C., Barber, J. C., Mortier, G., Perez-Jurado, L. A., Kooy, F., Brunner, H. G., Eichler, E. E., Kleefstra, T., and de Vries, B. B. (2009). Further delineation of the 15q13 microdeletion and duplication syndromes: A clinical spectrum varying from non-pathogenic to a severe outcome. J Med Genet. 46, 511-523.
Vaz, F. M., Melegh, B., Bene, J., Cuebas, D., Gage, D. A., Bootsma, A., Vreken, P., van Gennip, A. H., Bieber, L. L., and Wanders, R. J. (2002). Analysis of carnitine biosynthesis metabolites in urine by HPLC-electrospray tandem mass spectrometry. Clin. Chem. 48, 826-834.
Vaz, F. M., Ofman, R., Westinga, K., Back, J. W., and Wanders, R. J. (2001). Molecular and Biochemical Characterization of Rat epsilon —N-Trimethyllysine Hydroxylase, the First Enzyme of Carnitine Biosynthesis. J. Biol. Chem. 276, 33512-33517.
Vaz, F. M. and Wanders, R. J. (2002). Carnitine biosynthesis in mammals. Biochem. J. 361, 417-429.
Whittemore, A. S. and Halpern, J. (1994). A class of tests for linkage using affected pedigree members. Biometrics 50, 118-127.
Wong, V. C. and Hui, S. L. (2008). Epidemiological study of autism spectrum disorder in China. J Child Neurol. 23, 67-72.
Worthley, L. I., Fishlock, R. C., and Snoswell, A. M. (1983). Carnitine deficiency with hyperbilirubinemia, generalized skeletal muscle weakness and reactive hypoglycemia in a patient on long-term total parenteral nutrition: treatment with intravenous L-carnitine. JPEN J. Parenter. Enteral Nutr. 7, 176-180.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of treating an individual having autism or at risk of developing autism or autism regression and in need of determination of need of carnitine supplementation, comprising the step of determining the level of one or more carnitine metabolites in a sample from the individual and when the level of the one or more carnitine metabolites is abnormal the individual is provided with an effective amount of a carnitine metabolite.

2. The method of claim 1, wherein the individual is an asymptomatic infant at risk of developing autism.

3. The method of claim 1, wherein the individual has non-dysmorphic autism.

4. The method of claim 1, further comprising the step of assaying for a mutation in a carnitine-related gene.

5. The method of claim 4, wherein the gene is TMLHE, SCL22A5, SLC6A14, SLC25A45, SHMT1, SHMT2, SLC6A13, HTMLA, ALDH9A1, TMABA-DH, BBOX1, CPT1-C, CPT2, SLC25A20, or a combination thereof.

6. The method of claim 1, wherein the sample is cerebrospinal fluid, plasma, or urine.

7. The method of claim 1, wherein the ratio of one carnitine metabolite to another carnitine metabolite is determined.

8. The method of claim 1, further defined as determining the plasma to CSF ratio of one or more carnitine metabolites.

9. The method of claim 1, wherein the individual has at least one sibling that has autism or has abnormal carnitine levels in the CSF, plasma, or urine or has a defect in a carnitine-related gene.

10. The method of claim 9, wherein the sibling is a male.

11. A method of achieving a desired level of carnitine in an individual in need thereof, said method comprising the steps of:
determining a level of at least one carnitine metabolite from a sample from an individual that has autism or is at risk of developing autism and that has a defect in a carnitine-related gene; and
providing an effective amount of a carnitine metabolite to the individual

12. The method of claim 11, wherein the individual has non-dysmorphic autism.

13. The method of claim 11, wherein the carnitine-related gene is TMLHE, SCL22A5, SLC6A14, SLC25A45, SHMT1, SHMT2, SLC6A13, HTMLA, ALDH9A1, TMABA-DH, BBOX1, CPT1-C, CPT2, SLC25A20, or a combination thereof.

14. The method of claim 11, wherein the sample is cerebrospinal fluid, plasma, or urine.

15. The method of claim 11, wherein the ratio of one carnitine metabolite to another carnitine metabolite is determined.

16. The method of claim 11, further defined as determining the plasma to CSF ratio of one or more carnitine metabolites.

17. The method of claim 11, wherein the individual has at least one sibling that has autism or has abnormal carnitine levels in the CSF, plasma, or urine or has a defect in a carnitine-related gene.


SEQUENCE LISTING (85 sequences)

SEQ ID NO: 1 - DNA, 31 bp, Artificial Sequence
  Synthetic primer
ctacagaaacagagagatggcagtatcacag

SEQ ID NO: 2 - DNA, 32 bp, Artificial Sequence
  Synthetic primer
gtggtgattcctcaaggatctacaactagaat

SEQ ID NO: 3 - DNA, 30 bp, Artificial Sequence
  Synthetic primer
tttctcctaatgctatccctcccctagacc

SEQ ID NO: 4 - DNA, 32 bp, Artificial Sequence
  Synthetic primer
gatgtgcaagacctctatacttgctattgtca

SEQ ID NO: 5 - DNA, 31 bp, Artificial Sequence
  Synthetic primer
gtcttgagctttacaggagagaagcagaatg

SEQ ID NO: 6 - DNA, 32 bp, Artificial Sequence; identical to SEQ ID NO: 4
  Synthetic primer

SEQ ID NO: 7 - DNA, 30 bp, Artificial Sequence
  Synthetic primer
ctccagaaagcacccctacagacatagaga

SEQ ID NO: 8 - DNA, 30 bp, Artificial Sequence
  Synthetic primer
ctctcatcttctagagactgcctgccttac

SEQ ID NO: 9 - DNA, 30 bp, Artificial Sequence; identical to SEQ ID NO: 3
  Synthetic primer

SEQ ID NO: 10 - DNA, 32 bp, Artificial Sequence
  Synthetic primer
gcacttgagggaggaagataaatggagttaac

SEQ ID NO: 11 - DNA, 37 bp, Artificial Sequence
  Synthetic primer
gtaaaacgacggccagtaacagaagctcctccagtgc

SEQ ID NO: 12 - DNA, 37 bp, Artificial Sequence
  Synthetic primer
caggaaacagctatgaccgccagggttaaggtgagta

SEQ ID NO: 13 - DNA, 41 bp, Artificial Sequence
  Synthetic primer
gtaaaacgacggccagtcaatttctcctttgtttaatcctc

SEQ ID NO: 14 - DNA, 38 bp, Artificial Sequence
  Synthetic primer
caggaaacagctatgacggtcctgtcaactccacaaaa

SEQ ID NO: 15 - DNA, 37 bp, Artificial Sequence
  Synthetic primer
gtaaaacgacggccagtcccctcaacccttattgctt

SEQ ID NO: 16 - DNA, 37 bp, Artificial Sequence
  Synthetic primer
caggaaacagctatgaccttgccatgttccctgtctt

SEQ ID NO: 17 - DNA, 37 bp, Artificial Sequence
  Synthetic primer
gtaaaacgacggccagttcatgcataccactcaaacc

SEQ ID NO: 18 - DNA, 37 bp, Artificial Sequence
  Synthetic primer
caggaaacagctatgaccgagggtgaccatacctgtt

SEQ ID NO: 19 - DNA, 37 bp, Artificial Sequence
  Synthetic primer
gtaaaacgacggccagttccacagttttctccccaac

SEQ ID NO: 20 - DNA, 37 bp, Artificial Sequence
  Synthetic primer
caggaaacagctatgacctccccagaagaagatgctg

SEQ ID NO: 21 - DNA, 36 bp, Artificial Sequence
  Synthetic primer
gtaaaacgacggccagtcagggcccagcacatataa

SEQ ID NO: 22 - DNA, 38 bp, Artificial Sequence
  Synthetic primer
caggaaacagctatgacttctttggttggtggctattt

SEQ ID NO: 23 - DNA, 37 bp, Artificial Sequence
  Synthetic primer
gtaaaacgacggccagtacctcagcttggtgaggaga

SEQ ID NO: 24 - DNA, 37 bp, Artificial Sequence
  Synthetic primer
caggaaacagctatgacgatgaggcttgcttccagag

SEQ ID NO: 25 - DNA, 37 bp, Artificial Sequence; identical to SEQ ID NO: 11
  Synthetic primer

SEQ ID NO: 26 - DNA, 37 bp, Artificial Sequence
  Synthetic primer
caggaaacagctatgactttgtgggatgggataagga

SEQ ID NO: 27 - DNA, 20 bp, Artificial Sequence
  Synthetic primer
ctccttgcttggaattctgc

SEQ ID NO: 28 - DNA, 20 bp, Artificial Sequence
  Synthetic primer
ttcagtccctcgttggtttc

SEQ ID NO: 29 - DNA, 43 bp, Artificial Sequence
  Synthetic primer
ctcgttgtaaaacgacggccagtagcttcgcggccgctctccc

SEQ ID NO: 30 - DNA, 49 bp, Artificial Sequence
  Synthetic primer
ctgctcaggaaacagctatgacctggcgctgagcaggaagaagatgagg

SEQ ID NO: 31 - DNA, 40 bp, Artificial Sequence
  Synthetic primer
ctcgttgtaaaacgacggccagtcctccgcggacggtctt

SEQ ID NO: 32 - DNA, 43 bp, Artificial Sequence
  Synthetic primer
ctgctcaggaaacagctatgaccgtctccatcgctagggtgtt

SEQ ID NO: 33 - DNA, 67 bp, Artificial Sequence
  Synthetic primer
gggttccctaagggttggactgccgggtgccggacgccgcgaacctgagcagcgcctggcgcaacca

SEQ ID NO: 34 - DNA, 71 bp, Artificial Sequence
  Synthetic primer
cactgtcccactgcggctgcgggacggccgcgaggtgccccacagctgtctagattggatcttgctggcac

SEQ ID NO: 35 - DNA, 50 bp, Artificial Sequence
  Synthetic primer
gggttccctaagggttggaatttcagggcagctgtcagacaggtaaggtg

SEQ ID NO: 36 - DNA, 54 bp, Artificial Sequence
  Synthetic primer
tctgtcttctggagcaccaggggacctcagctctagattggatcttgctggcac

SEQ ID NO: 37 - DNA, 48 bp, Artificial Sequence
  Synthetic primer
ctcgttgtaaaacgacggccagtaacacttactctctgcctgtctctc

SEQ ID NO: 38 - DNA, 48 bp, Artificial Sequence
  Synthetic primer
ctgctcaggaaacagctatgacctcacagatacattcagcttagacag

SEQ ID NO: 39 - DNA, 43 bp, Artificial Sequence
  Synthetic primer
ctcgttgtaaaacgacggccagttcttgagaaagccccacttg

SEQ ID NO: 40 - DNA, 43 bp, Artificial Sequence
  Synthetic primer
ctgctcaggaaacagctatgaccaggttcaccaggaagctctg

SEQ ID NO: 41 - DNA, 43 bp, Artificial Sequence
  Synthetic primer
ctcgttgtaaaacgacggccagtcagtcagacaggaggggttc

SEQ ID NO: 42 - DNA, 45 bp, Artificial Sequence
  Synthetic primer
ctgctcaggaaacagctatgaccaatctatgcttcctgtctctgg

SEQ ID NO: 43 - DNA, 43 bp, Artificial Sequence
  Synthetic primer
ctcgttgtaaaacgacggccagtcacaagctctggttctgcaa

SEQ ID NO: 44 - DNA, 43 bp, Artificial Sequence
  Synthetic primer
ctgctcaggaaacagctatgaccctgtgagcagggaggacttc

SEQ ID NO: 45 - DNA, 43 bp, Artificial Sequence
  Synthetic primer
ctcgttgtaaaacgacggccagtgctgagatgcagacagctaa

SEQ ID NO: 46 - DNA, 44 bp, Artificial Sequence
  Synthetic primer
ctgctcaggaaacagctatgaccttttgtctgaggcatgtctgt

SEQ ID NO: 47 - DNA, 44 bp, Artificial Sequence
  Synthetic primer
ctcgttgtaaaacgacggccagtgcagggttacagttactgctg

SEQ ID NO: 48 - DNA, 43 bp, Artificial Sequence
  Synthetic primer
ctgctcaggaaacagctatgacctaagcaagggagctgtgatg

SEQ ID NO: 49 - DNA, 46 bp, Artificial Sequence
  Synthetic primer
ctcgttgtaaaacgacggccagtgatagaaactgactccccaaaaa

SEQ ID NO: 50 - DNA, 43 bp, Artificial Sequence
  Synthetic primer
ctgctcaggaaacagctatgacccactgggacaagaacccttc

SEQ ID NO: 51 - DNA, 45 bp, Artificial Sequence
  Synthetic primer
ctcgttgtaaaacgacggccagttgtgagaccaagaaggaaagtg

SEQ ID NO: 52 - DNA, 43 bp, Artificial Sequence
  Synthetic primer
ctgctcaggaaacagctatgaccggctactgccatggagatgt

SEQ ID NO: 53 - DNA, 43 bp, Artificial Sequence
  Synthetic primer
ctcgttgtaaaacgacggccagtctgctgcaggattctcttcc

SEQ ID NO: 54 - DNA, 43 bp, Artificial Sequence
  Synthetic primer
ctgctcaggaaacagctatgacctgcttctcacattcctccag

SEQ ID NO: 55 - DNA, 18 bp, Artificial Sequence
  Synthetic primer
tgcacgtgtaccctagaa

SEQ ID NO: 56 - DNA, 18 bp, Artificial Sequence
  Synthetic primer
tgcacgtgtaccccagaa

SEQ ID NO: 57 - DNA, 18 bp, Artificial Sequence
  Synthetic primer
tgcacatgtaccccagaa

SEQ ID NO: 58 - DNA, 16 bp, Artificial Sequence
  Synthetic primer
tgcaacctcagtctac

SEQ ID NO: 59 - DNA, 16 bp, Artificial Sequence
  Synthetic primer
tgcaacctcaatctat

SEQ ID NO: 60 - DNA, 16 bp, Artificial Sequence
  Synthetic primer
tccaacctcaatctat

SEQ ID NO: 61 - DNA, 14 bp, Artificial Sequence
  Synthetic primer
taaacatatgtgtg

SEQ ID NO: 62 - DNA, 14 bp, Artificial Sequence
  Synthetic primer
taaacaaacatggt

SEQ ID NO: 63 - DNA, 14 bp, Artificial Sequence
  Synthetic primer
ctgactaacatggt

SEQ ID NO: 64 - DNA, 59 bp, Artificial Sequence
  Synthetic primer
gggttccctaagggttggagtcactctccttttcttcccaggtttggccggaagaatgt

SEQ ID NO: 65 - DNA, 63 bp, Artificial Sequence
  Synthetic primer
gctgttcgtgaccatgggcatgcagacaggcttcagcttctctagattggatcttgctggcac

SEQ ID NO: 66 - DNA, 65 bp, Artificial Sequence
  Synthetic primer
gggttccctaagggttggactgctaactcgacctcccttgttttgaacagggacagaaattcttg

SEQ ID NO: 67 - DNA, 69 bp, Artificial Sequence
  Synthetic primer
gcaagtcagttcgtataatattctctacgttaggagtgtgcatatttctagattggatcttgctggcac

SEQ ID NO: 68 - DNA, 55 bp, Artificial Sequence
  Synthetic primer
gggttccctaagggttggatgggtgagagggacagactgaccgtgatttgagagc

SEQ ID NO: 69 - DNA, 59 bp, Artificial Sequence
  Synthetic primer
agcagcacccagccctgaagtcctccctgctcacagtctagattggatcttgctggcac

SEQ ID NO: 70 - DNA, 48 bp, Artificial Sequence
  Synthetic primer
gggttccctaagggttggacagtcccacaacattctggatctgcttcg

SEQ ID NO: 71 - DNA, 52 bp, Artificial Sequence
  Synthetic primer
aacctggaatatccggatggtcaccatcatctagattggatcttgctggcac

SEQ ID NO: 72 - DNA, 69 bp, Artificial Sequence
  Synthetic primer
gggttccctaagggttggaccccggcgctattccatggccactgccctcttcctgggtggcagtgtcct

SEQ ID NO: 73 - DNA, 73 bp, Artificial Sequence
  Synthetic primer
tctcttcatgcagctggtacccccaggtagggaccatgtgcatctatggttctagattggatcttgctggcac

SEQ ID NO: 74 - DNA, 74 bp, Artificial Sequence
  Synthetic primer
gggttccctaagggttggatgcaggtcgaaaaatgggtggatggcgaaagtgcatgtccttatagctgcaggtc

SEQ ID NO: 75 - DNA, 78 bp, Artificial Sequence
  Synthetic primer
ccagcctcctttcagcaatcaatttggagaggaaagcaatactctgggacacgtatctagattggatcttgctggcac

SEQ ID NO: 76 - DNA, 57 bp, Artificial Sequence
  Synthetic primer
gggttccctaagggttggataagaagacctcctctgtcagtgttgatgcactgggtc

SEQ ID NO: 77 - DNA, 61 bp, Artificial Sequence
  Synthetic primer
tgggtctggccaggtctcaggagcccctcacaatagagtctagattggatcttgctggcac

SEQ ID NO: 78 - DNA, 76 bp, Artificial Sequence
  Synthetic primer
gggttccctaagggttggatattacgcccgtgccttatccggagagggcagatcaccagcaaagtgcactgatgtg

SEQ ID NO: 79 - DNA, 80 bp, Artificial Sequence
  Synthetic primer
tgagctcttaagaccactcagcatgacgaatgaatgacgcgacaggaagaacttgactctagattggatcttgctggcac

SEQ ID NO: 80 - DNA, 20 bp, Artificial Sequence
  Synthetic primer
tacactcccaccaacactgt

SEQ ID NO: 81 - DNA, 19 bp, Artificial Sequence
  Synthetic primer
tacactcccaccaacagtg

SEQ ID NO: 82 - DNA, 20 bp, Artificial Sequence
  Synthetic primer
tacattcccaccaacagtgt

SEQ ID NO: 83 - DNA, 18 bp, Artificial Sequence
  Synthetic primer
gttcttgtcctagggtgt

SEQ ID NO: 84 - DNA, 18 bp, Artificial Sequence
  Synthetic primer
gttcttgtcctactaaaa

SEQ ID NO: 85 - DNA, 18 bp, Artificial Sequence
  Synthetic primer
accctgtctctactaaaa